(19) United States
(12) Patent Application Publication
(10) Pub. No.: US 2024/0317939 A1
(43) Pub. Date: Sep. 26, 2024

(54) METHODS FOR PRODUCING OF LIPIDS

(71) Applicant: Pfizer Inc., New York, NY (US)

(72) Inventors: Adam Ross Brown, Old Lyme, CT (US); Shane Allen Eisenbeis, Pawcatuck, CT (US); Aran Kathleen Hubbell, Groton, CT (US); Ruizhi Li, Colchester, CT (US); Giselle Padilla Reyes, Pawcatuck, CT (US); Philipp Christopher Roosen, Niantic, CT (US)

(21) Appl. No.: 18/261,128

(22) PCT Filed: Jan. 12, 2022
(86) PCT No.: PCT/IB2022/050207

§ 371 Date: Jul. 12, 2023

Related U.S. Application Data
(60) Provisional application No. 63/178,411, filed on Apr. 22, 2021.
(60) Provisional application No. 63/138,086, filed on Jan. 15, 2021.

(51) Int. Cl. C08G 69/40; A61K 9/127; A61K 47/54

(52) U.S. Cl. CPC C08G 69/40; A61K 9/127; A61K 47/543

(57) ABSTRACT

Methods for producing a compound of Formula I, Formula I, wherein R¹ and R² are independently a i) linear or branched or cyclic, ii) saturated or unsaturated, and iii) substituted or unsubstituted hydrocarbon group comprising 8 to 20 carbon atoms; R³ is a hydrocarbon group; n is an integer from 2 to 5, m is an integer from 30 to 70, and L is a linker. The method includes: a) contacting a fatty acid having a chemical formula of R¹—COOH and a primary amine having a chemical formula of R²—NH₂ to form an amide having a chemical formula of R¹—C(O)—NH—R²; b) contacting the amide with a reducing agent to form a secondary amine having a chemical formula of R¹—CH₂—NH—R²; and c) contacting the secondary amine with a polyolefin-glycol compound to form the compound of Formula I. Intermediates produced in the method, salts of compound of Formula I and of intermediates.

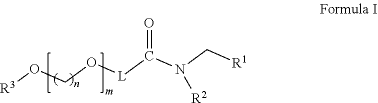

### FIELD OF THE INVENTION

The disclosure relates to methods for producing lipid compounds or intermediates thereof or pharmaceutically acceptable salts thereof. Some aspects relate to salts of the lipid compounds or intermediates thereof. The lipids and/or pharmaceutically acceptable salts thereof in combination with other lipids can be used for intracellular delivery of nucleic acids.

### BACKGROUND

Nucleic acid based therapeutics have enormous potential. Although free or naked nucleic acids can be used in some instances to transfect cells (Wolff et al. 1990, Science, 247, 1465-1468), it is generally advantageous or necessary to formulate the nucleic acid with at least a second agent that protects the nucleic acid from degradation during delivery, facilitates distribution to and in a target tissue, facilitates cellular uptake, and/or enables suitable intracellular processing. Free RNAs can be unstable, susceptible to nuclease digestion, and can have limited ability to gain access to the target tissue, cells, and/or intracellular compartments where the relevant translation machinery resides.

Lipids such as polymer conjugated lipids have been used for intracellular delivery of nucleic acids. Lipid containing nanoparticles containing encapsulated nucleic acids, are generally well-tolerated and can be used for targeted delivery of nucleic acids in a patient. For Example, U.S. Pat. No. 10,166,298 describes various lipids, that can used for targeted delivery of various nucleic acids, such as messenger RNA (mRNA), antisense oligonucleotides, ribozymes, DNAzymes, plasmids, immune stimulating nucleic acids, antagomirs, anti-miRs, miRNA mimics, supermirs, and aptamers.

However, current methods for producing such lipids can be time consuming. Thus, there remains a need for relatively fast and cost effective preparation methods of lipids with high purity, such as lipids that can be used for nucleic acid delivery.

### SUMMARY

Applicant discloses solutions to at least some of the aforementioned problems associated with producing polymer conjugated lipids and intermediates for the production thereof. In one aspect, Applicant discloses producing polymer conjugated lipids and/or intermediates for the production thereof in a shorter amount of time than previously achieved. In some instances, the amount of time needed to produce the final product and/or intermediates for the production thereof is shortened in comparison due to one or more reaction steps using different reagents and/or reaction conditions than those used previously to produce a polymer conjugated lipid. In some instances, the amount of time needed to produce the final product and/or intermediates for the production thereof is shortened in comparison due to formation of crystals and/or solid precipitates of the intermediates and/or final product. In another aspect, Applicant discloses producing polymer conjugated lipids with high purity where the method does not involve solvent lyophilization to isolate the final product and/or intermediates in solid form. In another aspect, Applicant discloses salts of the polymer conjugated lipids and intermediates for the production thereof. In some instances, the salts can be pharmaceutically acceptable, be environmentally safe, and/or have improved solubility or insolubility, bioavailability, purity, and/or steps for removal and/or replacement of the salt.

Those skilled in the art will recognize, or be able to ascertain using no more than routine experimentation, many equivalents to the specific aspects described herein. Such equivalents are intended to be encompassed by the following aspects.
Aspect 1 is directed to a method for producing a compound having a chemical formula of Formula I

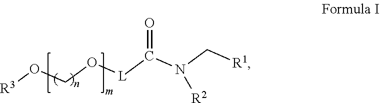

wherein R¹ and R² are independently a i) linear or branched or cyclic, ii) saturated or unsaturated, and iii) substituted or unsubstituted hydrocarbon group comprising 8 to
20 carbon atoms,
R³ is a hydrocarbon group,
n is an integer from 2 to 5,
m is an integer from 30 to 70, and
L is a linker,
the method comprising:
   a) forming an amide having a chemical formula of R¹—C(O)—NH—R² from a fatty acid having a chemical formula of R¹—COOH and a primary amine having a chemical formula of R²—NH₂;
   b) contacting the amide with a reducing agent to form a secondary amine having a chemical formula of R¹—CH₂—NH—R²; and
   c) contacting the secondary amine with a polyolefin-glycol compound to form the compound of Formula I.
Aspect 2 is directed to the method of aspect 1, wherein the fatty acid is contacted with 1,1′-Carbonyldiimidazole (CDI) to form a N-acyl imidazole having the chemical formula of of R¹—C(O)—C₃N₂H₄, and the N-acyl imidazole is contacted with the primary amine to form the amide.
Aspect 3 is directed to the method of aspect 2, wherein the fatty acid and CDI have a molar ratio of 1:1.2 to 1.2:1.
Aspect 4 is directed to the method of any one of aspects 2 to 3, wherein contacting the fatty acid with CDI is performed at a temperature of 40° C. to 60° C.
Aspect 5 is directed to the method of any one of aspects 2 to 4, wherein the fatty acid and CDI are contacted in the presence of toluene.
Aspect 6 is directed to the method of any one of aspects 1 to 5, wherein the N-acyl imidazole and the primary amine have a molar ratio of 0.9:1 to 1:0.9.
Aspect 7 is directed to the method of any one of aspects 2 to 6, wherein the amount of primary amine contacted with the N-acyl imidazole is 0.85 to 1.2 moles of primary amine per mole of the fatty acid used to form the N-acyl imidazole.
Aspect 8 is directed to the method of any one of aspects 1 to 7, wherein at least a portion of the primary amine is in a melted form.
Aspect 9 is directed to the method of any one of aspects 1 to 8, wherein the N-acyl imidazole and the primary amine are contacted at a temperature of 40° C. to 60° C.
Aspect 10 is directed to the method of aspect 1, wherein the fatty acid is contacted with an oxychloride to form an acyl chloride having a chemical formula of R¹—C(O)—Cl, and the acyl chloride is contacted with the primary amine to form the amide, wherein the oxychloride is selected from thionyl chloride, phosphoryl chloride, oxalyl chloride, and any combinations thereof.
Aspect 11 is directed to the method of aspect 10, wherein the fatty acid and the oxychloride have a molar ratio of 1:0.8 to 1:2.
Aspect 12 is directed to the method of any one of aspects 10 to 11, wherein the fatty acid and the oxychloride are contacted in the presence of benzene and dimethylformamide.
Aspect 13 is directed to the method of any one of aspects 10 to 12, wherein the fatty acid and the oxychloride are contacted at a temperature of 20° C. to 75° C.
Aspect 14 is directed to the method of any one of aspects 10 to 13, wherein the oxychloride is oxalyl chloride.
Aspect 15 is directed to the method of any one of aspects 10 to 14, wherein the acyl chloride and primary amine are contacted at a temperature of 2° C. to 20° C.
Aspect 16 is directed to the method of any one of aspects 10 to 15, wherein the acyl chloride and primary amine are contacted in the presence of benzene and triethylamine.
Aspect 17 is directed to the method of any one of aspects 10 to 16, wherein the amount of primary amine contacted with the acyl chloride is 0.6 to 1.2 moles of primary amine per mole of the fatty acid used to form the acyl chloride.
Aspect 18 is directed to the method of any one of aspects 1 to 17, further comprising crystallizing the amide from an amidation-product mixture formed in step (a), and using the crystallized amide as at least a portion of the amide in step (b).
Aspect 19 is directed to the method of aspect 18, wherein crystallizing the amide comprises adding isopropanol to the amidation-product mixture to form a crystallization mixture and cooling the crystallization mixture.
Aspect 20 is directed to the method of aspect 19, wherein crystallizing the amide comprises:
   contacting isopropanol with the amidation-product mixture at a temperature greater than 40° C. and at or below 60° C. to form the crystallization mixture;
   cooling the crystallization mixture to a temperature of 30° C. to 40° C. to form a slurry containing amide crystals;
   maintaining the slurry at a temperature of 30° C. to 40° C., with continuous, periodic, or occasional stirring for at least 1 hour;
   cooling the slurry to a temperature of 15° C. to 25° C. with continuous, periodic, or occasional stirring;
   maintaining the slurry at a temperature of 15° C. to 25° C. with continuous, periodic, or occasional stirring for at least 0.5 hour; and separating the amide crystals from the slurry.
Aspect 21 is directed to the method of aspect 20, wherein the slurry is cooled to a temperature of 15° C. to 25° C. with continuous stirring at 600 rpm or above.
Aspect 22 is directed to the method of any one of aspects 18 to 21, wherein the amide crystals are separated from the slurry by filtration.
Aspect 23 is directed to the method of aspect 22, further comprising washing the filtered amide crystals with toluene and/or isopropanol, and drying the washed crystals.
Aspect 24 is directed to the method of aspect 23, wherein the amide crystals are 15 dried at a temperature of 40° C. to 50° C.
Aspect 25 is directed to the method of any one of aspects 18 to 24, wherein the amide crystallization process is performed in a reactor having a diameter D, and the reactor comprises an impeller having a diameter D₁, and D₁:D is 0.35:1 to 0.65:1.
Aspect 26 is directed to the method of aspect 25, wherein the slurry in the reactor has a height H, and H is less than D.
Aspect 27 is directed to the method of any one of aspects 18 to 26, wherein the amidation-product mixture comprises less than 4% by weight of the starting primary amine.
Aspect 28 is directed to the method of any one of aspects 1 to 27, wherein the reducing agent is a hydride.
Aspect 29 is directed to the method of aspect 28, wherein the hydride is lithium aluminum hydride.
Aspect 30 is directed to the method of any one of aspects 1 to 29, wherein the amide and the reducing agent have a molar ratio of 1:1 to 1:3.
Aspect 31 is directed to the method of any one of aspects 1 to 30, wherein in step (b) an amide solution comprising the amide is contacted with a reducing agent solution comprising the reducing agent.
Aspect 32 is directed to the method of aspect 31, wherein the amide solution further comprises toluene and/or the reducing agent solution further comprises 2-methyl tetrahydrofuran (THF) and/or THF.
Aspect 33 is directed to the method of any one of aspects 31 to 32, wherein the amide solution is formed by contacting crystals of the amide with toluene.
Aspect 34 is directed to the method of anyone of aspects 1 to 33, wherein contacting of the amide and the reducing agent is performed at a temperature of 50° C. to 75° C.
Aspect 35 is directed to the method of any one of aspects 1 to 34, wherein step (b) the amide is reduced to form the secondary amine, and the step (b) further comprises quenching the reduction of the amide by adding sodium sulfate.
Aspect 36 is directed to the method of aspect 35, wherein quenching the reduction of the amide comprises:
   contacting a reduction-product mixture formed in step (b) with a slurry comprising sodium sulfate at a temperature of 35° C. to 45° C. to form a quenched reduction-product mixture and residual sodium sulfate;
   separating at least a portion of the residual sodium sulfate from the quenched reduction-product mixture to form a separated reduction-product mixture comprising the secondary amine.
Aspect 37 is directed to the method of aspect 36, wherein the reduction-product mixture comprises less than 4% by weight of the starting amide.
Aspect 38 is directed to the method of any one of aspects 35 to 37, wherein 0.5 to 2 moles of sodium sulfate per mole of amide is added.
Aspect 39 is directed to the method of any one of aspects 36 to 38, wherein the slurry comprising sodium sulfate further comprises THF and/or toluene.
Aspect 40 is directed to the method of any one of aspects 36 to 39, wherein the at least a portion of the residual sodium sulfate is separated from the quenched reduction-product mixture by filtration, wherein the separated reduction-product mixture is formed as a filtrate.
Aspect 41 is directed to the method of any one of aspects 1 to 40, further comprising forming a crystallized salt of the secondary amine.
Aspect 42 is directed to the method of aspect 41, wherein the crystallized salt of the secondary amine is formed by a process comprising, contacting the secondary amine with an acid to form a salt-forming solution comprising a salt of the secondary amine, and cooling the salt-forming solution to form the crystallized salt of the secondary amine.
Aspect 43 is directed to the method of any one of aspects 41 or 42, wherein the crystallized salt of the secondary amine is formed by a process comprising:
   contacting the secondary amine with isopropanol and an acid at a temperature of 50° C. to 60° C. to form a salt-forming solution comprising a salt of the secondary amine;
   cooling the salt-forming solution to 30° C. to 45° C. to form salt crystals;
   maintaining the salt-forming solution at 30° C. to 45° C. for at least 1 hour;
   cooling the salt-forming solution to 15° C. to 25° C.;
   separating the salt crystals from the salt-forming solution.
Aspect 44 is directed to the method of aspect 43, wherein the salt crystals are separated from the salt-forming solution by filtering, wherein the crystallized salt is obtained as filtered residue.
Aspect 45 is directed to the method of aspect 44, further comprising washing and drying the filtered residue to form a dried, crystallized salt of the secondary amine.
Aspect 46 is directed to the method of aspect 45, wherein the filtered residue is washed with a toluene and/or isopropanol solution.
Aspect 47 is directed to the method of aspect 46, wherein volume % ratio of the toluene and isopropanol in the toluene and/or isopropanol solution is 0.9:1 to 1:0.9.
Aspect 48 is directed to the method of any one of aspects 45 to 47, wherein the filtered residue is dried at a pressure of 0 to 0.2 bar and/or a temperature of 40° C. to 50° C.
Aspect 49 is directed to the method of any one of aspects 42 to 48, wherein the acid is hydrochloric acid, hydrobromic acid, hydroiodic acid, sulfuric acid, acetic acid, methanesulfonic acid, toluenesulfonic acid, (1R)-(-)-10-camphorsulfonic acid, 1,2-ethanedisulfonic acid, oxalic acid, dibenzoyl-L-tartaric acid, phosphoric acid, L-tartaric acid, maleate, fumaric acid, succinic acid, and/or malonic acid.
Aspect 50 is directed to the method of any one of aspects 42 to 49, wherein the acid is succinic acid.
Aspect 51 is directed to the method of any one of aspects 45 to 50, wherein the secondary amine is reformed from the dried, crystallized salt of the secondary amine, and the reformed secondary amine is used in step (c).
Aspect 52 is directed to the method of aspect 51, wherein the secondary amine is reformed from the dried, crystallized salt by contacting the dried, crystallized salts with a base.
Aspect 53 is directed to the method of aspect 51, wherein the secondary amine is reformed from the dried, crystallized salt of the secondary amine by a process comprising:
   contacting the dried, crystallized salt of the secondary amine with an organic solvent to form a salt solution,
   washing the salt solution with a base and water to form a washed organic solution comprising the secondary amine; and
   distilling the washed organic solution to form a distilled organic solution.
Aspect 54 is directed to the method of aspect 53, wherein the salt solution is washed with the base and/or water more than once.
Aspect 55 is directed to the method of any one of aspects 53 to 54, wherein the dried, crystallized salt is contacted with toluene to form the salt solution.
Aspect 56 is directed to the method of any one of aspects 53 to 55, wherein the washed organic solution comprises less than 100 μg per mL of the acid.
Aspect 57 is directed to the method of any one of aspects 53 to 56, wherein the washed organic solution is distilled at a pressure of 0 to 0.3 bar and/or a temperature at or below 70° C. to form the distilled organic solution.
Aspect 58 is directed to the method of any one of aspects 53 to 57, wherein the distilled organic solution comprises less than 0.05 wt. % of water.
Aspect 59 is directed to the method of any one of aspects 53 to 58, wherein the base is NaOH and/or KOH.
Aspect 60 is directed to the method of any one of aspects 53 to 59, wherein the reformed secondary amine in the distilled organic phase is used in step (c).
Aspect 61 is directed to the method of any one of aspects 1 to 60, wherein the polyolefin-glycol compound is a polyolefin glycol acid having a chemical formula of HOOC—L—(O(CH₂)ₙ)ₘ—OR³.
Aspect 62 is directed to the method of aspect 61, wherein the secondary amine and the polyolefin glycol acid have a molar ratio of 1:1.2 to 1:1.5.
Aspect 63 is directed to the method of any one of aspects 61 to 62, wherein the polyolefin glycol acid is activated by contacting the polyolefin glycol acid with an organic base and a coupling agent to form a coupling solution comprising an activated polyolefin-glycol compound, and the coupling solution is contacted with the secondary amine.
Aspect 64 is directed to the method of aspect 63, wherein the organic base is a tertiary amine.
Aspect 65 is directed to the method of aspect 64, wherein the tertiary amine is diisopropylethylamine.
Aspect 66 is directed to the method of any one of aspects 63 to 65, wherein the coupling agent is 1-propanephosphonic acid cyclic anhydride.
Aspect 67 is directed to the method of any one of aspects 63 to 66, wherein the coupling solution is colorless.
Aspect 68 is directed to the method of any one of aspects 63 to 67, wherein the coupling solution is formed by contacting the polyolefin glycol acid and the organic base at a molar ratio of 1:3.5 to 1:4.5.
Aspect 69 is directed to the method of any one of aspects 63 to 68, wherein the coupling solution is formed by contacting the polyolefin glycol acid and the coupling agent at a molar ratio of 1:1.8 to 1:2.2.
Aspect 70 is directed to the method of any one of aspects 63 to 69, wherein the coupling solution is formed by contacting the polyolefin glycol acid with an organic solvent to form a polyolefin glycol solution, distilling the polyolefin glycol solution to form a distilled polyolefin glycol solution, and contacting the distilled polyolefin glycol solution with the base and coupling agent to form the coupling solution.
Aspect 71 is directed to the method of aspect 70, wherein the polyolefin glycol solution is distilled at a pressure of 0 to 0.2 bar and/or a temperature at or below 70° C.
Aspect 72 is directed to the method of any one of aspects 70 to 71, wherein the distilled polyolefin glycol solution contains less than 0.05 wt. % of water.
Aspect 73 is directed to the method of any one of aspects 70 to 72, wherein the polyolefin glycol acid is contacted with toluene to form the polyolefin glycol solution.
Aspect 74 is directed to the method of any one of aspects 63 to 73, wherein in step (c) the coupling solution is contacted with a reformed secondary amine.
Aspect 75 is directed to the method of any one of aspects 63 to 74, wherein in step (c) the coupling solution is contacted with a reformed secondary amine from the distilled organic solution.
Aspect 76 is directed to the method of any one of aspects 1 to 75, wherein in step 15 (c) the compound of Formula I is formed at a temperature of 20° C. to 45° C.
Aspect 77 is directed to the method of any one of aspects 1 to 60, wherein the polyolefin-glycol compound is a N-hydroxylsuccinimide (NHS) functionalized polyolefin glycol, and has a chemical formula of NHS—O(O)C—L—(O(CH₂)ₙ)ₘ—OR³.
Aspect 78 is directed to the method of aspect 77, wherein the NHS functionalized polyolefin glycol is contacted with the secondary amine at a molar ratio 0.6:1 to 1.2:1.
Aspect 79 is directed to the method of any one of aspects 77 to 78, wherein the NHS functionalized polyolefin glycol is contacted with the secondary amine in presence of a tertiary amine.
Aspect 80 is directed to the method of aspect 79, wherein the tertiary amine is triethylamine.
Aspect 81 is directed to the method of any one of aspects 77 to 80, wherein the NHS functionalized polyolefin glycol is contacted with the secondary amine at a temperature of 20° C. to 45° C.
Aspect 82 is directed to the method of any one of aspects 77 to 81, wherein the NHS functionalized polyolefin glycol is contacted with a reformed secondary amine.
Aspect 83 is directed to the method of any one of aspects 77 to 82, wherein the NHS functionalized polyolefin glycol is contacted with a reformed secondary amine from a distilled organic solution.
Aspect 84 is directed to the method of any one of aspects 1 to 83, wherein step (c) comprises coupling of the polyolefin-glycol compound and the secondary amine, and step (c) further comprises quenching the coupling by adding an aqueous quench solution comprising potassium carbonate and sodium chloride.
Aspect 85 is directed to the method of aspect 84, wherein quenching the coupling comprises:
   contacting the aqueous quench solution with a coupling-product mixture formed in step (c) to form a biphasic product mixture comprising i) an organic phase comprising the compound of Formula I and less than 10 wt. % of the secondary amine, and ii) an aqueous phase,
   separating the organic phase and the aqueous phase of the biphasic product mixture, and
   distilling the organic phase to form a product solution comprising the compound of Formula I and less than 0.12 wt. % of water.
Aspect 86 is directed to the method of embodiment 85, wherein the organic phase is distilled at a pressure of 0 to 0.2 bar and/or a temperature at or below 70° C. to form the product solution.
Aspect 87 is directed to the method of any one of aspects 1 to 86, wherein the method further comprises at least partially purifying the compound of Formula I.
Aspect 88 is directed to the method of aspect 87, wherein the compound of Formula I is at least partially purified by silica gel chromatography or polymer resin chromatography.
Aspect 89 is directed to the method of any one of aspects 87 to 88, further comprising precipitating the compound of Formula I, the process comprising:
   obtaining an ethanol solution of the at least partially purified compound of Formula I,
   contacting isopropanol with the ethanol solution to form an isopropanol and ethanol mixture, wherein the compound of Formula I precipitates from the isopropanol and ethanol mixture, and
   separating the precipitate of the compound of Formula I from the isopropanol and ethanol mixture.
Aspect 90 is directed to the method of aspect 89, wherein isopropanol is contacted with the ethanol solution at an isopropanol:ethanol weight ratio of 3.5:1 to 2.5:1.
Aspect 91 is directed to the method of any one of aspects 89 to 90, wherein the precipitate of the compound of Formula I is separated from the isopropanol and ethanol mixture by filtration.
Aspect 92 is directed to the method of any one of aspects 1 to 91, wherein the n is 2 and m is 40 to 50.
Aspect 93 is directed to the method of any one of aspects 1 to 92, wherein R³ is an alkyl group.
Aspect 94 is directed to the method of any one of aspects 1 to 93, wherein R³ is a methyl group.
Aspect 95 is directed to the method of any one of aspects 1 to 94, wherein R¹ and R² are independently a linear, saturated, and unsubstituted alkyl group.
Aspect 96 is directed to the method of any one of aspects 1 to 95, wherein R¹ and R² independently have a chemical formula selected from the group —(CH₂)₇CH₃, —(CH₂)₈CH₃, —(CH₂)₉CH₃, —(CH₂)₁₀CH₃, —(CH₂)₁₁CH₃, —(CH₂)₁₂CH₃, —(CH₂)₁₃CH₃, —(CH₂)₁₄CH₃, —(CH₂)₁₅CH₃, —(CH₂)₁₆CH₃, —(CH₂)₁₇CH₃, —(CH₂)₁₈CH₃, and —(CH₂)₁₉CH₃.
Aspect 97 is directed to the method of any one of aspects 1 to 96, wherein R¹ is —(CH₂)₁₂CH₃ and/or R² is —(CH₂)₁₃CH₃ group.
Aspect 98 is directed to the method of any one of aspects 1 to 97, wherein L has a chemical formula of —(CH₂)^{a′}—X— (CH₂)_{a″}—, wherein a′ and a″ are independently 0, 1, 2, 3, 4, or 5, and X is a linker.
Aspect 99 is directed to the method of aspect 98, wherein X is a bond, —HC═CH—, —C≡C—, —C₆H₄—, —O—, or —S—.
Aspect 100 is directed to the method of any one of aspects 1 to 99, wherein L is —CH₂-.
Aspect 101 is directed to the method of any one of aspects 1 to 100, wherein Formula I is Formula Ia

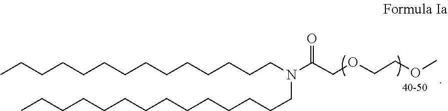

Aspect 102 is directed to a method for forming an amide having a chemical formula of R¹—C(O)—NH—R², wherein R¹ and R² are independently a i) linear or branched or cyclic, ii) saturated or unsaturated, and iii) substituted or unsubstituted hydrocarbon group comprising 8 to 20 carbon atoms, the method comprising
   a) contacting a fatty acid having a chemical formula of R¹—COOH with 1,1′-Carbonyldiimidazole (CDI) to form a N-acyl imidazole having a chemical formula of R¹—C(O)—C₃N₂H₄; and
   b) contacting the N-acyl imidazole with a primary amine having a chemical formula of R²—NH₂ to form an amide having a chemical formula of R¹—C(O)—NH—R².
Aspect 103 is directed to the method of aspect 102, wherein the fatty acid and CDI have a molar ratio of 1:1.2 to 1.2:1.
Aspect 104 is directed to the method of any one of aspects 102 to 103, wherein the 20 N-acyl imidazole is formed at a temperature of 40° C. to 60° C.
Aspect 105 is directed to the method of any one of aspects 102 to 104, wherein the fatty acid and CDI are contacted in presence of toluene.
Aspect 106 is directed to the method of any one of aspects 102 to 105, wherein the N-acyl imidazole and the primary amine have a molar ratio of 0.9:1 to 1:0.9.
Aspect 107 is directed to the method of any one of aspects 102 to 106, wherein the amount of primary amine contacted with the N-acyl imidazole is 0.85 to 1.2 moles of primary amine per mole of the fatty acid used to form the N-acyl imidazole.
Aspect 108 is directed to the method of any one of aspects 102 to 107, wherein at least a portion of the primary amine is in a melted form.
Aspect 109 is directed to the method of any one of aspects 102 to 108, wherein the amide is formed at a temperature of 40° C. to 60° C.
Aspect 110 is directed to the method of any one of aspects 102 to 109, further comprising crystallizing the amide from an amidation-product mixture formed by the reaction of the N-acyl imidazole and primary amine.
Aspect 111 is directed to the method of aspect 110, wherein crystallizing the amide comprises adding isopropanol to the amidation-product mixture to form a crystallization mixture and cooling the crystallization mixture.
Aspect 112 is directed to the method of aspect 110, wherein crystallizing the amide comprises:
   contacting isopropanol with the amidation-product mixture at a temperature greater than 40° C. and at or below 60° C. to form a crystallization mixture;
   cooling the crystallization mixture to a temperature of 30° C. to 40° C. to form a slurry containing amide crystals;
   maintaining the slurry at a temperature of 30° C. to 40° C., with continuous, periodic, or occasional stirring for at least 1 hour;
   cooling the slurry to a temperature of 15° C. to 25° C. with continuous, periodic, or occasional stirring;
   maintaining the slurry at a temperature of 15° C. to 25° C. with continuous, periodic, or occasional stirring for at least 0.5 hour; and separating the amide crystals from the slurry.
Aspect 113 is directed to the method of aspect 112, wherein the slurry is cooled to a temperature of 15° C. to 25° C. with continuous stirring at 600 rpm or above.
Aspect 114 is directed to the method of any one of aspects 112 to 113, wherein the amide crystals are separated from the slurry by filtration.
Aspect 115 is directed to the method of aspect 114, further comprising washing the filtered amide crystals with toluene and/or isopropanol, and drying the washed crystals.
Aspect 116 is directed to the method of aspect 115, wherein the amide crystals are dried at a temperature of 40° C. to 50° C.
Aspect 117 is directed to the method of any one of aspects 110 to 116, wherein the crystallization process is performed in a reactor having a diameter D, the slurry in the reactor has a height H, and H is less than D.
Aspect 118 is directed to the method of aspect 117, wherein the reactor comprises an impeller having a diameter D₁, and D₁:D is 0.35:1 to 0.65:1.
Aspect 119 is directed to the method of any one of aspects 110 to 118, wherein the amidation-product mixture comprises less than 4% by weight of the starting primary amine.
Aspect 120 is directed to the method of any one of aspects 102 to 119, wherein R¹ and R² are independently a linear, saturated, and unsubstituted alkyl group.
Aspect 121 is directed to the method of any one of aspects 102 to 120, wherein R¹ is —(CH₂)₁₂CH₃, and/or R² is —(CH₂)₁₃CH₃ group.
Aspect 123 is directed to a method for forming an amide having a chemical formula 15 of R¹—C(O)—NH—R², wherein R¹ and R² are independently a i) linear or branched or cyclic, ii) saturated or unsaturated, and iii) substituted or unsubstituted hydrocarbon group comprising 8 to 20 carbon atoms, the method comprising:
   a) contacting a fatty acid having a chemical formula of R¹—COOH with an oxychloride to form an acyl chloride having a chemical formula of R¹—C(O)—Cl, wherein the oxychloride is selected from the group thionyl chloride, phosphoryl chloride, oxalyl chloride, and any combinations thereof, and
   c) contacting the acyl chloride with a primary amine having a chemical formula of R²—NH₂ to form an amide having a chemical formula of R¹—C(O)—NH—R².
Aspect 123 is directed to the method of aspect 122, wherein the fatty acid and the oxychloride have a molar ratio of 1:0.8 to 1:2.
Aspect 124 is directed to the method of any one of aspects 122 to 123, wherein the fatty acid and the oxychloride are contacted in presence of benzene and dimethylformamide.
Aspect 125 is directed to the method of any one of aspects 122 to 124, wherein the fatty acid and the oxychloride are contacted at a temperature of 20° C. to 75° C.
Aspect 126 is directed to the method of any one of aspects 122 to 125, wherein the oxychloride is oxayl chloride.
Aspect 127 is directed to the method of any one of aspects 122 to 126, wherein the acyl chloride and primary amine are contacted at a temperature of 2° C. to 20° C.
Aspect 128 is directed to the method of any one of aspects 122 to 127, wherein the acyl chloride and primary amine are contacted in presence of benzene and triethylamine.
Aspect 129 is directed to the method of any one of aspects 122 to 128, wherein the amount of primary amine contacted with the acyl chloride is 0.6 to 1.2 moles of primary amine per mole of the fatty acid used to form the acyl chloride.
Aspect 130 is directed to the method of any one of aspects 122 to 129, further comprising crystallizing the amide from an amidation-product mixture formed by the reaction of the acyl chloride and primary amine.
Aspect 131 is directed to the method of aspect 130, wherein crystallizing the amide comprises adding isopropanol to the amidation-product mixture to form a crystallization mixture and cooling the crystallization mixture.
Aspect 132 is directed to the method of aspect 130, wherein crystallizing the amide comprises:
   contacting isopropanol with the amidation-product mixture at a temperature greater than 40° C. and at or below 60° C. to form a crystallization mixture;
   cooling the crystallization mixture to a temperature of 30° C. to 40° C. to form a slurry containing amide crystals;
   maintaining the slurry at a temperature of 30° C. to 40° C., with continuous, periodic, or occasional stirring for at least 1 hour;
   cooling the slurry to a temperature of 15° C. to 25° C. with continuous, periodic, or occasional stirring;
   maintaining the slurry at a temperature of 15° C. to 25° C. with continuous, periodic, or occasional stirring for at least 0.5 hour; and separating the amide crystals from the slurry.
Aspect 133 is directed to the method of aspect 132, wherein the slurry is cooled to a temperature of 15° C. to 25° C. with continuous stirring at 600 rpm or above.
Aspect 134 is directed to the method of any one of aspects 132 to 133, wherein the amide crystals are separated from the slurry by filtration.
Aspect 135 is directed to the method of aspect 134, further comprising washing the filtered amide crystals with toluene and/or isopropanol, and drying the washed crystals.
Aspect 136 is directed to the method of aspect 135, wherein the amide crystals are dried at a temperature of 40° C. to 50° C.
Aspect 137 is directed to the method of any one of aspects 130 to 136, wherein the crystallization process is performed in a reactor having a diameter D, the slurry in the reactor having a height H, and H is less than D.
Aspect 138 is directed to the method of aspect 137, wherein the reactor comprises an impeller having a diameter D₁, and D₁:D is 0.35:1 to 0.65:1.
Aspect 139 is directed to the method of any one of aspects 130 to 138, wherein the amidation-product mixture comprises less than 4% by weight of the starting primary amine.
Aspect 140 is directed to the method of any one of aspects 122 to 139, wherein R¹ and R² are independently a linear, saturated, and unsubstituted alkyl group.
Aspect 141 is directed to the method of any one of aspects 122 to 140, wherein R¹ is —(CH₂)₁₂CH₃, and/or R² is —(CH₂)₁₃CH₃ group.
Aspect 142 is directed to a method for producing a compound having a chemical formula of Formula I in solid phase

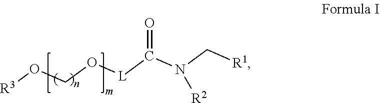

wherein R¹ and R² are independently a i) linear or branched or cyclic, ii) saturated or unsaturated, and iii) substituted or unsubstituted hydrocarbon group comprising 8 to 20 carbon atoms,
R³ is a hydrocarbon group,
n is an integer from 2 to 5,
m is an integer from 30 to 70, and
L is a linker,
the method comprising:
   a) contacting a secondary amine having a chemical formula of R¹—CH₂—NH—R² with an polyolefin-glycol compound to couple the secondary amine and the polyolefin-glycol compound to form the compound of Formula I.
Aspect 143 is directed to the method of aspect 142, wherein the polyolefin-glycol compound is a polyolefin glycol acid having a chemical formula of HOOC—L—(O(CH₂)ₙ)ₘ—OR³.
Aspect 144 is directed to the method of aspect 143, wherein the secondary amine and the polyolefin glycol acid have a molar ratio of 1:1.2 to 1:1.5.
Aspect 145 is directed to the method of any one of aspects 143 to 144, wherein the polyolefin glycol acid is activated by contacting the polyolefin glycol acid with an organic base and a coupling agent to form a coupling solution comprising an activated polyolefin-glycol compound, and the coupling solution is contacted with the secondary amine.
Aspect 146 is directed to the method of aspect 145, wherein the organic base is a tertiary amine.
Aspect 147 is directed to the method of aspect 146, wherein the tertiary amine is diisopropylethylamine.
Aspect 148 is directed to the method of any one of aspects 145 to 147, wherein the coupling agent is 1-propanephosphonic acid cyclic anhydride.
Aspect 149 is directed to the method of any one of aspects 145 to 148, wherein the coupling solution is colorless.
Aspect 150 is directed to the method of any one of aspects 145 to 149, wherein the coupling solution is formed by contacting the polyolefin glycol acid and the organic base at a molar ratio of 1:3.5 to 1:4.5.
Aspect 151 is directed to the method of any one of aspects 145 to 150, wherein the coupling solution is formed by contacting the polyolefin glycol acid and the coupling agent at a molar ratio of 1:1.8 to 1:2.2.
Aspect 152 is directed to the method of any one of aspects 145 to 151, wherein the coupling solution is formed by contacting the polyolefin glycol acid with an organic solvent to form a polyolefin glycol solution, distilling the polyolefin glycol solution to form a distilled polyolefin glycol solution, and contacting the distilled polyolefin glycol solution with the base and coupling agent to form the coupling solution.
Aspect 153 is directed to the method of aspect 152, wherein the polyolefin glycol solution is distilled at a pressure of 0 to 0.2 bar and/or a temperature at or below 70° C.
Aspect 154 is directed to the method of any one of aspects 152 to 153, wherein the distilled polyolefin glycol solution contains less than 0.05 wt. % of water.
Aspect 155 is directed to the method of any one of embodiments 152 to 154, wherein the polyolefin glycol acid is contacted with toluene to form the polyolefin glycol solution.
Aspect 156 is directed to the method of any one of aspects 142 to 155, wherein the compound of Formula I is formed at a temperature of 20° C. to 45° C.
Aspect 157 is directed to the method of aspect 142, wherein the polyolefin-glycol compound is a N-hydroxylsuccinimide (NHS) functionalized polyolefin glycol, and has a chemical formula of NHS—O(O)C—L—(O(CH₂)ₙ)ₘ—OR³.
Aspect 158 is directed to the method of aspect 157, wherein the NHS functionalized polyolefin glycol is contacted with secondary amine at a molar ratio 0.6:1 to 1.2:1.
Aspect 159 is directed to the method of any one of aspects 157 to 158, wherein the NHS functionalized polyolefin glycol is contacted with the secondary amine in the presence of a tertiary amine.
Aspect 160 is directed to the method of aspect, wherein the tertiary amine is triethylamine.
Aspect 161 is directed to the method of any one of aspects 157 to 160, wherein the NHS functionalized polyolefin glycol is contacted with the secondary amine at a temperature of 20° C. to 45° C.
Aspect 162 is directed to the method of any one of aspects 157 to 161, wherein the NHS functionalized polyolefin glycol is contacted with a reformed secondary amine.
Aspect 163 is directed to the method of any one of aspects 157 to 162, wherein the NHS functionalized polyolefin glycol is contacted with a reformed secondary amine from a distilled organic solution.
Aspect 164 is directed to the method of any one of aspects 142 to 163, wherein the method further comprises quenching the coupling reaction between the polyolefin-glycol compound with the secondary amine by adding an aqueous quench solution comprising potassium carbonate and sodium chloride.
Aspect 165 is directed to the method of aspect 164, wherein quenching the coupling comprises:
   contacting the aqueous quench solution with a coupling-product mixture to form a biphasic product mixture comprising i) an organic phase comprising the compound of Formula I and less than 10 wt. % of the secondary amine, and ii) an aqueous phase,
   separating the organic phase and the aqueous phase of the biphasic product mixture, and
   distilling the organic phase to form a product solution comprising the compound of Formula I and less than 0.12 wt. % of water.
Aspect 166 is directed to the method of aspect 165, wherein the organic phase is distilled at a pressure of 0 to 0.2 bar and/or a temperature at or below 70° C. to form the product solution.
Aspect 167 is directed to the method of any one of aspects 142 to 166, wherein the method further comprises at least partially purifying the compound of Formula I.
Aspect 168 is directed to the method of aspect 167, wherein the compound of Formula I is at least partially purified by silica gel chromatography or polymer resin chromatography.
Aspect 169 is directed to the method of any one of aspects 167 to 168, further comprising precipitating the compound of Formula I, the process comprising:
   obtaining an ethanol solution of the at least partially purified compound of Formula I,
   contacting isopropanol with the ethanol solution to form an isopropanol and ethanol mixture, wherein the compound of Formula I precipitates from the isopropanol and ethanol mixture, and
   separating the precipitate of the compound of Formula I from the isopropanol and ethanol mixture.
Aspect 170 is directed to the method of aspect 169, wherein isopropanol is contacted with the ethanol solution at an isopropanol:ethanol weight ratio of 3.5:1 to 2.5:1.
Aspect 171 is directed to the method of any one of aspects 169 to 170, wherein the precipitate of the compound of Formula I is separated from the isopropanol and ethanol mixture by filtration.
Aspect 172 is directed to the method of any one of aspects 142 to 171, wherein the n is 2 and m is 40 to 50.
Aspect 173 is directed to the method of any one of aspects 142 to 172, wherein R³ is an alkyl group.
Aspect 174 is directed to the method of any one of aspects 142 to 173, wherein R³ is a methyl group.
Aspect 175 is directed to the method of any one of aspects 142 to 174, wherein R¹ and R² are independently a linear, saturated, and unsubstituted alkyl group.
Aspect 176 is directed to the method of any one of aspects 142 to 175, wherein R¹ and R² independently have a chemical formula selected from the group —(CH₂)₇CH₃,—(CH₂)₈CH₃, —(CH₂)₉CH₃, —(CH₂)₁₀CH₃, —(CH₂)₁₁CH₃, —(CH₂)₁₂CH₃, —(CH₂)₁₃CH₃, —(CH₂)₁₄CH₃, —(CH₂)₁₅CH₃, —(CH₂)₁₆CH₃, —(CH₂)₁₇CH₃, —(CH₂)₁₈CH₃, and —(CH₂)₁₉CH₃.
Aspect 177 is directed to the method of any one of aspects 142 to 176, wherein R¹ is —(CH₂)₁₂CH₃ and/or R² is —(CH₂)₁₃CH₃ group.
Aspect 178 is directed to the method of any one of aspects 142 to 177, wherein L has a chemical formula of —(CH₂)ₐ—X—(CH₂)_{a″}—, wherein a′ and a″ are independently 0, 1, 2, 3, 4, or 5, and X is a linker.
Aspect 179 is directed to the method of aspect 178, wherein X is selected from a bond, —HC═CH—, —C≡C—, —C₆H₄—, —O—, or —S—.
Aspect 180 is directed to the method of any one of aspects 142 to 179, wherein L is —CH₂-.
Aspect 181 is directed to the method of any one of aspects 142 to 180, wherein Formula I is Formula Ia

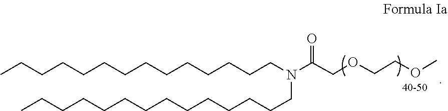

Aspect 182 is directed to a salt comprising a cation having the formula of Formula II

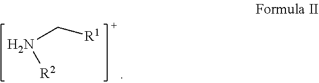

wherein R¹ and R² are independently a i) linear or branched or cyclic, ii) saturated or unsaturated, and iii) substituted or unsubstituted hydrocarbon group comprising 8 to 20 carbon atoms, and
an anion selected from chloride, bromide, iodide, sulfate, acetate, mesylate, tosylate, (1R)-(-)-10-camphorsulfonate, 1,2-ethanedisulfonate, oxalate, dibenzoyl-L-tartarate, phosphate, L-tartarate, maleate, fumarate, succinate, and malonate.
Aspect 183 is directed to the salt of aspect 182, wherein the anion is succinate.
Aspect 184 is directed to the salt of any one of aspects 182 to 183, wherein R¹ and R are independently a linear, saturated, and unsubstituted alkyl group.
Aspect 185 is directed to the salt of any one of aspects 182 to 184, wherein R¹ is a linear, saturated, and unsubstituted alkyl group containing 13 carbon atoms, and/or R² is a linear, saturated, and unsubstituted alkyl group containing 14 carbon atoms.
Aspect 186 is directed to the salt of any one of aspects 182 to 185, wherein Formula II is Formula IIa

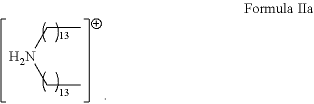

Aspect 187 is directed to the salt of any one of aspects 182 to 186, wherein the salt is in a crystallized form.
Aspect 188 is directed to a method for producing the salt in a crystallized form of any one of embodiments 182 to 187, the method comprising:
   a) contacting an amide having a chemical formula of R¹—C(O)—NH—R² with a reducing agent to form a secondary amine having a chemical formula of R¹—CH₂—NH—R²; and
   b) forming the salt in a crystallized form.
Aspect 189 is directed to the method of aspect 188, wherein the reducing agent is a hydride.
Aspect 190 is directed to the method of any one of aspect 189, wherein the hydride is lithium aluminum hydride.
Aspect 191 is directed to the method of any one of aspects 188 to 190, wherein the amide and the reducing agent have a molar ratio of 1:1 to 1:3.
Aspect 192 is directed to the method of any one of aspects 188 to 191, wherein when the amide and reducing agent are contacted, the amide is comprised in an amide solution and the reducing agent is comprised in a reducing agent solution.
Aspect 193 is directed to the method of aspect 192, wherein the amide solution further comprises toluene, and/or the reducing agent solution further comprises tetrahydrofuran (THF) and/or 2-methyl THF.
Aspect 194 is directed to the method of any one of aspects 192 to 193, wherein the amide solution is formed by contacting crystals of the amide with toluene.
Aspect 195 is directed to the method of any one of aspects 188 to 194, wherein the secondary amine is formed at a temperature of 50° C. to 75° C.
Aspect 196 is directed to the method of any one of aspects 188 to 195, wherein in step (a) the amide is reduced, and step (a) further comprises quenching the reduction of the amide by adding sodium sulfate.
Aspect 197 is directed to the method of aspect 196, wherein quenching the reduction comprises:
   contacting a reduction-product mixture comprising the secondary amine formed in step (a) with a slurry comprising sodium sulfate at a temperature of 35° C. to 45° C. to form a quenched reduction-product mixture and residual sodium sulfate;
   separating at least a portion of residual sodium sulfate from the quenched reduction-product mixture to form a separated reduction-product mixture comprising the secondary amine.
Aspect 198 is directed to the method of aspects 197, wherein the reduction-product mixture comprises less than 4% by weight of the starting amide.
Aspect 199 is directed to the method of any one of aspects 196 to 198, wherein 0.5 to 2 moles of sodium sulfate per mole amide is added.
Aspect 200 is directed to the method of any one of aspects 197 to 199, wherein the slurry comprising sodium sulfate further comprises THF.
Aspect 201 is directed to the method of any one of aspects 197 to 200, wherein the at least a portion of the residual sodium sulfate is separated from the quenched reduction-product mixture by filtration, wherein the separated reduction-product mixture is formed as a filtrate.
Aspect 202 is directed to the method of any one of aspects 188 to 201, wherein forming the salt in crystalized form comprises,
   contacting the secondary amine with an acid to form a salt-forming solution comprising a salt of the secondary amine, and
   cooling the salt-forming solution to form the salt in crystallized form.
Aspect 203 is directed to the method of any one of aspects 188 to 202, wherein forming the salt in crystalized form comprises:
   contacting the secondary amine with isopropanol and an acid at a temperature of 50° C. to 60° C. to form a salt-forming solution comprising a salt of the secondary amine;
   cooling the salt-forming solution to 30° C. to 45° C. to form salt crystals; maintaining the salt-forming solution at 30° C. to 45° C. for at least 1 hour;
   cooling the salt-forming solution to 15° C. to 25° C.;
   separating the salt crystals from the salt-forming solution.
Aspect 204 is directed to the method of any one of aspects, wherein the salt crystals are separated from the salt-forming solution by filtering, wherein the salt in crystallized form is obtained as a filtered residue.
Aspect 205 is directed to the method of aspect 204, further comprising washing and drying the filtered residue to form a dried, crystallized salt.
Aspect 206 is directed to the method of aspect 205, wherein the filtered residue is washed with a toluene and/or isopropanol solution.
Aspect 207 is directed to the method of aspect 206, wherein volume % ratio of the toluene and isopropanol in the toluene and/or isopropanol solution is 0.9:1 to 1:0.9.
Aspect 208 is directed to the method of any one of aspects 205 to 207, wherein the filtered residue is dried at a pressure of 0 to 0.2 bar and/or a temperature of 40° C. to 50° C.
Aspect 209 is directed to the method of any one of aspects 202 to 208, wherein the acid is hydrochloric acid, hydrobromic acid, hydroiodic acid, sulfuric acid, acetic acid, methanesulfonic acid, toluenesulfonic acid, (1R)-(-)-10-camphorsulfonic acid, 1,2-ethanedisulfonic acid, oxalic acid, dibenzoyl-L-tartaric acid, phosphoric acid, L-tartaric acid, maleate, fumaric acid, succinic acid, and/or malonic acid.
Aspect 210 is directed to the method of any one of aspect 202 to 209, wherein the acid is succinic acid.
Aspect 211 is directed to a method for producing a compound having a chemical formula of Formula I

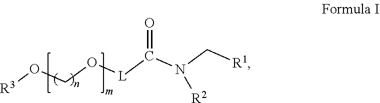

wherein R¹ and R² are independently a i) linear or branched or cyclic, ii) saturated or unsaturated, and iii) substituted or unsubstituted hydrocarbon group comprising 8 to
20 carbon atoms,
R³ is a hydrocarbon group,
n is an integer from 2 to 5,
n is an integer from 30 to 70, and
L is a linker,
the method comprising:
   a) contacting a fatty acid having a chemical formula of R¹—COOH, with 1,1′-Carbonyldiimidazole (CDI) to form a N-acyl imidazole having the chemical formula of R¹—C(O)—C₃N₂H₄,
   b) contacting the N-acyl imidazole with a primary amine having a chemical formula of R²—NH₂ to form an amide having a chemical formula of R¹—C(O)—NH—
   c) contacting the amide with a reducing agent to form a secondary amine having a chemical formula of R¹—CH₂—NH—R²; and
   d) contacting the secondary amine with a polyolefin-glycol compound to form the compound of Formula I,
      wherein the fatty acid and CDI have a molar ratio of 1:1.2 to 1.2:1,
      wherein contacting the fatty acid with CDI is performed at a temperature of 40° C. to 60° C.,
      wherein the N-acyl imidazole and the primary amine have a molar ratio of 0.9:1 to 1:0.9,
      wherein the amount of primary amine contacted with the N-acyl imidazole is 0.85 to 1.2 moles of primary amine per mole of the fatty acid used to form the N-acyl imidazole,
      wherein the N-acyl imidazole and the primary amine are contacted at a temperature of 40° C. to 60° C.,
      wherein the reducing agent is a hydride, such as lithium aluminum hydride,
      wherein the amide and the reducing agent is contacted at a temperature of 50° C. to 75° C.,
      wherein the polyolefin-glycol compound is a polyolefin glycol acid having a chemical formula of HOOC—L—(O(CH₂)ₙ)ₘ—OR³,
      wherein the secondary amine and the polyolefin glycol acid have a molar ratio of 1:1.2 to 1:1.5,
      wherein the polyolefin glycol acid is activated by contacting the polyolefin glycol acid with an organic base such as diisopropylethylamine, and a coupling agent such as 1-propanephosphonic acid cyclic anhydride, to form a coupling solution comprising an activated polyolefin-glycol compound, and the coupling solution is contacted with the secondary amine,
      wherein the coupling solution is formed by contacting the polyolefin glycol acid and the organic base at a molar ratio of 1:3.5 to 1:4.5,
      wherein the coupling solution is formed by contacting the polyolefin glycol acid and the coupling agent at a molar ratio of 1:1.8 to 1:2.2, and
      wherein the compound of Formula I is formed at a temperature of 20° C. to 45° C.
Aspect 212 is directed to a method for producing a compound having a chemical formula of Formula I

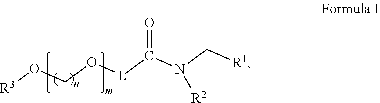

wherein R¹ and R² are independently a i) linear or branched or cyclic, ii) saturated or unsaturated, and iii) substituted or unsubstituted hydrocarbon group comprising 8 to
20 carbon atoms,
R³ is a hydrocarbon group,
n is an integer from 2 to 5,
m is an integer from 30 to 70, and
L is a linker,
the method comprising:
   a) contacting a fatty acid having a chemical formula of R¹—COOH, with an oxychloride to form an acyl chloride having a chemical formula of R¹—C(O)—Cl, b) contacting the acyl chloride with a primary amine having a chemical formula of R²—NH₂ to form an amide having a chemical formula of R¹—C(O)—NH—R²;
   c) contacting the amide with a reducing agent to form a secondary amine having a chemical formula of R¹—CH₂—NH—R²; and
   d) contacting the secondary amine with a polyolefin-glycol compound to form the compound of Formula I,
      wherein the oxychloride is selected from thionyl chloride, phosphoryl chloride, oxalyl chloride, and any combinations thereof,
      wherein the fatty acid and the oxychloride have a molar ratio of 1:0.8 to 1:2,
      wherein the fatty acid and the oxychloride are contacted at a temperature of 20° C. to 75° C.,
      wherein the acyl chloride and primary amine are contacted in the presence of benzene and triethylamine,
      wherein the amount of primary amine contacted with the acyl chloride is 0.6 to 1.2 moles of primary amine per mole of the fatty acid used to form the acyl chloride,
      wherein the reducing agent is a hydride, such as lithium aluminum hydride,
      wherein the amide and the reducing agent is contacted at a temperature of 50° C. to 75° C.,
      wherein the polyolefin-glycol compound is a polyolefin glycol acid having a chemical formula of HOOC—L—(O(CH₂)ₙ)ₘ—OR³,
      wherein the secondary amine and the polyolefin glycol acid have a molar ratio of 1:1.2 to 1:1.5,
      wherein the polyolefin glycol acid is activated by contacting the polyolefin glycol acid with an organic base such as diisopropylethylamine, and a coupling agent such as 1-propanephosphonic acid cyclic anhydride, to form a coupling solution comprising an activated polyolefin-glycol compound, and the coupling solution is contacted with the secondary amine,
      wherein the coupling solution is formed by contacting the polyolefin glycol acid and the organic base at a molar ratio of 1:3.5 to 1:4.5,
      wherein the coupling solution is formed by contacting the polyolefin glycol acid and the coupling agent at a molar ratio of 1:1.8 to 1:2.2, and
      wherein the compound of Formula I is formed at a temperature of 20° C. to 45° C.
Aspect 213 is directed to a method for producing a compound having a chemical formula of Formula I

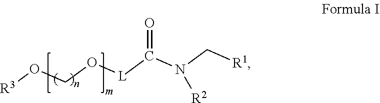

wherein R¹ and R² are independently a i) linear or branched or cyclic, ii) saturated or unsaturated, and iii) substituted or unsubstituted hydrocarbon group comprising 8 to
20 carbon atoms,
R³ is a hydrocarbon group,
n is an integer from 2 to 5,
m is an integer from 30 to 70, and
L is a linker,
the method comprising:
   a) contacting a fatty acid having a chemical formula of R¹—COOH, with 1,1′-Carbonyldiimidazole (CDI) to form a N-acyl imidazole having the chemical formula of R—C(O)—C₃N₂H₄,
   b) contacting the N-acyl imidazole with a primary amine having a chemical formula of R²—NH₂ to form an amide having a chemical formula of R¹—C(O)—NH—R²
   c) contacting the amide with a reducing agent to form a secondary amine having a chemical formula of R¹—CH₂—NH—R²; and
   d) contacting the secondary amine with a polyolefin-glycol compound to form the compound of Formula I,
      wherein the fatty acid and CDI have a molar ratio of 1:1.2 to 1.2:1,
      wherein contacting the fatty acid with CDI is performed at a temperature of 40° C. to 60° C.,
      wherein the N-acyl imidazole and the primary amine have a molar ratio of 0.9:1 to 1:0.9,
      wherein the amount of primary amine contacted with the N-acyl imidazole is 0.85 to 1.2 moles of primary amine per mole of the fatty acid used to form the N-acyl imidazole,
      wherein the N-acyl imidazole and the primary amine are contacted at a temperature of 40° C. to 60° C.,
      wherein the reducing agent is a hydride, such as lithium aluminum hydride,
      wherein the amide and the reducing agent is contacted at a temperature of 50° C. to 75° C.,
      wherein the polyolefin-glycol compound is a N-hydroxylsuccinimide (NHS) functionalized polyolefin glycol, and has a chemical formula of NHS—O(O)C—L—(O(CH₂)ₙ)ₘ—OR³,
      wherein the NHS functionalized polyolefin glycol is contacted with the secondary amine at a molar ratio 0.6:1 to 1.2:1,
      wherein the NHS functionalized polyolefin glycol is contacted with the secondary amine in presence of a tertiary amine, such as trimethylamine,
      wherein the NHS functionalized polyolefin glycol is contacted with the secondary amine at a temperature of 20° C. to 45° C.
Aspect 214 is directed to a method for producing a compound having a chemical formula of Formula I

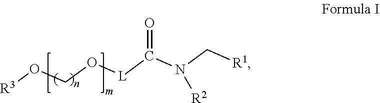

wherein R¹ and R² are independently a i) linear or branched or cyclic, ii) saturated or unsaturated, and iii) substituted or unsubstituted hydrocarbon group comprising 8 to
20 carbon atoms,
R³ is a hydrocarbon group,
n is an integer from 2 to 5,
m is an integer from 30 to 70, and
L is a linker,
the method comprising:
   a) contacting a fatty acid having a chemical formula of R¹—COOH, with an oxychloride to form an acyl chloride having a chemical formula of R¹—C(O)—Cl,
   b) contacting the acyl chloride with a primary amine having a chemical formula of R²—NH₂ to form an amide having a chemical formula of R¹—C(O)—NH—R²;
   c) contacting the amide with a reducing agent to form a secondary amine having a chemical formula of R¹—CH₂—NH—R²; and
   d) contacting the secondary amine with a polyolefin-glycol compound to form the compound of Formula I,
      wherein the oxychloride is selected from thionyl chloride, phosphoryl chloride, oxalyl chloride, and any combinations thereof,
      wherein the fatty acid and the oxychloride have a molar ratio of 1:0.8 to 1:2,
      wherein the fatty acid and the oxychloride are contacted at a temperature of 20° C. to 75° C.,
      wherein the acyl chloride and primary amine are contacted in the presence of benzene and triethylamine,
      wherein the amount of primary amine contacted with the acyl chloride is 0.6 to 1.2 moles of primary amine per mole of the fatty acid used to form the acyl chloride,
      wherein the reducing agent is a hydride, such as lithium aluminum hydride,
      wherein the amide and the reducing agent is contacted at a temperature of 50° C. to 75° C.,
      wherein the polyolefin-glycol compound is a N-hydroxylsuccinimide (NHS) functionalized polyolefin glycol, and has a chemical formula of NHS—O(O)C—L—(O(CH₂)ₙ)ₘ—OR³,
      wherein the NHS functionalized polyolefin glycol is contacted with the secondary amine at a molar ratio 0.6:1 to 1.2:1,
      wherein the NHS functionalized polyolefin glycol is contacted with the secondary amine in presence of a tertiary amine, such as trimethylamine,
      wherein the NHS functionalized polyolefin glycol is contacted with the secondary amine at a temperature of 20° C. to 45° C.

The following includes definitions of various terms and phrases used throughout this specification.

As used herein, the term “about,” or “approximately” is used to indicate that a value includes the standard deviation of error for the device or method being employed to determine the value. In some aspects, the term “about” can be added to any numeral recited herein to the extent the numeral would have a standard deviation of error when measuring.

The terms “wt. %,” “vol. %,” or “mol. %” refers to a weight percentage of a component, a volume percentage of a component, or molar percentage of a component, respectively, based on the total weight, the total volume of material, or total moles, that includes the component. In a non-limiting example, 10 grams of component in 100 grams of the material is 10 wt. % of component.

The term “substantially” and its variations are defined to include ranges within 10%, within 5%, within 1%, or within 0.50%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having” in the claims, or the specification, may mean “one,” but it is also consistent with the meaning of “one or more,” “at least one,” and “one or more than one.”

The phrase “and/or” means and or or. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions, process, and systems disclosed by the Applicant herein can “comprise,” “consist essentially of,” or “consist of” particular ingredients, components, compositions, steps, etc. disclosed throughout the specification.

The term “hydrocarbon” as used herein refer to alkyl, heteroalkyl, cycloalkyl, aryl, and heteroaryl groups. The groups (e.g., alkyl, heteroalkyl, cycloalkyl, aryl, and heteroaryl) can be substituted or unsubstituted, saturated or unsaturated, branched or unbranched, cyclic or acyclic.

The term “alkyl,” by itself or as part of another substituent, means, unless otherwise stated, a linear (i.e., unbranched) or branched carbon chain, which may be fully saturated, monounsaturated, or polyunsaturated. An unsaturated alkyl groups include those having one or more carbon-carbon double bonds (alkenyl) and those having one or more carbon-carbon triple bonds (alkynyl). The groups, —CH₃ (Me), —CH₂CH₃ (Et), —CH₂CH₂CH₃ (n-Pr), —CH(CH₃)₂ (iso-Pr), —CH₂CH₂CH₂CH₃ (n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), are all non-limiting examples of alkyl groups.

The term “heteroalkyl” or “substituted alkyl,” by itself or in combination with another term, means, unless otherwise stated, a linear or branched chain having at least one carbon atom and at least one heteroatom. The heteroatom in some instances can be selected from the group consisting of one or more of F, Cl, Br, I, O, N, S, P, and Si. In certain aspects, the heteroatoms are selected from the group consisting of one or more of O and N. The heteroatom(s) may be placed at any interior position, terminal of the heteroalkyl group, or at the position at which the alkyl group is attached to the remainder of the molecule. Up to two heteroatoms may be consecutive. The following groups are all non-limiting examples of heteroalkyl groups: trifluoromethyl, —CH₂ F, —CH₂ Cl, —CH₂ Br, —CH₂ OH, —CH₂ OCH₃, —CH₂ OCH₂ CF₃, —CH₂OC(O)CH₃, —CH₂ NH₂, —CH₂ NHCH₃, —CH₂ N(CH₃)₂, —CH₂CH₂Cl, —CH₂CH₂OH, CH₂CH₂OC(O)CH₃, —CH₂CH₂ NHCO₂C(CH₃)₃, and —CH₂ Si(CH₃)₃. The heteroakyl group can be saturated or unsaturated.

The terms “cycloalkyl” and “heterocyclyl,” by themselves or in combination with other terms, means cyclic versions of “alkyl” and “heteroalkyl”, respectively. Additionally, for heterocyclyl, a heteroatom can occupy the position at which the heterocycle is attached to the remainder of the molecule.

The term “aryl” means a polyunsaturated, aromatic, hydrocarbon substituent. Aryl groups can be monocyclic or polycyclic (e.g., 2 to 3 or more rings that are fused together or linked covalently). The term “heteroaryl” refers to an aryl group that contains one or more heteroatoms selected from N, O, and S. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

As described herein a “substituted” or a “substituted group” can refer to groups that include one or more substituents independently selected from: halogen, nitro, cyano, hydroxy, amino, mercapto, formyl, carboxy, oxo, carbamoyl, substituted or unsubstituted alkyl, substituted or unsubstituted heteroalkyl, alkoxy, alkylthio, alkylamino, (alkyl)₂amino, alkylsulfinyl, alkylsulfonyl, arylsulfonyl, substituted or unsubstituted cycloalkyl, substituted or unsubstituted heterocyclyl, substituted or unsubstituted aryl, and substituted or unsubstituted heteroaryl. In certain aspects the substituents may be further substituted with one or more substituents independently selected from: halogen, nitro, cyano, hydroxy, amino, mercapto, s formyl, carboxy, carbamoyl, unsubstituted alkyl, unsubstituted heteroalkyl, alkoxy, alkylthio, alkylamino, (alkyl)₂amino, alkylsulfinyl, alkylsulfonyl, arylsulfonyl, unsubstituted cycloalkyl, unsubstituted heterocyclyl, unsubstituted aryl, or unsubstituted heteroaryl. Exemplary substituents include, but are not limited to: —OH, oxo (═O), —Cl, —F, Br, C₁₋₄alkyl, phenyl, benzyl, —NH₂, —NH(C₁₋₄alkyl), —N(C₁₋₄alkyl)₂, —NO₂, —S(C₁₋₄alkyl), —SO₂(C₁₋₄alkyl), —CO₂(C₁₋₄alkyl), and —O(C₁₋₄alkyl).

The term “alkoxy” means a group having the structure —OR′, where R′ is an optionally substituted alkyl or cycloalkyl group. The term “heteroalkoxy” similarly means a group having the structure —OR, where R is a heteroalkyl or heterocyclyl.

The term “amino” means a group having the structure —NR′R″, where R′ and R″ are is independently hydrogen or an optionally substituted alkyl, heteroalkyl, cycloalkyl, or heterocyclyl group. The term “amino” includes primary, secondary, and tertiary amines.

The term “oxo” as used herein means an oxygen that is double bonded to a carbon atom.

The term “alkylsulfonyl” as used herein means a moiety having the formula —S(O₂)—R′, where R′ is an alkyl group. R′ may have a specified number of carbons (e.g., “C₁₋₄ alkylsulfonyl”).

As used herein, the term “nitro” means —NO₂; the term “halo” designates —F, —Cl, —Br or —I; the term “mercapto” means —SH; the term “cyano” means —CN; the term “azido” means —N₃; the term “silyl” means —SiH₃, and the term “hydroxyl” means —OH.

The term “pharmaceutically acceptable salts,” as used herein, refers to salts of compounds that are substantially non-toxic to living organisms. Typical pharmaceutically acceptable salts include those salts prepared by reaction of a compound with an inorganic or organic acid, or an organic base, depending on the substituents present on the compounds.

Non-limiting examples of inorganic acids which may be used to prepare pharmaceutically acceptable salts include or can exclude: hydrochloric acid, phosphoric acid, sulfuric acid, hydrobromic acid, hydroiodic acid, phosphorous acid and the like. Examples of organic acids which may be used to prepare pharmaceutically acceptable salts include or can exclude: aliphatic mono- and dicarboxylic acids, such as oxalic acid, carbonic acid, citric acid, succinic acid, phenyl-heteroatom-substituted alkanoic acids, aliphatic and aromatic sulfuric acids and the like. Pharmaceutically acceptable salts prepared from inorganic or organic acids thus include or can exclude hydrochloride, hydrobromide, nitrate, sulfate, pyrosulfate, bisulfate, sulfite, bisulfate, phosphate, monohydrogenphosphate, dihydrogenphosphate, metaphosphate, pyrophosphate, hydroiodide, hydro fluoride, acetate, propionate, formate, oxalate, citrate, lactate, p-toluenesulfonate, methanesulfonate, maleate, and the like.

Suitable pharmaceutically acceptable salts may also be formed by reacting compounds with an organic base such as methylamine, ethylamine, ethanolamine, lysine, ornithine and the like. Pharmaceutically acceptable salts include or can exclude the salts formed between carboxylate or sulfonate groups found on some of the compounds disclosed by the Applicant herein and inorganic cations, such as sodium, potassium, ammonium, or calcium, or such organic cations as isopropylammonium, trimethylammonium, tetramethylammonium, and imidazolium.

It should be recognized that the particular anion or cation forming a part of any salt of the compounds disclosed by the Applicant herein in some instances is not critical, so long as the salt, as a whole, is pharmacologically acceptable. However, in some instances, use of particular salts can provide benefits, such as increased or decreased solubility in certain solvents or bioavailability, increased ability to remove or retain the anion or cation in downstream steps, increased safety for administration to a subject, decrease in environmentally dangerous waste, and/or increased environmental safety of the intermediates and/or final products.

Additional examples of pharmaceutically acceptable salts and their methods of preparation and use are presented in Handbook of Pharmaceutical Salts: Properties, Selection and Use (2002), which is incorporated herein by reference.

Other objects, features and advantages of the present invention will become apparent from the following figures, detailed description, and examples. It should be understood, however, that the figures, detailed description, and examples, while indicating specific embodiments and/or aspects of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments and/or aspects may be combined with features from other aspects. For example, features from one embodiment and/or aspect may be combined with features from any of the other embodiments and/or aspects. In further aspects, additional features may be added to the specific aspects described herein.

### DESCRIPTION

Methods for producing polymer conjugated lipids and intermediates for the production thereof are described. The method can include forming the polymer conjugated lipid via intermediate formation of an amide and a secondary amine. In some instances, the amount of time needed to produce the final product and/or intermediates for the production thereof is shortened in comparison to that previously achieved, due to one or more reaction steps using different reagents and/or reaction conditions than those used previously to produce a polymer conjugated lipid. In some instances, the amount of time needed to produce the final product and/or intermediates for the production thereof is shortened in comparison, due to not needing solvent lyophilization to isolate the final product and/or intermediates in solid form. In certain aspects, the method includes formation of solid crystals and/or solid precipitate of an intermediate, salts of an intermediate, and/or final product, without solvent lyophilization. Solvent lyophilization to isolate a product in solid form may require extensive time and energy. Crystallization of the intermediate and/or salts of an intermediate can increase overall purity of the final product. Forming the final product in solid form can provide for easier handling, storage, and transportation of the final product. In another aspect, salts of the polymer conjugated lipids and intermediates for the production thereof are disclosed. In some instances, the salts can be pharmaceutically acceptable, be environmentally safe, and/or have improved solubility or insolubility, bioavailability, purity, and/or steps for removal and/or replacement of the salt.

In some preferred embodiments, methods for synthesis of a polymer-conjugated lipid exemplified herein begins with two starting materials, tetradecane-1-amine and myristic acid, which are converted to an amide using carbonyldiimidazole in toluene. The product is reduced using lithium aluminum hydride in toluene, and the N-tetamine is isolated as its succinic acid salt to increase purity, which simplified downstream processing. Tetamine is pegylated through an amidation reaction using T3P in toluene. After chromatography, the pure polymer-conjugated lipid was isolated as a solid. The streamlined process showed significant Green Chemistry improvement, among other aspects, with a 3-fold reduction of the Process Mass Intensity (PMI) (˜900 Kg/Kg of ALC-159) and elimination of chloroform from the process.

These and other non-limiting aspects of the present invention are discussed in further detail in the following sections.

### I. Compounds Having Chemical Formula of Formula I

Certain aspects are directed to methods for producing a compound having the chemical formula of Formula I. The compound of Formula I can form a polymer conjugated lipid.

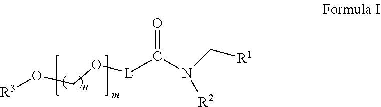

R¹ and R² can independently be a hydrocarbon group containing 8, 9, 10, 11, 12, 13, 14, 15, 16, 17, 18, 19, or 20 carbon atoms. In certain aspects, R¹ and R² are independently a i) linear or branched or cyclic, ii) saturated or unsaturated, and iii) substituted or unsubstituted hydrocarbon group containing 8, 9, 10, 11, 12, 13, 14, 15, 16, 17, 18, 19, or 20 carbon atoms. In certain aspects, R¹ and/or R² are independently a linear, saturated, substituted alkyl group. In certain aspects, R¹ and/or R² are independently a linear, saturated, unsubstituted alkyl group. In certain aspects, R^{l} and/or R² are independently a linear, unsaturated, substituted alkyl group. In certain aspects, R¹ and/or R² are independently a linear, unsaturated, unsubstituted alkyl group. In certain aspects, R¹ and/or R² are independently a branched, saturated, substituted 20 alkyl group. In certain aspects, R¹ and/or R² are independently a branched, saturated, unsubstituted alkyl group. In certain aspects, R¹ and/or R² are independently a branched, unsaturated, substituted alkyl group. In certain aspects, R¹ and/or R² are independently a branched, unsaturated, unsubstituted alkyl group. In certain aspects, R¹ and R² are independently a linear, saturated, unsubstituted alkyl group. In some particular aspects, R¹ and 25 R² are independently a linear, saturated, unsubstituted alkyl group containing 8, 9, 10, 11, 12, 13, 14, 15, 16, 17, 18, 19, or 20 carbon atoms. In some particular aspects, R¹ and R² independently have a chemical formula selected from the group —(CH₂)₇CH₃, —(CH₂)₈CH₃, —(CH₂)₉CH₃, —(CH₂)₁₀CH₃, —(CH₂)₁₁CH₃, —(CH₂)₁₂CH₃, —(CH₂)₁₃CH₃, —(CH₂)₁₄CH₃, —(CH₂)₁₅CH₃, —(CH₂)₁₆CH₃, —(CH₂)₁₇CH₃, —(CH₂)₁₈CH₃ and —(CH₂)₁₉CH₃. In some particular aspects, R¹ is —(CH₂)₁₂CH₃, and/or R² is —(CH₂)₁₃CH₃.

In certain aspects, R¹ and R² are the same. In certain aspects, R¹ and R² are different. In certain aspects, R¹ contains N carbon atoms and R² contains N+1 carbon atoms, where N is an integer from 8 to 19. In certain aspects, R¹ is a linear, saturated, unsubstituted alkyl group containing 13 carbon atoms. In certain aspects, R² is a linear, saturated, unsubstituted alkyl group containing 14 carbon atoms.

In certain aspects, m is 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70. In some particular aspects, m is 40, 41, 42, 43, 44, 45, 46, 47, 48, 49, or 50. In some particular aspects, m is 44. In certain aspects, n is 2, 3, 4, or 5. In some particular aspects, n is 2.

R³ can be a i) substituted or unsubstituted, ii) linear, branched, or cyclo, and iii) saturated or unsaturated hydrocarbon group. In certain aspects, R³ is a i) substituted or unsubstituted, ii) linear, or branched, or cyclo and iii) saturated or unsaturated alkyl group containing 1, 2, 3, 4, 5, 6, 7, 8, 9, 10, 11, 12, 13, 14, or 15 carbon atoms. In some aspects, R³ is a i) unsubstituted, ii) linear, or branched, and iii) saturated alkyl group containing 1, 2, 3, 4, 5, 6, 7, 8, 9, or 10 carbon atoms. In certain aspects, R³ is a methyl, ethyl, propyl, or butyl group. In some particular aspects, R³ is a methyl group.

L is a linker, and can have a chemical formula of —(CH₂)^{a′}—X— (CH₂)_{a″}—, where a′ and a″ can independently be an integer. In some aspects, a′ and a″ are independently 0, 1, 2, 3, 4, or 5. X can be a linker. In some aspects, X is selected from a bond, —HC═CH—, —C≡C—, —C₆H₄—

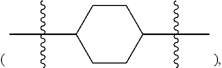 [SMILES: *C1CCC(*)CC1]

—O—, or —S—. In some aspects, L is —CH₂—, —(CH₂)₂—, or —(CH₂)₃—. In some particular aspects, L is —CH₂—.

In certain aspects, the compound of Formula I has the structure of any one of Formula (3) to (19):

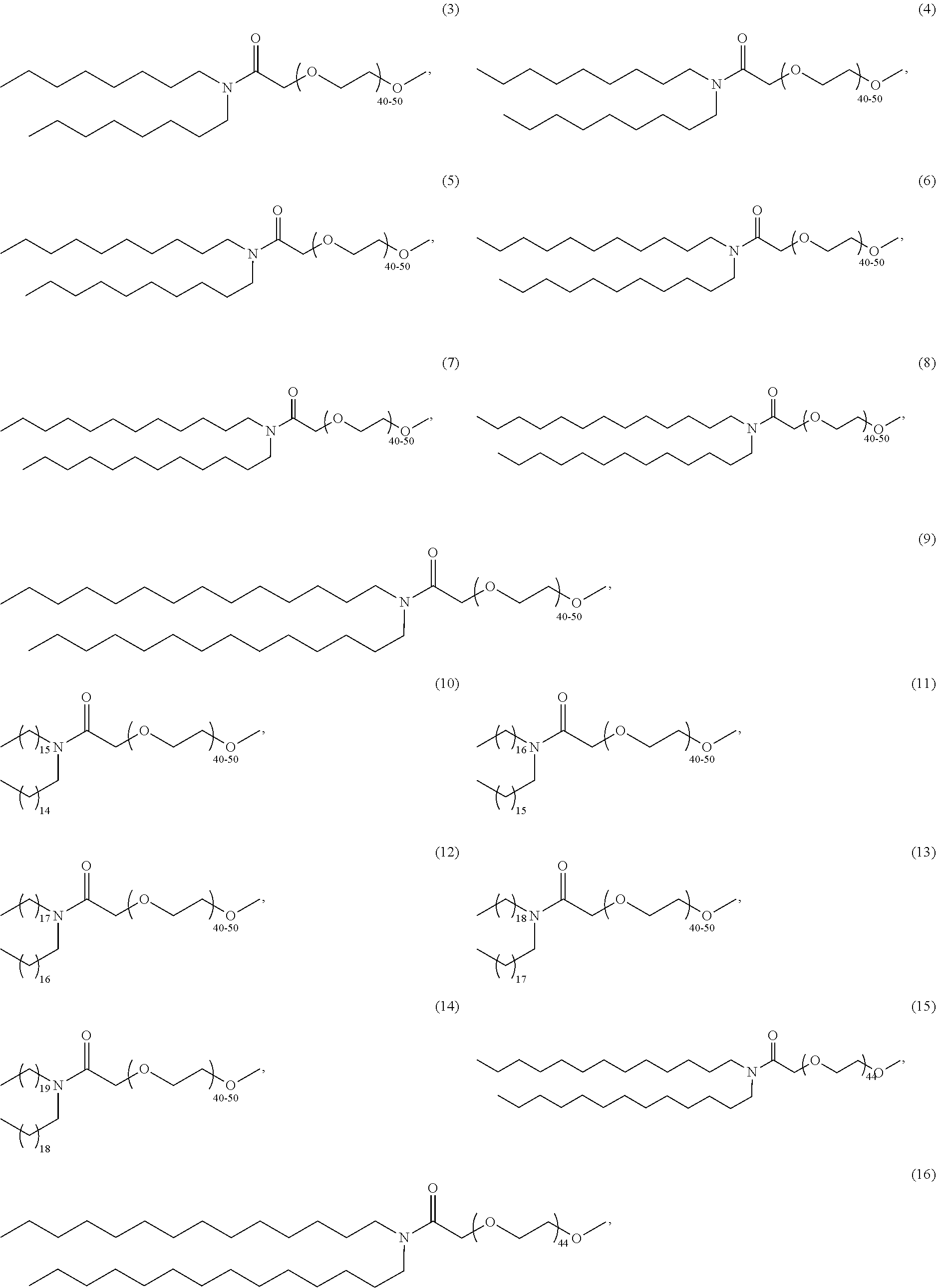

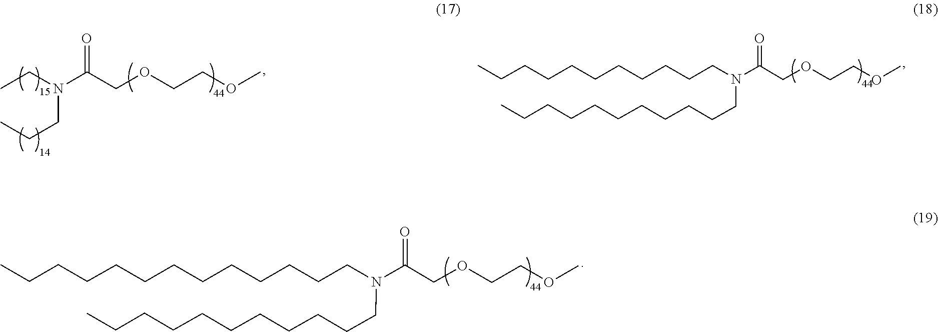

In some particular aspects, the compound of Formula I has the structure of Formula (16). In certain aspects, one or more compounds of Formula I described herein are excluded. 10 In certain aspects, one or more of R¹ groups described herein are excluded. In certain aspects, one or more of R² groups described herein are excluded. In certain aspects, one or more of R³ groups described herein are excluded. In certain aspects, one or more of n values described herein are excluded. In certain aspects, one or more m values described herein are excluded. In certain aspects, one or more L groups described herein are excluded.

### II. Methods of Preparing Compounds of Formula I, Intermediates Thereof and Salts of the Intermediate.

Certain aspects are directed to a method for preparing the compound of Formula I. The compounds of Formula I can be prepared by i) forming an amide having a chemical formula of R¹—C(O)—NH—R² from a fatty acid having a chemical of R¹—COOH and a primary amine having a chemical formula of R²—NH₂; ii) contacting the amide with a reducing agent to form a secondary amine having a chemical formula of R¹—CH₂—NH—R²; and iv) contacting the secondary amine with an polyolefin-glycol compound to form the compound of Formula I.

Certain aspects are directed to a polymer conjugated lipid described herein, an intermediate for the production thereof (e.g., the N-acyl imidazole acyl-chloride, amide, and/or secondary amine), a pharmaceutically acceptable salt of the lipid, and/or pharmaceutically acceptable salt of the intermediate. Certain aspects are directed to a composition containing a polymer conjugated lipid described herein, an intermediate for the production thereof (e.g., the N-acyl imidazole, acyl-chloride, amide, and/or secondary amine), a pharmaceutically acceptable salt of the lipid, and/or pharmaceutically acceptable salt of the intermediate, wherein the lipid and the intermediate is synthesized with a method described herein. In certain aspects, the composition contains a lipid having the structure of Formula (16), or a pharmaceutically acceptable salt thereof. Certain aspects, are directed to a use of a polymer conjugated lipid described herein, an intermediate for the production thereof (e.g., the N-acyl imidazole, acyl-chloride, amide, and/or secondary amine), a pharmaceutically acceptable salt of the lipid, and/or pharmaceutically acceptable salt of the intermediate.

### A. Formation of the Amide.

The amide can be formed using the fatty acid and the primary amine.

### 1. Formation of the Amide Via N-Acyl Imidazole.

In some aspects, the fatty acid is contacted with 1,1′-Carbonyldiimidazole (CDI) to form a N-acyl imidazole having a chemical formula of R¹—C(O)—C₃N₂H₄, and the N-acyl imidazole is contacted with the primary amine to form the amide.

### a) Formation of the N-Acyl Imidazole.

The N-acyl imidazole can be formed by a reaction between the fatty acid and CDI according to Scheme Ia. In certain aspects, the fatty acid and the CDI have, e.g., are contacted at, a molar ratio equal to any one of, at least any one of, at most any one of, or between any two of 1:1.2, 1.05:1.15, 1.1:1.1, 1.15:1.05, and 1.2:1 (or any range derivable therein). In certain aspects, a stoichiometric excess of the fatty acid is used. In certain aspects, contacting of the fatty acid with CDI is performed at a temperature equal to any one of, at least any one of, at most any one of, or between any two of 40, 41, 42, 43, 44, 45, 46, 47, 48, 49, 50, 51, 52, 53, 54, 55, 56, 57, 58, 59, and 60° C. (or any range derivable therein). In some aspects, the contact/reaction temperature is not higher than 60° C., or higher than 65° C., or higher than 70° C. In certain aspects, the fatty acid and CDI are contacted in the presence of an organic solvent, such as toluene. In certain aspects, a fatty acid solution containing the fatty acid, is contacted with a CDI solution and/or slurry containing CDI. In certain aspects, the fatty acid solution further contains an organic solvent, such as toluene. In certain aspects, the CDI solution and/or slurry further contains an organic solvent, such as toluene.

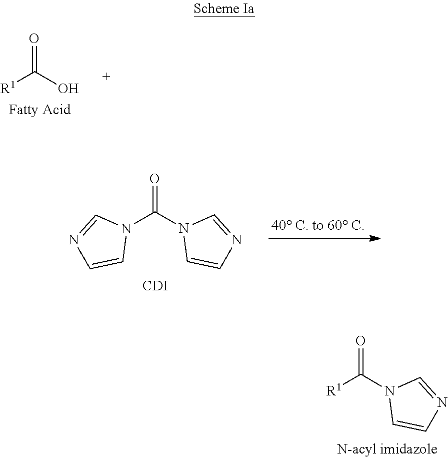

In certain aspects, one or more step(s) and/or reagent(s) described herein (e.g., for formation of the N-acyl imidazole) are excluded.

b) Formation of the Amide from the N-Acyl Imidazole and Primary Amine.

In some aspects, the amide is formed by a reaction between the N-acyl imidazole and a primary amine according to Scheme IIa. In certain aspects, the N-acyl imidazole and the primary amine have, e.g., are contacted at, a molar ratio equal to any one of, at least any one of, at most any one of, or between any two of 0.9:1, 0.91:0.99, 0.92:0.98, 0.93:0.97, 0.94:0.96, 0.95:0.95, 0.96:0.94, 0.97:0.93, 0.98:0.92, 0.99:0.91, and 1:0.9 (or any range derivable therein). In certain aspects, equal to any one of, at least any one of, at most any one of, or between any two of 0.85, 0.86, 0.88, 0.9, 0.92, 0.94, 0.95, 0.96, 0.98, 1, 1.02, 1.04, 1.06, 1.08, 1.1. 1.12, 1.14, 1.16, 1.18, and 1.2 moles (or any range derivable therein) of primary amine per mole fatty acid used to form the N-acyl imidazole (such as in Scheme Ia) is contacted with the N-acyl imidazole. In some aspects, at least a portion of the primary amine that is contacted with the N-acyl imidazole is in a melted form. In some aspects, the amine (e.g., in the melted form) is contacted with the N-acyl imidazole in an organic solvent such as toluene. In certain aspects, contacting of the N-acyl imidazole and the primary amine is performed at a temperature equal to any one of, at least any one of, at most any one of, or between any two of 40, 41, 42, 43, 44, 45, 46, 47, 48, 49, 50, 51, 52, 53, 54, 55, 56, 57, 58, 59, and 60° C. (or any range derivable therein). In some aspects, the contact/reaction temperature is not higher than 60° C., or higher than 65° C., or higher than 70° C.

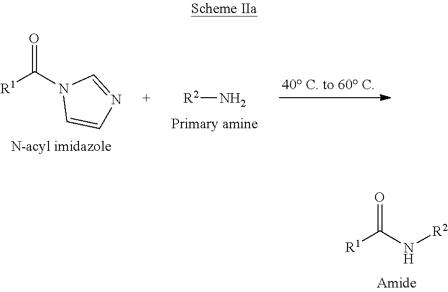

### 2. Formation of the Amide Via Acyl Chloride.

In some aspects, the fatty acid is contacted with an oxychloride to form an acyl chloride having a chemical formula of R¹—C(O)—Cl, and the acyl chloride is contacted with the primary amine to form the amide.

### a) Formation of the Acyl Chloride.

The acyl chloride can be formed by a reaction between the fatty acid and oxychloride according to Scheme lb. The oxychloride can be thionyl chloride, phosphoryl chloride, oxalyl chloride, or any combinations thereof. In some particular aspects, the oxychloride is oxalyl chloride. In certain aspects, the fatty acid and the oxychloride have, e.g., are contacted at, a molar ratio equal to any one of, at least any one of, at most any one of, or between any two of 1:0.8, 1:0.9, 1:1, 1:1.1, 1:1.2, 1:1.3, 1:1.4, 1:1.5, 1:1.6, 1:1.7, 1:1.8, 1:1.9, and 1:2 (or any range derivable therein). In some aspects, the fatty acid and the oxychloride are contacted in presence of dimethylformamide, and an organic solvent, such as benzene. In some aspects, the fatty acid and the oxychloride are contacted at a temperature equal to any one of, at least any one of, at most any one of, or between any two of 20, 25, 30, 35, 40, 45, 50, 55, 60, 65, 70 and 75° C. (or any range derivable therein). In certain aspects, the acyl chloride formed is concentrated in an organic solvent, such as toluene.

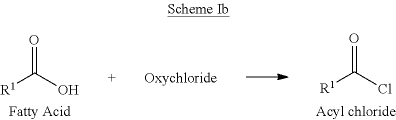

b) Formation of the Amide from the Acyl Chloride and Primary Amine.

In some aspects, the amide is formed by a reaction between the acyl chloride and a primary amine according to Scheme IIb. In some aspects, the acyl chloride and primary amine are contacted at a temperature equal to any one of, at least any one of, at most any one of, or between any two of 2, 4, 6, 8, 10, 12, 14, 16, 18, and 20° C. (or any range derivable therein). In some aspects, the acyl chloride and primary amine are contacted in presence of triethylamine, and an organic solvent, such as benzene. In some aspects, the amount of primary amine contacted with the acyl chloride, is equal to any one of, at least any one of, at most any one of, or between any two of 0.6, 0.62, 0.64, 0.65, 0.66, 0.68, 0.7, 0.72, 0.74, 0.75, 0.76, 0.78, 0.8, 0.82, 0.84, 0.85, 0.86, 0.88, 0.9, 0.92, 0.94, 0.95, 0.96, 0.98, 1, 1.02, 1.04, 1.06, 1.08, 1.1. 1.12, 1.14, 1.16, 1.18, and 1.2 moles (or any range derivable therein) of primary amine per mole of the fatty acid used to form the acyl chloride (such as in scheme Ib).

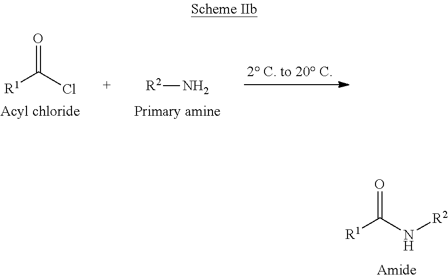

In certain aspects, the method further includes crystallization of the amide. The amide in an amidation-product mixture formed by the reaction of the primary amine and N-acyl imidazole (e.g., according to scheme IIa) or the primary amine and acyl chloride (e.g., according to scheme IIb), can be crystallized. The amidation-product mixture can contain i) the amide, ii) optionally unreacted reactants of the reaction of scheme Ia and IIa, or Ib and IIb; and iii) optionally side products and/or byproducts formed in the reaction of scheme Ia and IIa, or Ib and IIb. In certain aspects, the amidation-product mixture contains less than 4%, or less than 3%, or less than 2%, or less than 1%, by weight of the starting primary amine. Weight percent of the starting primary amine in the amidation-product mixture can be based on the total weight of the primary amine remaining after contact with the N-acyl imidazole (e.g., in scheme IIa) or acyl chloride (e.g., in scheme IIb), (e.g., added for amide formation). In a non-limiting example, if 100 gm of the primary amine is contacted with the N-acyl imidazole or acyl chloride, “an amidation-product mixture containing less than 4%, by weight of the starting primary amine,” refers to that the amount of unreacted primary amine in the amidation-product mixture is less than 4 gm. In some aspects, the crystallization of the amide includes adding isopropanol to the amidation-product mixture to form a crystallization mixture, and cooling the crystallization mixture to form amide crystals. In certain aspects, the crystallization mixture is formed by contacting isopropanol with the amidation-product mixture at a temperature equal to any one of, at least any one of, at most any one of, or between any two of 40, 40.1, 41, 42, 43, 44, 45, 46, 47, 48, 49, 50, 51, 52, 53, 54, 55, 56, 57, 58, 59, and 60° C. (or any range derivable therein).

In certain aspects, the cooling process of the crystallization mixture includes any one of, any combination of, or all of, steps (i) to (iv), where step (i) includes cooling the crystallization mixture equal to any one of, at least any one of, at most any one of, or between any two of 30, 31, 32, 33, 34, 35, 36, 37, 38, 39, and 40° C. (or any range derivable therein) to form a slurry; step (ii) includes maintaining the slurry at equal to any one of, at least any one of, at most any one of, or between any two of 30, 31, 32, 33, 34, 35, 36, 37, 38, 39, and 40° C. (or any range derivable therein) with continuous, periodic, or occasional stirring for at least 1 hour; step (iii) includes cooling the slurry equal to any one of, at least any one of, at most any one of, or between any two of 15, 16, 17, 18, 19, 20, 21, 22, 23, 24, and 25° C. (or any range derivable therein) with continuous, periodic, or occasional stirring, such as at 600 rpm or above; and step (iv) includes maintaining the slurry at equal to any one of, at least any one of, at most any one of, or between any two of 15, 16, 17, 18, 19, 20, 21, 22, 23, 24, and 25° C. (or any range derivable therein) with continuous, periodic, or occasional stirring for at least 0.5 hour. In some aspects, the slurry is cooled e.g., to 30° C. to 40° C. (step i) and/or to 15° C. to 25° C. (step iii) at a rate equal to any one of, at least any one of, at most any one of, or between any two of 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.1, 1.2, 1.3, 1.4, 1.5, 1.6, 1.7, 1.8, 1.9, and 2° C./min (or any range derivable therein).

A slurry formed by cooling the crystallization mixture or in part by cooling the crystallization mixture can contain the amide crystals (e.g., solid amide crystals). In certain aspects, the amide crystals are separated from the slurry (e.g., after step iv). In some particular aspects, the amide crystals are separated from the slurry via filtration. In some aspects, the process further includes washing the filtered amide crystals to form washed amide crystals, and drying the washed amide crystals. In certain aspects, the filtered amide crystals are washed with toluene and/or isopropanol. In certain aspects, the amide crystals (e.g., washed amide crystals) are dried at a temperature equal to any one of, at least any one of, at most any one of, or between any two of 40, 41, 42, 43, 44, 45, 46, 47, 48, 49, and 50° C. (or any range derivable therein). In some aspects, the amide crystals are dried at a temperature lower, e.g., at least 5° C. lower than the melting point of the amide. In certain aspects, during the amide crystallization and/or filtration of the slurry, the slurry is not cooled below 15° C., or below 10° C. or below 5° C. In certain aspects, the slurry is filtered at a pressure 0.4 barg or less, or 0.6 barg or less, or 0.8 barg or less, such as equal to any one of, at least any one of, at most any one of, or between any two of 0, 0.05, 0.1, 0.15, 0.2, 0.25, 0.3, 0.35, 0.4, 0.45, 0.5, 0.55, 0.6, 0.65, 0.7, 0.75, and 0.8 barg. In some aspects, the crystallization of the amide is performed in a reactor having a diameter D, where D is greater than the height H of the slurry in the reactor. In certain aspects, the reactor contains an impeller having a diameter D₁, where the ratio of D₁ and D is equal to any one of, at least any one of, at most any one of, or between any two of 0.35:1, 0.4:1, 0.45:1, 0.55:1, 0.6:1, and 0.65:1 (or any range derivable therein). The slurry formed during crystallization of the amide can be stirred with the impeller. Amide with high purity can be obtained through crystallization, and purity of the compound of Formula I produced can be increased by using the crystallized amides for producing the compound of Formula I (e.g., via the secondary amine).

In certain other aspects, the crystallization of the amide includes any one of, any combination of, or all of, steps i′) to iii′), where step i′) includes adding water and an acid to the amidation-product mixture to form a mixture having a pH of 5.5 to 7.5 or any value or range derivable therein or there between, such as 5.5, 6, 6.5, 7, or 7.5, wherein the amide precipitates from the mixture, step ii′) includes separating the amide precipitate, and iii′) includes crystallizing of the separated amide precipitate in methanol. In some aspects, the acid in step i′) is sulfuric acid. In some aspects, the amide precipitate is separated in step ii′) by filtration.

In certain aspects, one or more step(s) and/or reagent(s) described herein (e.g., for formation of the amide from the primary amine and N-acyl imidazole or the primary amine and acyl chloride) are excluded. In some particular aspects, the amide is formed from a fatty acid and the primary amine, via N-acyl imidazole (e.g., scheme Ia and IIa).

B. Formation of the Secondary Amine from the Amide.

A secondary amine can be formed from the amide according to Scheme III. The amide can be reduced by a reducing agent to form the secondary amine. In certain aspects, the reducing agent is a hydride. In some particular aspects, the hydride is lithium aluminum hydride. In certain aspects, the amide and the reducing agent have, e.g., are contacted, at a molar ratio equal to any one of, at least any one of, at most any one of, or between any two of 1:1, 1:1.2, 1:1.4, 1:1.6, 1:1.8, 1:2, 1:2.2, 1:2.4, 1:2.6, 1:2.8, and 1:3 (or any range derivable therein). In certain aspects, an amide solution containing the amide is contacted with a reducing agent solution containing the reducing agent. The amide solution and the reducing agent solution can independently further contain one or more organic solvent(s). In some aspects, the reducing agent solution contains the reduction agent, and tetrahydrofuran (THF), and/or 2-methyl THF. In some particular aspects, the reducing agent solution contains the reduction agent and THF. In some aspects, the amide solution contains the amide and toluene. In some aspects, the amide solution is formed by contacting the amide crystals (e.g., formed as described herein) with one or more organic solvents, such as toluene. In some aspects, contacting of the amide and the reducing agent is performed at a temperature equal to any one of, at least any one of, at most any one of, or between any two of 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, and 75° C. (or any range derivable therein). The reduction of the amide can be quenched using sodium sulfate. In certain aspects, a reduction-product mixture formed by the reduction of the amide is contacted with sodium sulfate to quench the reduction of the amide. The reduction-product mixture can contain i) the secondary amine; ii) optionally unreacted reactants such as the amide and/or the reducing agent, and iii) optionally side products and/or byproducts formed during the reduction of the amide. In certain aspects, the reduction-product mixture contains less than 4%, or less than 3%, or less than 2%, or less than 1%, by weight of the starting amide. Weight of the starting amide can be the total weight of the amide that is contacted with the reducing agent (e.g., added for reduction). In a non-limiting example, if 100 gm of the amide is contacted with the reducing agent, “a reduction-product mixture containing less than 4%, by weight of the starting primary amide,” refers to that the amount of unreacted amide in the reduction-product mixture is less than 4 gm. In some aspects, the quenching process includes contacting the reduction-product mixture with a slurry or solution comprising sodium sulfate at a temperature equal to any one of, at least any one of, at most any one of, or between any two of 35, 36, 37, 38, 39, 40, 41, 42, 43, 44, and 45° C. (or any range derivable therein) to form a quenched reduction-product mixture and residual sodium sulfate. In certain aspects, the residual sodium sulfate is separated from the quenched reduction-product mixture. In certain aspects, equal to any one of, at least any one of, at most any one of, or between any two of 0.5, 0.6, 0.7, 0.8, 0.9, 1, 1.1, 1.2, 1.3, 1.4, 1.5, 1.6, 1.7, 1.8, 1.9, and 2 moles (or any range derivable therein) of sodium sulfate per mole of amide reduced (starting amide) is contacted with the reduction-product mixture. In certain aspects, the slurry or solution containing sodium sulfate further contains an organic solvent. In certain aspects, the slurry containing sodium sulfate further contains THF and/or toluene. In some particular aspects, the slurry containing sodium sulfate contains THF. In certain aspects, residual sodium sulfate (e.g., at least a portion of the residual sodium sulfate) is separated from the quenched reduction-product mixture by filtration, wherein a separated reduction-product mixture containing the secondary amine is formed as a filtrate.

In certain aspects, the method further includes forming a crystallized salt of the secondary amine. In certain aspects, the crystallized salt of the secondary amine is formed by contacting the secondary amine with an acid to form a salt-forming solution containing a salt of the secondary amine, and cooling the salt-forming solution to form the crystallized salt of the secondary amine. In certain aspects, the secondary amine is contacted with isopropanol and the acid at a temperature of equal to any one of, at least any one of, at most any one of, or between any two of 50, 51, 52, 53, 54, 55, 56, 57, 58, 59, and 60° C. (or any range derivable therein) to form the salt-forming solution. In certain aspects, the secondary amine in the separated reduction-product mixture (e.g., formed by separation of at least a portion of the residual sodium sulfate from the quenched reduction-product mixture) is contacted with isopropanol and the acid to form the salt-forming solution. In certain aspects, the cooling of the salt-forming solution includes, any one of, any combination of, or all of, steps (i′) to (iii′), where, step (i′) includes cooling the salt-forming solution at a temperature equal to any one of, at least any one of, at most any one of, or between any two of 35, 36, 37, 38, 39, 40, 41, 42, 43, 44, and 45° C. (or any range derivable therein); step (ii′) includes maintaining the salt-forming solution at a temperature equal to any one of, at least any one of, at most any one of, or between any two of 35, 36, 37, 38, 39, 40, 41, 42, 43, 44, and 45° C. (or any range derivable therein) for at least 1 hour; and step (iii′) includes cooling the salt-forming solution to a temperature equal to any one of, at least any one of, at most any one of, or between any two of 15, 16, 17, 18, 19, 20, 21, 22, 23, 24, and 25° C. (or any range derivable therein). The cooled salt-forming solution can contain crystallized salt of the secondary amine. In certain aspects, the crystallized salt of the secondary amine is separated from the salt-forming solution (e.g., after step iii′). In some particular aspects, the crystallized salt of the secondary amine is separated from the salt-forming solution by filtration, wherein the crystallized salt (e.g., solid crystallized salt) is obtained as a filtered residue. In certain aspects, after separation from the salt-forming solution the crystallized salt (e.g., the crystallized salt in the filtered residue) is washed and dried. In certain aspects, the crystallized salt of the secondary amine is washed with a solution containing toluene and/or isopropanol. In certain aspects, the solution (e.g., used for washing) contains toluene and isopropanol at a volume % ratio equal to any one of, at least any one of, at most any one of, or between any two of 0.9:1, 0.91:0.99, 0.92:0.98, 0.93:0.97, 0.94:0.96, 0.95:0.95, 0.96:0.94, 0.97:0.93, 0.98:0.92, 0.99:0.91, and 1:0.9 (or any range derivable therein). The washed crystallized salts of the secondary amine (e.g., formed by washing with the solution containing toluene and/or isopropanol) can be dried i) at a pressure equal to any one of, at least any one of, at most any one of, or between any two of 0, 0.05, 0.1, 0.15 and 0.2 bar (or any range derivable therein); and/or ii) a temperature equal to any one of, at least any one of, at most any one of, or between any two of 40, 41, 42, 43, 44, 45, 46, 47, 48, 49, and 50° C. (or any range derivable therein). In certain aspects, the acid (e.g., the acid contacted with the secondary amine to form the salt-forming solution) is hydrochloric acid, hydrobromic acid, hydroiodic acid, sulfuric acid, acetic acid, methanesulfonic acid, toluenesulfonic acid, (1R)-(-)-10-camphorsulfonic acid, 1,2-ethanedisulfonic acid, oxalic acid, dibenzoyl-L-tartaric acid, phosphoric acid, L-tartaric acid, maleate, fumaric acid, succinic acid, and/or malonic acid. In some particular aspects, the acid is succinic acid. In some particular aspects, the acid is L-tartaric acid. In certain aspects, the salt of the secondary amine contains a cation having the formula of Formula II, and an anion selected from chloride, bromide, iodide, sulfate, acetate, mesylate, tosylate, (1R)-(-)-10-camphorsulfonate, 1,2-ethanedisulfonate, oxalate, dibenzoyl-L-tartarate, phosphate, L-tartarate, maleate, fumarate, succinate, and malonate. In certain aspects, the cation has the formula of Formula IIa. In certain aspects, the anion is succinate. In certain aspects, the anion is L-tartarate. In certain aspects, the cation has the formula of Formula IIa and the anion is succinate. In certain aspects, the cation has the formula of Formula IIa and the anion is L-tartarate.

In certain aspects, the secondary amine formed from the amide, such as the secondary amine in the separated reduction-product mixture is precipitated, and the precipitated secondary amine is crystallized in methanol.

In certain aspects, one or more step(s) and/or reagent(s) described herein (e.g., for formation of the secondary amine and crystallized salts of the secondary amine) are excluded.

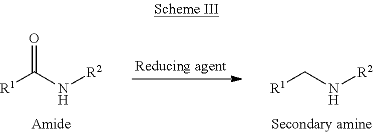

C. Formation of the Compound of Formula I from the Secondary Amine

The secondary amine and the polyolefin-glycol compound can be coupled to form the compound of Formula I. In certain aspects, the crystals of the secondary amine are dissolved in an organic solvent and can be used for formation of the compound of Formula I. In certain aspects, the secondary amine used for formation of the compound of Formula I, is formed from the crystallized salts of the secondary amine. Secondary amine with high purity can be obtained through forming salts of the secondary amine (e.g., as described herein), crystallization of the salts of the secondary amine (e.g., as described herein), and reforming the secondary amine from the crystallized salts. Purity of the compound of Formula I produced can be increased by using the reformed secondary amine for producing the compound of Formula I. In certain aspects, the secondary amine is reformed from the crystallized salt of the secondary amine by contacting the crystallized salts (e.g., dried, crystallized salts as described herein) with a base. In certain aspects, the base is NaOH and/or KOH. In certain aspects, the base is KOH. In certain aspects, the secondary amine is reformed from the dried, crystallized salts of the secondary amine by contacting the dried, crystallized salts with an organic solvent to form a salt solution; washing the salt solution with the base and water to form a washed organic solution containing the secondary amine; and distilling the washed organic solution to form a distilled organic solution containing the secondary amine. In certain aspects, the salt solution is washed with the base and/or water more than once. In certain aspects, the dried, crystallized salts are contacted with toluene to form the salt solution. In certain aspects, the washed organic solution contains less than 200 parts per million by weight (ppmw), or less than 150 ppmw, or less than 100 ppmw of the acid (e.g., acid used for formation of the salts of the secondary amine). The salt solution can be washed with the base and/or water one or more times until the washed organic solution formed by washing contains less than 200 ppmw, or less than 150 ppmw, or less than 100 ppmw of the acid. In certain aspects, the washed organic solution is distilled i) at a pressure equal to any one of, at least any one of, at most any one of, or between any two of 0, 0.05, 0.1, 0.15, 0.2, 0.25, and 0.3 bar (or any range derivable therein); and/or ii) a temperature at or below 70° C., below 65° C., or below 60° C., to form the distilled organic solution. In some aspects, the distilled organic solution contains less than 0.5 wt. %, or less than 0.1 wt. %, or less than 0.05 wt. %, of water. In certain aspects, the secondary amine in the distilled organic solution is used, e.g., contacted with the activated polyolefin-glycol compound to form the compound of Formula I. In certain aspects, the distilled organic solution is contacted with the activated polyolefin-glycol compound. The activated polyolefin-glycol compound and the secondary amine can have, e.g., can be contacted at, a molar ratio equal to any one of, at least any one of, at most any one of, or between any two of 1:1.2, 1:1.25, 1:1.3, 1:1.35, 1:1.4, 1:1.45, and 1:1.5 (or any range derivable therein).

In certain aspects, the polyolefin-glycol compound is a polyolefin glycol acid having a chemical formula of HOOC—L—(O(CH₂)ₙ)ₘ—OR³, and the secondary amine and the polyolefin glycol acid are coupled according to Scheme IVa to form the compound of Formula I. In certain aspects, the polyolefin glycol acid is a polyethylene glycol acid, having a chemical formula of HOOC—CH₂—(O(CH₂)₂)ₘ—OR³. In certain aspects, the polyolefin glycol acid is contacted with an organic base and a coupling agent to form a coupling solution containing an activated polyolefin-glycol compound. In certain aspects, the organic base is a tertiary amine. In certain aspects, the tertiary amine is diisopropylethylamine. In certain aspects, the coupling agent is 1-propanephosphonic acid cyclic anhydride. In certain aspects, the coupling solution is formed by contacting the polyolefin glycol acid and the organic base at a molar ratio equal to any one of, at least any one of, at most any one of, or between any two of 1:3.5, 1:3.6, 1:3.7, 1:3.8, 1:3.9, 1:4, 1:4.1, 1:4.2, 1:4.3, 1:4.4, and 1:4.5 (or any range derivable therein). In certain aspects, the coupling solution is formed by contacting the polyolefin glycol acid and the coupling agent at a molar ratio equal to any one of, at least any one of, at most any one of, or between any two of 1:1.8, 1:1.85, 1:1.9, 1:1.95, 1:2, and 1:2.2 (or any range derivable therein). In certain aspects, the coupling solution is formed by contacting the polyolefin glycol acid with an organic solvent to form a polyolefin glycol solution, distilling the polyolefin glycol solution to form a distilled polyolefin glycol solution, and contacting the distilled polyolefin glycol solution with the organic base and coupling agent to form the coupling solution containing the activated polyolefin-glycol compound. In certain aspects, the polyolefin glycol solution is distilled i) at a pressure equal to any one of, at least any one of, at most any one of, or between any two of 0, 0.05, 0.1, 0.15, and 0.2 bar (or any range derivable therein); and/or ii) a temperature at or below 70° C., below 65° C., or below 60° C., to form the distilled polyolefin glycol solution. In certain aspects, the distilled polyolefin glycol solution contains less than 0.5 wt. %, or less than 0.1 wt. %, or less than 0.05 wt. %, of water. In certain aspects, the polyolefin glycol acid is contacted with toluene to form the polyolefin glycol solution. In certain aspects, the coupling solution is colorless. In certain aspects, the coupling solution is contacted with the secondary amine to form the compound of Formula I. In certain aspects, the coupling solution is contacted with a solution formed by dissolving the secondary amine crystals. In certain aspects, the coupling solution is contacted with the reformed secondary amine (e.g., reformed from the crystallized secondary amine salts) to form the compound of Formula I. In certain aspects, the coupling solution is contacted with the distilled organic solution containing the reformed secondary amine to form the compound of Formula I. In certain aspects, the compound of Formula I is formed (e.g., from using the polyolefin glycol acid and the secondary amine) at a temperature equal to any one of, at least any one of, at most any one of, or between any two of 20, 21, 22, 23, 24, 25, 26, 27, 28, 29, 30, 31, 32, 33, 34, 35, 36, 37, 38, 39, 40, 41, 42, 43, 44, and 45° C. (or any range derivable therein). In some instances, an additive is added to the coupling solution, to the secondary amine and/or the reformed secondary amine, and/or during or after the coupling solution contacts the secondary amine and/or the reformed secondary amine to boost the reaction efficiency. In some instances, the additive is pyridine hydrobromide. In certain aspects, the compound of Formula I is formed at a yield equal to any one of, at least any one of, at most any one of, or between any two of 80, 81, 82, 83, 84, 85, 86, 87, 88, 89, 90, 91, 92, 93, 94, 95, 96, 97, 98, and 99% (or any range derivable therein). In certain aspects, the compound of Formula I is formed at an in situ yield of 89 to 95%, or any range derivable therein.

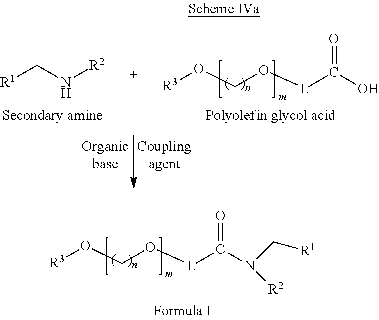

In certain aspects, the polyolefin-glycol compound is a N-hydroxylsuccinimide (NHS) functionalized polyolefin glycol, and has a chemical formula of NHS—O(O)C—L—(O(CH₂)ₙ)ₘ—OR³. The NHS functionalized polyolefin glycol and the secondary amine can be coupled according to Scheme IVb to form the compound of Formula I. In certain aspects, the NHS functionalized polyolefin glycol, is a NHS functionalized polyethylene glycol having a chemical formula of NHS—O(O)C-CH₂—(O(CH₂)₂)ₘ—OR³. In some aspects, the NHS functionalized polyolefin glycol is contacted with the secondary amine at a molar ratio equal to any one of, at least any one of, at most any one of, or between any two of 0.6:1, 0.7:1, 0.8:1, 0.9:1, 1:1, 1.1:1, and 1.2:1 (or any range derivable therein). In certain aspects, the NHS functionalized polyolefin glycol is contacted with the secondary amine in presence of a tertiary amine. In some particular aspects, the tertiary amine is triethylamine. In some aspects, the NHS functionalized polyolefin glycol is contacted with the secondary amine at a temperature equal to any one of, at least any one of, at most any one of, or between any two of 20, 21, 22, 23, 24, 25, 26, 27, 28, 29, 30, 31, 32, 33, 34, 35, 36, 37, 38, 39, 40, 41, 42, 43, 44, and 45° C. (or any range derivable therein). In certain aspects, the NHS functionalized polyolefin glycol is contacted with a solution formed by dissolving the secondary amine crystals. In certain aspects, the NHS functionalized polyolefin glycol is contacted with the reformed secondary amine (e.g., reformed from the crystallized secondary amine salts) to form the compound of Formula I. In certain aspects, the NHS functionalized polyolefin glycol is contacted with the distilled organic solution containing the reformed secondary amine to form the compound of Formula I. In some instances, an additive is present in a mixture of the NHS functionalized polyolefin glycol and secondary amine and/or the reformed secondary amine to boost the reaction efficiency. In some instances, the additive is pyridine hydrobromide. In certain aspects, the compound of Formula I is formed at a yield equal to any one of, at least any one of, at most any one of, or between any two of 80, 81, 82, 83, 84, 85, 86, 87, 88, 89, 90, 91, 92, 93, 94, 95, 96, 97, 98, and 99% (or any range derivable therein). In certain aspects, the compound of Formula I is formed at an in situ yield of 89 to 95%, or any range derivable therein.

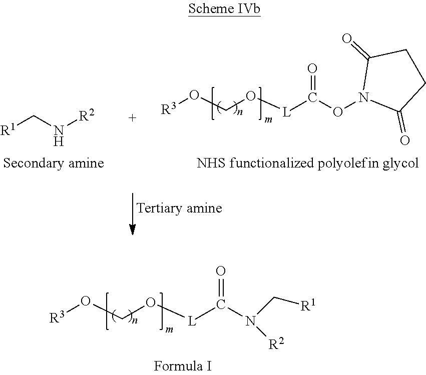

In certain aspects, the reaction of Scheme IVa and/or IVb is quenched using potassium carbonate and sodium chloride. In certain aspects, the quenching process (e.g., quenching of the reaction of Scheme IVa and/or IVb) includes contacting an aqueous quench solution containing potassium carbonate and sodium chloride, with a coupling-product mixture to form a biphasic product mixture containing i) an organic phase containing the compound of Formula I, and ii) an aqueous phase; separating the organic phase and the aqueous phase of the biphasic product mixture; and distilling the organic phase to form a product solution containing the compound of Formula I.

The coupling-product mixture can be formed from the reaction of Scheme IVa and/or IVb. The coupling-product mixture can contain i) the compound of Formula I, ii) optionally unreacted reactants from the reaction of Scheme IVa and/or IVb, and iii) optionally side products and/or byproducts formed in the reaction of Scheme IVa and/or IVb. In certain aspects, the coupling-product mixture contains less than 15 wt. %, or less than 10 wt. %, or less than 5 wt. %, or less than 4 wt. %, or less than 3 wt. %, or less than 2 wt. %, or less than 1 wt. % of the starting secondary amine, or any number there between. Weight of the starting secondary amine can be the total weight of secondary amine remaining after being contacted with the polyolefin-glycol compound (e.g., added for formation of the compound of Formula I). In a non-limiting example, if 100 gm of the secondary amine is contacted with the polyolefin-glycol compound, “a coupling-product mixture containing less than 4%, by weight of the starting secondary amine,” refers to that the amount of unreacted secondary amine in the coupling-product mixture is less than 4 gm. In certain aspects, the product solution (e.g., obtained by distilling the organic phase) contains less than 0.5 wt. %, or less than 0.12 wt. %, or less than 0.1 wt. %, or less than 0.05 wt. %, of water. In certain aspects, the organic phase is distilled i) at a pressure equal to any one of, at least any one of, at most any one of, or between any two of 0, 0.05, 0.1, 0.15, and 0.2 bar (or any range derivable therein); and/or ii) a temperature at or below 70° C., below 65° C., or below 60° C., to form the product solution.

In certain aspects, the method further includes purifying the compound of Formula I. In some aspects, the compound of Formula I is purified by silica gel chromatography or polymer resin chromatography. In certain aspects, compound of Formula I in the product solution (e.g., formed through quenching of the reaction between the secondary amine and the polyolefin-glycol compound) is purified by silica gel chromatography or polymer resin chromatography to form the purified compound of Formula I.

In certain aspects, the method further includes precipitating the purified compound of Formula I. In certain aspects, the method does not include precipitating the purified compound of Formula I. In certain aspects, the precipitation process of the purified compound of Formula I includes forming an ethanol solution of the purified compound of Formula I and contacting isopropanol with the ethanol solution to form an isopropanol and ethanol mixture, wherein the compound of Formula I precipitates from the isopropanol and ethanol mixture. In certain aspects, the compound of Formula I is purified by silica gel chromatography, and an ethanol solution of the purified compound is formed by eluting the silica gel chromatography column (e.g., eluting a silica gel chromatography column loaded with compound of Formula I) with ethanol. In certain aspects, the ethanol solution (e.g., obtained from eluting the silica gel chromatography column) and/or isopropanol and ethanol mixture containing compound of Formula I is distilled at a pressure of 0 to 0.2 bar and/or a temperature below 40° C.

In certain aspects, the isopropanol and ethanol mixture contains isopropanol and is ethanol at a weight ratio equal to any one of, at least any one of, at most any one of, or between any two of 2.5:1, 2.6:1, 2.7:1, 2.8:1, 2.9:1, 3:1, 3.2:1, 3.3:1, 3.4:1, and 3.5:1 (or any range derivable therein). In certain aspects, the precipitate of the compound of Formula I is separated from the isopropanol and ethanol mixture. In some particular aspects, the precipitate of the compound of Formula I is separated from the isopropanol and ethanol mixture by filtration. In certain aspects, the precipitation method of the compound of Formula I, and/or separation method of the compound of Formula I do not include solvent lyophilization. Formation of the compound of Formula I in solid form can help in purification, storage, and transportation of the compound. Generally solid polymer conjugated lipids are formed by solvent lyophilization, however lyophilization process can be energy and time demanding. Formation of the compound of Formula I in solid form (e.g., precipitate) without use of lyophilization can make the solid formation step more cost and time effective. In some aspects, the precipitate of the compound of Formula I is a mixed solid phase compound containing crystalline solids and amorphous solid.

In some other aspects, the method includes isolating the compound of Formula I in a solid form, from a reaction mixture formed by contacting the secondary amine and the polyolefin glycol compound. In some instances, isolating in a sold form includes concentrating the reaction mixture, a purified compound of Formula I, or a partially purified compound of Formula I. In some instances, concentrating includes removing solvent via lyophilization, heating, reduced pressure, gas flow, filtration, precipitation, and/or centrifugation, etc. In some instances, concentrating excludes removing solvent via lyophilization, heating, reduced pressure, gas flow, filtration, precipitation, and/or centrifugation. In some instances, concentrating excludes removing solvent via lyophilization, The process can include, any one of, any combination of, or all of steps i″) to v″). Step i″) includes washing the reaction mixture after a desired amount of time with water to form an aqueous phase containing the compound of Formula I. Step ii″) includes extracting the aqueous phase with an organic solvent, such as DCM, to obtain an organic solution containing the compound of Formula I. Step iii″) includes washing, drying, concentrating, and/or cooling the organic solution to precipitate at least a portion of residual starting secondary amine from the organic solution. Step iv″) includes filtering the organic solution to separate the precipitated secondary amine, and adding triethylamine and acetic anhydride to the filtrate. Step v″) includes concentrating the filtrate, to obtain the compound of Formula I in solid form. In some aspects, the organic solution in step iii″) is washed with a salt solution such as brine. In some aspects, the organic solution in step iii″) is dried over sodium sulfate. In some aspects, the organic solution in step iii″) is cooled to 0, −5, −10, −15, −20, or −25° C. (or any ranges or values derivable therein). In certain aspects, compound of Formula I obtained through a solvent concentration process (e.g., as mentioned in this paragraph), is further purified, such as by silica gel chromatography. In certain aspects, isolation of the compound of Formula I in a solid form, through solvent concentration (e.g. as mentioned in this paragraph) is not performed.

In certain aspects, one or more step(s) and/or reagent(s) described herein (e.g., for formation of the compound of Formula I from the secondary amine and/or precipitation of compound of Formula I) are excluded.

### III. Salts of the Polymer Conjugated Lipids and Intermediates Thereof

Certain aspects are directed a salt (e.g., salts of the secondary amine) containing a cation having the formula of Formula II

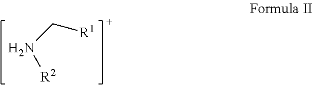

wherein R¹ and R² are as described above, and

an anion selected from chloride, bromide, iodide, sulfate, acetate, mesylate, tosylate, (1R)-(-)-10-camphorsulfonate, 1,2-ethanedisulfonate, oxalate, dibenzoyl-L-tartarate, phosphate, L-tartarate, maleate, fumarate, succinate, and malonate. In certain aspects, the anion is succinate. In certain aspects, the anion is L-tartarate. In certain aspects, the cation of the salt has the formula of Formula IIa

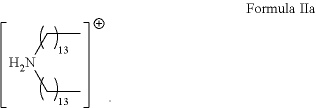

In certain aspects, the salt is in a crystallized form. In certain aspects, the salt is a stoichiometric salt. In certain aspects, the salt is a non-stoichiometric salt. In certain aspects, the salt is a non-stoichiometric salt containing the cation of Formula II and succinate. In certain aspects, the salt is a non-stoichiometric salt containing the cation of Formula II and L-tartarate. In some particular aspects, the salt is a non-stoichiometric salt containing the cation of Formula IIa and succinate. In some particular aspects, the salt is a non-stoichiometric salt containing the cation of Formula IIa and L-tartarate. The salts (e.g., of the secondary amine) can have a melting point higher than the corresponding secondary amine. Higher melting point can enable easier storage and shipping of the salt. The salts can be prepared by methods described herein. In certain aspects, one or more salts described herein are excluded. In certain aspects, one or more steps, and/or reagents for making the salts described herein are excluded.

### IV. Use of Compounds of Formula I, Intermediates Thereof, Salts Thereof and Salts of Intermediates; and Compositions Containing the Compound of Formula I, Intermediates Thereof, Salts Thereof, and Salts of Intermediates.

Certain aspects are directed to a use of a polymer conjugated lipid described herein, an intermediate for the production thereof (e.g., the N-acyl imidazole, amide, and/or secondary amine), a pharmaceutically acceptable salt of the lipid, and/or pharmaceutically acceptable salt of the intermediate (e.g., of the secondary amine). Certain aspects are directed to a composition containing a polymer conjugated lipid described herein, an intermediate for the production thereof (e.g., the N-acyl imidazole, amide, and/or secondary amine), a pharmaceutically acceptable salt of the lipid, and/or pharmaceutically acceptable salt of the intermediate (e.g., of the secondary amine). The polymer conjugated lipid described herein, an intermediate thereof, salt thereof, and salt of the intermediate (e.g., of the secondary amine) can be synthesized using a method described herein.

The polymer conjugated lipids and/or pharmaceutically acceptable salts thereof, optionally in combination with other lipids, can be used for intracellular delivery of a therapeutic agent. In certain aspects, the therapeutic agent can be a nucleic acid. In certain aspects, the nucleic acid can be messenger RNA (mRNA), nucleoside-modified mRNA, antisense oligonucleotides, ribozymes, DNAzymes, plasmids, immune stimulating nucleic acids, antagomirs, anti-miRs, miRNA mimics, supermirs, and/or aptamers. In some particular aspects, the nucleic acid can be antisense, plasmid DNA, and/or nucleoside-modified mRNA.

Certain aspects, are directed to a pharmaceutical composition containing a polymer conjugated lipid described herein, an intermediate for the production thereof (e.g., the acyl chloride, ester alcohol, and/or ester aldehyde), a pharmaceutically acceptable salt of the lipid, and/or pharmaceutically acceptable salt of the intermediate (e.g., of the secondary amine); and a therapeutic agent. In certain aspects, the polymer conjugated lipid, the intermediate and/or the pharmaceutically acceptable salt thereof can be in a lipid nanoparticle form. The lipid nanoparticle can have at least one dimension on the order of nanometers (e.g., 1-1,000 nm), and can include one or more lipids. In some aspects, the lipid nanoparticle can further include or exclude one or more excipient selected from neutral lipids, charged lipids, steroids, and polymer conjugated lipids. In some aspects, the therapeutic agent such as the nucleoside-modified RNA can be encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells, e.g., an adverse immune response. In certain aspects, the lipid nanoparticles have an average diameter of from about, equal to any one of, at least any one of, at most any one of, or between any two of 30 nm to about 150 nm, about 40 nm to about 150 nm, about 50 nm to about 150 nm, about 60 nm to about 130 nm, about 70 nm to about 110 nm, about 70 nm to about 100 nm, about 80 nm to about 100 nm, about 90 nm to about 100 nm, about 70 to about 90 nm, about 80 nm to about 90 nm, about 70 nm to about 80 nm, or equal to any one of, at least any one of, at most any one of, or between any two of about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some aspects the lipid nanoparticles are substantially non-toxic. In certain aspects, the nucleoside-modified RNA, when present in the lipid nanoparticles, is resistant in aqueous solution to degradation by a nuclease.

Administration of the compositions described herein can be carried out via any of the accepted modes of administration of agents for serving similar utilities. Pharmaceutical compositions may be formulated into preparations in solid, semi-solid, liquid or gaseous forms, such as tablets, capsules, powders, granules, ointments, solutions, suspensions, suppositories, injections, inhalants, gels, microspheres, and aerosols. Typical routes of administering such pharmaceutical compositions include, without limitation, oral, topical, transdermal, inhalation, parenteral, sublingual, buccal, rectal, vaginal, and intranasal. The term parenteral as used herein includes subcutaneous injections, intravenous, intramuscular, intradermal, intrasternal injection, or infusion techniques. Pharmaceutical compositions described herein are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound in aerosol form may hold a plurality of dosage units. The composition to be administered will, in any event, contain a therapeutically effective amount of a compound within the scope of this disclosure, or a pharmaceutically acceptable salt thereof, for treatment of a disease or condition of interest in accordance with the teachings described herein.

A pharmaceutical composition within the scope of this disclosure may be in the form of a solid or liquid. In one aspect, the carrier(s) are particulate, so that the compositions are, for example, in tablet or powder form. The carrier(s) may be liquid, with the compositions being, for example, an oral syrup, injectable liquid, or an aerosol, which is useful in, for example, inhalator administration. When intended for oral administration, the pharmaceutical composition is preferably in either solid or liquid form, where semi-solid, semi-liquid, suspension, and gel forms are included within the forms considered herein as either solid or liquid. As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present or exclude: binders such as carboxymethylcellulose, ethyl cellulose, microcrystalline cellulose, gum tragacanth, or gelatin; excipients such as starch, lactose, or dextrins; disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate, or orange flavoring; and a coloring agent. When the pharmaceutical composition is in the form of a capsule, for example, a gelatin capsule, it may contain, in addition to materials of the above type, a liquid carrier such as polyethylene glycol or oil. The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant, and flavor enhancer. In a composition intended to be administered by injection, one or more of a surfactant, preservative, wetting agent, dispersing agent, suspending agent, buffer, stabilizer, and isotonic agent may be included or exclude.

A liquid pharmaceutical composition, whether they be solutions, suspensions or other like form, may include or exclude one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates, or phosphates; and agents for the adjustment of tonicity such as sodium chloride or dextrose; agents to act as cryoprotectants such as sucrose or trehalose. The parenteral preparation can be enclosed in ampoules, disposable syringes, or multiple dose vials made of glass or plastic. Physiological saline is a preferred adjuvant. An injectable pharmaceutical composition is preferably sterile.

A liquid pharmaceutical composition intended for either parenteral or oral administration can contain an amount of a compound such that a suitable dosage will be obtained.

The pharmaceutical compositions may be prepared by methodology well known in the pharmaceutical art. For example, a pharmaceutical composition intended to be administered by injection can be prepared by combining the lipid nanoparticles with sterile, distilled water or other carrier so as to form a solution. A surfactant may be added to facilitate the formation of a homogeneous solution or suspension. Surfactants are compounds that non-covalently interact with a compound consistent with the teachings herein so as to facilitate dissolution or homogeneous suspension of the compound in the aqueous delivery system.

The compositions within the scope of the disclosure, or their pharmaceutically acceptable salts, are administered in a therapeutically effective amount, which will vary depending upon a variety of factors including the activity of the specific therapeutic agent employed; the metabolic stability and length of action of the therapeutic agent; the age, body weight, general health, gender, and diet of the patient; the mode and time of administration; the rate of excretion; the drug combination; the severity of the particular disorder or condition; and the subject undergoing therapy.

### EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

### Example 1

Producing amide (C-1) from myristic acid and 1-tetradecylamine.

An amide (C-1) was formed according to the Scheme Ela.

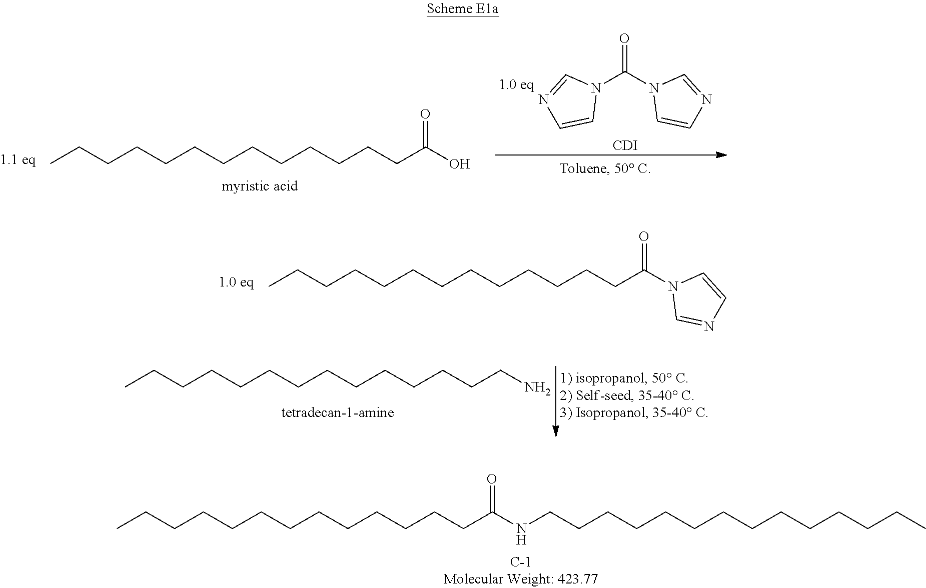

Material used for forming the amide (C-i) is listed in Table 1. Equiv. and eq are used for equivalent.

TABLE 1
Material used

| | | | | Molar | |
|---|---|---|---|---|---|
| | MW or | Weight or | | Ratio or | |
| Materials | Density | Volume | mmol | ml/g | Comments |

Step 1 Reaction and Crystallization Tank

| | | | | | | | | |
|---|---|---|---|---|---|---|---|---|
| Toluene (Solvent for | 92.14, 0.871 | 100 | mL | 945 | mmol | 2 | mL/g | anhydrous (<0.1% |
| CDI) | g/mL | | | | | | | water) |
| 1,1′-Carbonyldiimidazole | 162.15 | 37.21 | g | 222.6 | mmol | 1 | equiv. | 97wt %, potency can |
| (CDI) | | | | | | | | vary, typical bottles |
| | | | | | | | | from Aldrich are >90 |
| | | | | | | | | wt % potency. |
| Toluene (Chase for | 92.14, 0.871 | 25 | ml | 236 | mmol | 0.5 | ml/g | |
| Reactor #2 to Reactor #1 | g/mL | | | | | | | |
| transfer) | | | | | | | | |
| 1-Tetradecylamine | 213.4 | 50 | g | 222.6 | mmol | 50 | g | Typically >95 wt %, |
| Limiting Reagent (LR) | | | | | | | | charges are adjusted |
| | | | | | | | | by the potency of LR |
| | | | | | | | | assuming 95 wt % |
| | | | | | | | | Low melting point |
| | | | | | | | | solid (37-40° C.) |
| Imidazole (By-product) | 68.08 | 30.31 | g | 445.2 | mmol | 2 | equiv. | Theoretical amount |
| Carbon Dioxide (By- | 44.01 | 9.8 | g | 222.6 | mmol | 1 | equiv. | Theoretical amount |
| product) | | | | | | | | |
| Isopropanol (First anti- | 60.10, 0.786 | 250 | ml | 3270 | mmol | 5 | ml/g | anhydrous (<0.1% |
| solvent charge) | g/ml | | | | | | | water) |
| Isopropanol (Second | 60.10, 0.786 | 250 | ml | 3270 | mmol | 5 | ml/g | anhydrous (<0.1% |
| anti-solvent charge) | g/ml | | | | | | | water) |
| Isopropanol (First cake | 60.10, 0.786 | 200 | ml | 2616 | mmol | 4 | ml/g | anhydrous (<0.1% |
| wash) | g/ml | | | | | | | water) |
| Toluene (First cake | 92.14, 0.871 | 100 | mL | 945 | mmol | 2 | mL/g | anhydrous (<0.1% |
| wash) | g/mL | | | | | | | water) |
| Toluene (Second cake | 92.14, 0.871 | 300 | mL | 2836 | mmol | 6 | mL/g | anhydrous (<0.1% |
| wash) | g/mL | | | | | | | water) |
| Amide (C-1) | 423.8 | 96.36 | g | 222.6 | mmol | 1 | equiv. | Theoretical potency- |
| | | | | | | | | adjusted yield. Low |
| | | | | | | | | melting point solid |
| | | | | | | | | (65° C.) |

Step 1 Myristic Acid Tank

| | | | | | | | | |
|---|---|---|---|---|---|---|---|---|
| Toluene (Solvent for | 92.14, 0.871 | 150 | ml | 1418 | mmol | 3 | mL/g | anhydrous (<0.1% |
| myristic acid) | g/mL | | | | | | | water) |

| | | | | | | | |
|---|---|---|---|---|---|---|---|
| Myristic acid | 228.4 | 55.92 | g | 244.8 | 1.1 | equiv | 99 wt % |

The amide (C-i) was synthesized according to the steps listed in Table 2.

TABLE 2
Method steps for synthesizing amide (C-1).

| | | |
|---|---|---|
| Unit | | |
| Op | Description of Lab Procedure | Notes/Observations |

Reaction Vessel #1, Myristic Acid Tank

| | | |
|---|---|---|
| 1. | Charge Toluene (3 mL/g) to reaction vessel #1 with | Water content of Toluene is <0.1%. |
| | reactor jacket set to 25(±5) ° C. | |
| 2. | Charge myristic acid (55.92 g, 245 mmol) to reaction | Myristic acid is a nicely flowable |
| | vessel #1 with jacket set to 25(±5) ° C. | flaky crystalline solid. |
| 3. | Heat the mixture to 35° C. (target) reactor temperature at | Myristic acid dissolves in 3 ml/g |
| | 1° C./min. | Toluene ~32° C. |
| 4. | Set agitation to 250 rpm and stir the mixture at 30° C. | |
| | (target) for 15 min (not less than (NLT) 15 min). | |

Reaction Vessel #2, Reaction

| | | |
|---|---|---|
| 5. | Charge Toluene (2 mL/g) to reaction vessel #2 with | |
| | reactor jacket set to 25 (±5) ° C. | |
| 6. | Charge 1,1′ Carbonyldiimidazole (CDI, 37.2 g, 223 | CDI in 2 ml/g toluene is a well- |
| | mmol) to reaction vessel #2 with jacket set to 25(±5) ° C. | mixed slurry at 25° C. |
| 7. | Set agitation to 250 rpm. | |
| 8. | Heat the mixture to 50° C. (target) at 1° C./min. | CDI in 2 ml/g Toluene is a thin |
| | | stirrable slurry at 50° C. |
| 9. | Transfer contents of reaction vessel #1 containing the | Transfer is expected to evolve CO₂, |
| | myristic acid solution to reaction vessel #2 with jacket | CO₂ evolution occurs as soon as first |
| | set to 50 (±5) ° C. over 30 mins (NLT 30 min). | drop of myristic acid solution reaches |
| | | reaction vessel #2. |
| | | Reaction mixture is clear solution |
| | | after all the myristic acid solution is |
| | | added. Subsequent steps run in |
| | | reaction vessel #2. |
| 10. | Rinse reaction vessel #1 and chase into reaction vessel | |
| | #2 with Toluene (0.5 mL/g). | |
| 11. | Stir the mixture at 50° C. (target) for 1 hour (NLT 1 | Mixture is clear homogenous |
| | hour). | solution. |
| 12. | Charge 1-tetradecylamine, (50 g, 227 mmol) to reaction | 1-tetradecylamine can be melted at |
| | vessel #2 with jacket set to 50 (±5) ° C. | 38-40° C. and charged as a low |
| | Visual check for dissolution of 1-tetradecylamine into | viscosity liquid. |
| | reaction mixture. | |
| 13. | Stir the mixture at 50° C. (target) for 1 hour (NLT 1 | Reaction kinetics are rapid, reaction |
| | hour). | is expected to reach in process |
| | | control (IPC) upon dissolution of the |
| | | amine into the reaction mixture. |
| 14. | Sample for reaction completion (gas chromatography - | Reaction mixture is stable for 40 hr at |
| | flame ionization detector area percent (AP) <2% 1- | 60° C. |
| | tetradecylamine) | |
| 15. | [Optional] Charge 1,1′ Carbonyldiimidazole (CDI, | In the event of IPC failure, an |
| | 3.72 g, 22.3 mmol) to reaction vessel #2 with jacket set | additional 0.1 eq CDI charge may be |
| | to 50(±5) ° C. | added to the pot followed by a one |
| | | hour stirring to drive the reaction to |
| | | completion. |
| 16. | [Optional] Stir the mixture at 50° C. (target) for 1 hour | |
| | (NLT 1 hour). | |

Reaction Vessel #2, Crystallization, filtration, and isolation

| | | |
|---|---|---|
| 17. | Charge Isopropanol (IPA, 5 mL/g) maintaining reactor | |
| | temperature NLT 45(±5) ° C. | |
| 18. | Cool the solution at 0.2° C./min (target) to 35° C. (target) | Primary nucleation occurred at ~37° C. |
| | reactor temperature for self-seeding. | |
| 19. | Stir the crystallized mixture at 35° C. (target) reactor | Cooling from 50° C. to produce |
| | temperature for 1.5 hours (NLT 1.5 hours). | spontaneous nucleation and holding |
| | | for 1.5 hours results in a thinly well- |
| | | mixed seed bed. |
| 20. | Charge Isopropanol (IPA, 5 mL/g) over NTL 1 hr | |
| 21. | Increased agitation to 425 rpm. | |
| 22. | Stir the crystallized mixture at 40° C. (target) reactor | |
| | temperature for 1 hour (NLT 1 hour). | |
| 23. | Cool the slurry to 20° C. (target) reactor temperature at | Cavern formation if high agitation |
| | 0.1° C./min with increased agitation to 625 rpm. | (625 rpm) was not used during this |
| | | cool-down. Slurry height |
| | | (H_{slurry}):Tank Diameter (D_{Tank}) of <1 |
| | | and. Impeller Diameter (Dᵢₘₚₑₗₗₑᵣ):Tank |
| | | Diameter (D_{Tank}) = 0.4-0.6. |
| 24. | Stir the crystallized mixture at 20° C. (target) reactor | |
| | temperature for 1 hours (NLT 1 hours). | |
| 25. | The slurry is filtered at 20° C. reactor temperature (NLT | In lab not more than (NMT) 0.4 barg |
| | 20° C.). | was used as wet cake tends to crack |
| | Filtration is stopped as soon as mother liquor just | and pull from the walls if any |
| | disappears to below cake surface. | deliquoring occurs. |
| | | Cake bulk density ~0.143-0.177 |
| | | g/cm³ |
| | | Minimum positive pressure to |
| | | perform this primary filtration. |
| 26. | Charge reactor with Toluene (2.0 ml/g) and | |
| | Isopropanol (4 ml/g) with jacket set to 20° C. (target). | |
| 27. | Charge wash to filter at 20° C. (target). | |
| | Filtration is stopped as soon as first wash just | |
| | disappears to below cake surface. | |
| 28. | Charge reactor with Toluene (6 ml/g) with jacket set to | |
| | 20° C. (target). | |
| 29. | Charge wash to filter at 20° C. (target). | |
| 30. | Dry solids under vacuum at 45(±5) ° C. for 6 hours | Solids have been dried at −0.90 barg |
| | (NLT 6 hours). | vacuum at 50° C. for 87 hr without |
| | | decomposition or melting. |

### Example 2

Producing an amide (C-1) from myristic acid and 1-tetradecylamine.

An amide (C-1) was formed according to the Scheme E1b. Oxalyl chloride (25.35 mmol, 1.5 eq. 3.22 g) was added at room temperature (RT) to a solution of myristic acid (3.86 g, 16.9 mmol) in benzene (40 mL) and dimethylformamide (DMF) (1 drop). The mixture was stirred at RT for 1.5 h. then heated at 60° C. for 30 min. The mixture was concentrated to form a residual. The residue formed was taken up in toluene and concentrated again. The residual oil (light yellow) was taken in 20 mL of benzene and added via syringe to a solution of 1-Tetradecylamine (2.86 13.4 mmol) and triethylamine (3.53 mL, 1.5 eq) in benzene (40 mL) at 10° C. After addition, the resulting mixture was stirred at RT overnight. The reaction mixture was diluted with water and was adjusted to pH 6-7 with 20% H₂SO₄. The mixture was filtered and washed with water. A pale solid was obtained. The crude product was recrystallized from methanol. This gave the desired product as an off-white solid (5.65 g, 13 mmol, 100%).

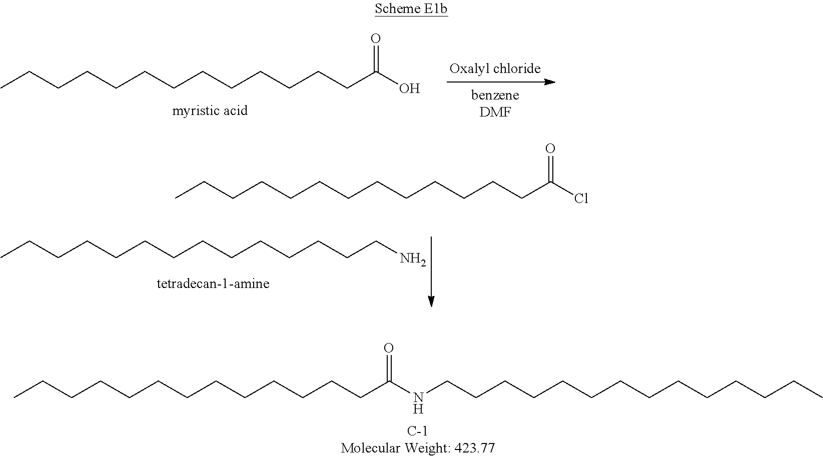 [SMILES: CCCCCCCCCCCCCC(=O)O ; CCCCCCCCCCCCCC(=O)Cl ; CCCCCCCCCCCCCCN ; CCCCCCCCCCCCCCNC(=O)CCCCCCCCCCCCC]

### Example 3

Producing an amine (C-2), and a succinate salt of the amine (C-2)

The amide (C-1), was reduced to form an secondary amine (C-2), and a salt of the secondary amine, according to the Scheme E2.

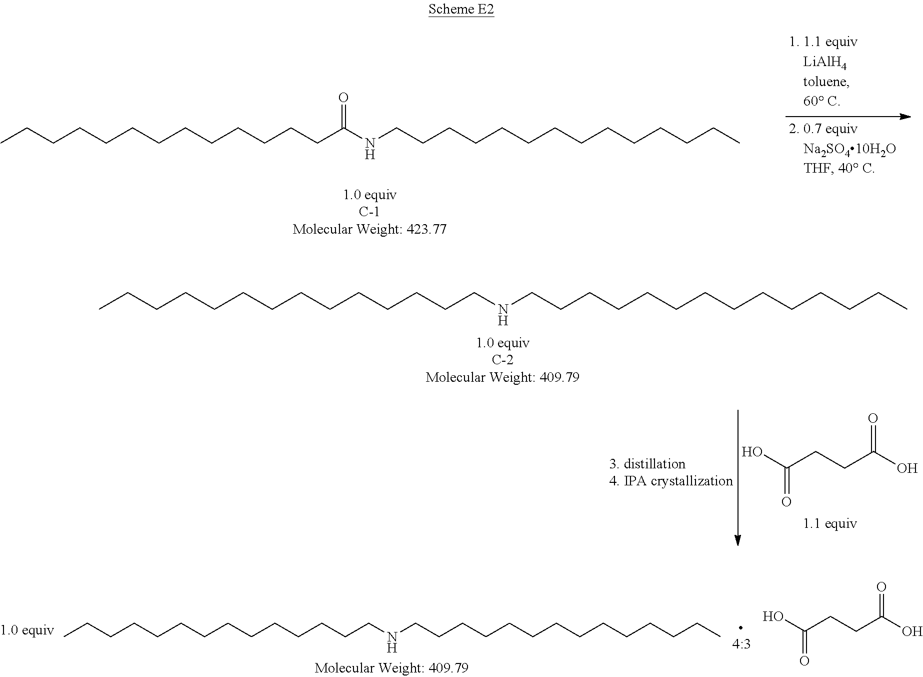

Material used for reducing the amide (C-i) and producing the amine (C-2), and producing the succinate salt of (C-2) is listed in Table 3. Equiv. and eq are used for equivalent.

TABLE 3
Material used.

| | | | | Molar | |
|---|---|---|---|---|---|
| | MW or | Weight or | | Ratio or | |
| Materials | Density | Volume | mmol | ml/g | Comments |

Step 2 Reaction Tank

| | | | | | | | | |
|---|---|---|---|---|---|---|---|---|
| Toluene (reaction | 92.14, 0.871 | 400 | mL | 3781 | mmol | 8 | mL/g | (<0.1 wt % water) |
| solvent) | g/mL | | | | | | | |
| Amide (C-1) | 423.8 | 50 | g | 118 | mmol | 50 | g | Typically ~95 wt %, reagent |
| Limiting Reagent | | | | | | | | charges are potency- |
| (LR) | | | | | | | | adjusted |
| LiAlH₄ (2M in | 37.95, 0.905 | 58.76 | g | 130 | mmol | 1.1 | equiv | |
| THF) | g/ml | | | | | | | |
| THF (chase solvent) | 72.11, 0.886 | 50 | mL | 614 | mmol | 1 | ml/g | (<0.1 wt % water) |
| | g/mL | | | | | | | |

Step 2 Quench Tank

| | | | | | | | | |
|---|---|---|---|---|---|---|---|---|
| THF (solvent for | 72.11, 0.886 | 150 | ml | 1843 | mmol | 3 | ml/g | (<0.1 wt % water) |
| sodium sulfate) | g/mL | | | | | | | |
| Sodium sulfate | 322.2 | 26.15 | g | 80.7 | mmol | 0.7 | equiv | Sodium sulfate decahydrate |
| decahydrate | | | | | | | | may be large and lumpy |
| | | | | | | | | solids. |
| THF (chase solvent) | 72.11, 0.886 | 250 | ml | 3072 | mmol | 5 | ml/g | (<0.1 wt % water) |
| | g/mL | | | | | | | |

Step 2 Distillation and Crystallization Tank

| | | | | | | | | |
|---|---|---|---|---|---|---|---|---|
| Isopropanol | 60.10, 0.786 | 250 | mL | 3270 | mmol | 6 | mL/g | (<0.1 wt % water) |
| (crystallization | mg/mL | | | | | | | |
| solvent) | | | | | | | | |
| Succinic acid | 118.09 | 15.02 | g | 127.2 | mmol | 1.1 | equiv | |
| Toluene (cake wash | 92.14, 0.871 | 300 | mL | 2836 | mmol | 2 | mL/g | (<0.1 wt % water) |
| solvent) | g/mL | | | | | | | |
| Isopropanol (cake | 60.10, 0.786 | 250 | mL | 3270 | mmol | 2 | mL/g | (<0.1 wt % water) |
| wash solvent) | mg/mL | | | | | | | |
| Amine (C-2) | 409.8 | 58.8 | g | 118 | mmol | 1 | equiv. | Theoretical potency of free |

| | |
|---|---|
| Tetamine NS | base = 81.7-83.6 wt %. |
| succinate salt) | Theoretical potency- |
| | adjusted yield is listed. |
| | Solution NMR indicates |
| | [(3.9-4.4):3] amine (C- |
| | 2):succinic acid |

Amine (C-2), and a succinate salt of Amine (C-2) was produced according to the steps listed in Table 4.

TABLE 4
Method steps for formation of amine (C-2), and a succinate salt of amine (C-2)

| | | |
|---|---|---|
| Unit | | |
| Op | Description of Lab Procedure | Notes/Observations |

Reactor #1, Reaction Tank

| | | |
|---|---|---|
| 1. | Charge Toluene (8 mL/g) to | Water content of Toluene used is <0.1 wt %. |
| | vessel #1 with jacket set to 25° C. | |
| 2. | Charge amide (C-1) (1 equiv.) to | Amide (C-1) is a free flowing powder. |
| | vessel #1 with jacket set to 25° C. | |
| 3. | Heat the mixture to 60° C. reactor | Kinetic solubility data of amide (C-1) shows >200 |
| | temperature at 1° C./min. | mg/ml solubility at 50° C., e.g., 5 ml Toluene/g LR is |
| | | sufficient to full dissolve amide (C-1) |
| 4. | Set agitation to 250 rpm and stir | Amide (C-1) density is lower than toluene and floats on |
| | the mixture at 60° C. reactor | the surface without agitation, stirring and time may be |
| | temperature (target) for NLT 15 | required for full dissolution of the amide. |
| | min. | |
| 5. | Charge Lithium Aluminum | LiAlH4 was charged in portions by mass. Earlier |
| | Hydride (1.1 equiv) to vessel #1 | portions generate the more hydrogen and produce more |
| | with target internal temperature | exotherm and the latter portions. |
| | 60 ± 5° C. | |
| 6. | Agitate the contents of vessel #1 | |
| | at 60° C. for NLT 2 h. | |
| 7. | Cool the contents of vessel #1 to | |
| | 25° C. over NLT 10 min. | |
| 8. | IPC: reaction conversion NMT | If IPC fails charge 0.2 equiv Lithium Aluminum |
| | 2% by gas chromatography. | Hydride at 60° C., agitate at 60° C. for 1 h and retake |
| | | IPC. |
| | | In an alternate experiment: Downstream steps have |
| | | been performed successfully with 1.3 equiv. Lithium |
| | | Aluminum Hydride and 1.3 equiv sodium sulfate |
| | | decahydrate followed by isolation of a 4:3 L-tartrate |
| | | salt. |
| 9. | Cool the contents of vessel #1 to | |
| | reactor temperature 25° C. over | |
| | NLT 10 min. | |

Reactor #2, Quench Tank

| | | |
|---|---|---|
| 10. | Charge THF (3 mL/g) to vessel | |
| | #2 with jacket set to 25° C. | |
| 11. | Agitate the contents of vessel #2 | Stirring was performed to fully suspend ~170 ml of |
| | at 550 rpm. | sodium sulfate decahydrate in 3 ml/g THF slurry. |
| | | Agitation was performed prior to addition of sodium |
| | | sulfate decahydrate. |
| 12. | Charge sodium sulfate | |
| | decahydrate (0.7 equiv) to vessel | |
| | #2 with jacket set to 25° C. | |
| 13. | Warm the contents of vessel #1 | |
| | to 40° C. at 1° C. /min. | |
| 14. | Transfer the contents of vessel | The reaction quench is exothermic and produces |
| | #1 to vessel #2 maintaining 40 ± | hydrogen gas. |
| | 5° C. in vessel #2 under vigorous | |
| | agitation. | |
| 15. | Rinse vessel #1 with THF (1 | |
| | mL/g) at 25° C. and transfer rinse | |
| | to vessel #2 | |
| 16. | Agitate the contents of vessel #2 | |
| | at 40° C. for NLT 1h. | |

Reactor #3, Crystallization Tank

| | | |
|---|---|---|
| 17. | Set the jacket of vessel #3 to | |
| | 40° C. | |
| 18. | Pass the slurry in vessel #2 | |
| | through a filter into vessel #3. | |
| 19. | Charge THF (5 mL/g) to vessel | |
| | #2 with jacket set to 40° C. and | |
| | agitate for NLT 15 min. | |
| 20. | Pass the contents of vessel #2 | |
| | through the waste cake and into | |
| | vessel #3. | |
| 21. | Concentrate the solution in | |
| | vessel #3 to 4 mL/g using 300 | |
| | mbar vacuum. | |
| 22. | Charge Isopropanol (6 ml/g) to | |
| | vessel #3 maintaining reactor | |
| | temperature 55 ± 5° C. | |
| 23. | Charge Succinic Acid (1.1 | Succinic acid is a nice flowable crystalline solid. |
| | equiv) to vessel #3 at 55 ± 5° C. | |
| 24. | Agitate the contents of vessel #3 | Dissolution of 1.1 eq succinic acid occurred in NMT 10 |
| | at 55 ± 5° C. for NLT 10 min | min on 50 g scale. |
| 25. | IPC: visual check for dissolution | |
| 26. | Cool vessel #3 to 35-40° C. at | 711145-312: Nucleation of the 4:3 succinate salt |
| | 1° C./min to initiate self-seeding. | occurred ~34° C.-37° C. |
| 27. | IPC: visual check for slurry. | Polarized light microscopy (PLM) of wet slurry at 35° C. |
| | | shows 500 × 25 μm needles. |
| 28. | Agitate the contents of vessel #3 | |
| | at 35° C. for NLT 2 h at 450 rpm. | |
| 29. | Cool the slurry to 20° C. at | |
| | 0.2° C./min at 300 rpm | |
| 30. | Agitate the contents of vessel #3 | PLM of wet slurry at 20° C. shows 175 × 45 μm needles. |
| | at 20° C. for NLT 1 h. | |
| 31. | Filter the slurry at 20° C. Stop | NMT 0.4 barg was used as cake compressibility testing |
| | filtration as soon as mother | has not been completed. |
| | liquor reaches the top surface of | Cake bulk density = 0.267 g/cm³ |
| | the cake. | |
| 32. | Charge Toluene (2.0 ml/g) and | |
| | Isopropanol (2 ml/g) to vessel #3 | |
| | with jacket set to 20° C. | |
| 33. | Charge wash to filter at 20° C. | |
| | Filtration is stopped as soon as | |
| | first was reaches the top surface | |
| | of the cake. | |
| 34. | Dry solids under vacuum at | Solid were dried at −0.9 barg vacuum at 50° C. for 15 hr. |
| | 45(±5) ° C. for 5 hours (NLT 5 | |
| | hours). | |

### Example 4

### Synthesizing a polymer conjugated lipid from the amine (C-2) succinate salt and a PEG acid (C-3)

A polymer conjugated lipid (C-4) synthesized from the succinate salt of the amine (C-2), according to Scheme E3a. The yield of C-4 ranged from 89 to 95%.

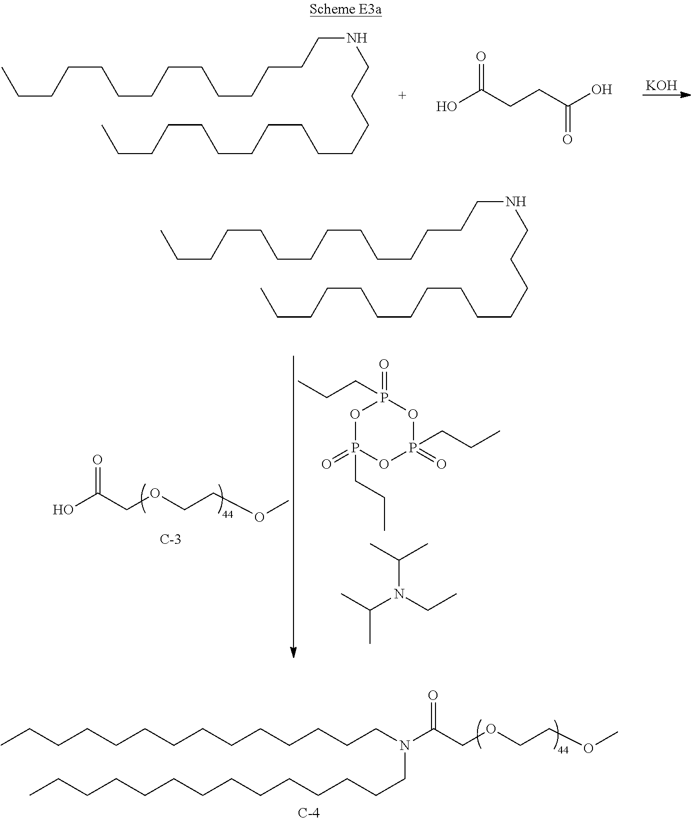

Material used for synthesis of the polymer conjugated lipid (C-4) from the salt of the amine (C-2), are listed in Table 5. Equiv. and eq are used for equivalent.

TABLE 5
Material used.

| | | | | | |
|---|---|---|---|---|---|
| | | | | Molar | |
| | MW or | Weight or | | Ratio or | |
| Materials | Density | Volume | mmol | ml/g | Comments |

Step: Forming the amine (C-2)from succinate salt of the amine (C-2)

| Toluene (salt break) | 92.14, 0.871 | 160 | mL | 1512 | 8 | mL/g | |
|---|---|---|---|---|---|---|---|
| | g/mL | | | | | | |
| amine (C-2):succinate salt | 409.8 | 6.39 | g | 12.8 | 1.3 | equiv. | 6:4 (C- |
| | | | | | | | 2):succinic |
| | | | | | | | acid salt, ~81- |
| | | | | | | | 84 wt % amine |
| Water (wash #1) | 18.02, 1.0 | 80 | mL | 4441 | 4 | L/kg | |
| | g/mL | | | | | | |
| Potassium hydroxide (wash #1) | 56.11 | 2.77 | g | 49.3 | 5.0 | equiv. | |
| Water (wash #2) | 18.02, 1.0 | 80 | mL | 4441 | 4 | L/kg | |
| | g/mL | | | | | | |
| Potassium hydroxide (wash #2) | 56.11 | 2.77 | g | 49.3 | 5.0 | equiv. | |
| Water (wash #3) | 18.02, 1.0 | 80 | mL | 4441 | 4 | L/kg | |
| | g/mL | | | | | | |
| Water (optional wash #1) | 18.02, 1.0 | 80 | mL | 4441 | 4 | L/kg | |
| | g/mL | | | | | | |
| Potassium hydroxide (optional | 56.11 | 2.77 | g | 49.3 | 5.0 | equiv. | |
| wash #1) | | | | | | | |
| Water (optional wash #2) | 18.02, 1.0 | 80 | mL | 4441 | 4 | L/kg | |
| | g/mL | | | | | | |
| Toluene (optional second | 92.14, 0.871 | 60 | mL | 567 | 3 | L/kg | |
| distillation #1) | g/mL | | | | | | |
| Free amine (C-2) in toluene | 409.8 | 92 g (total) | | 12.8 (amine) | 1.3 | equiv. | 5.7 theoretical |
| | | | | | | | wt % amine in |
| | | | | | | | toluene |
| Dipotassium succinate | 194.27 | 1.66 | g | 8.53 | 0.87 | equiv. | |
| (byproduct) | | | | | | | |
| Water (byproduct) | 18.02, 1.0 | 0.31 | mL | 17.1 | 1.73 | equiv. | |
| | g/mL | | | | | | |

Step: Formation of lipid (C-4)from (C-2)

| | | | | | | | |
|---|---|---|---|---|---|---|---|
| PEG acid (C-3) | 2028.4 (average) | 20 | g | 9.86 mmol | 1.0 | equiv | |
| Toluene (reaction solvent) | 92.14, 0.871 | 160 | mL | 1512 | 8 | mL/g | |
| | g/mL | | | | | | |
| Toluene (optional second | 92.14, 0.871 | 60 | mL | 567 | 3 | mL/g | |
| distillation #2) | g/mL | | | | | | |
| Diisopropylethylamine | 129.24, 0.741 | 6.88 | mL | 39.4 | 4.0 | equiv. | |
| (Hunig's base) | g/mL | | | | | | |
| 1-Propanephosphonic Acid | 318.18, 1.069 | 11.74 | mL | 19.7 | 2.0 | equiv. | 50 wt % |
| Cyclic Anhydride (T3P | g/mL | | | | | | solution in |
| coupling agent) | | | | | | | Ethyl Acetate |
| Toluene (chase #1) | 92.14, 0.871 | 20 | mL | 189 | 1 | L/kg | |
| | g/mL | | | | | | |
| 1-Propanephosphonic Acid | 318.18, 1.069 | 1.17 | mL | 1.97 | 0.2 | equiv. | 50 wt % |
| Cyclic Anhydride (T3P | g/mL | | | | | | solution in |
| coupling agent) | | | | | | | Ethyl Acetate |
| Water | 18.02, 1.0 | 86 | mL | 4774 | 4.3 | L/kg | Pre-prepared |
| | g/mL | | | | | | solution in |
| Potassium carbonate | 138.21 | 6.81 | g | 49.3 | 5.0 | equiv. | reactor #4 |
| Sodium chloride | 58.44 | 23.62 | g | 404.26 | 41 | equiv. | |
| Toluene (distillation) | 92.14, 0.871 | 200 | mL | 1891 | 10 | L/kg | |
| | g/mL | | | | | | |
| Toluene (optional second | 92.14, 0.871 | 200 | mL | 1891 | 10 | L/kg | |
| distillation #3) | g/mL | | | | | | |
| Toluene (chase #2) | 92.14, 0.871 | 20 | mL | 189 | 1 | L/kg | |
| | g/mL | | | | | | |
| Lipid (C-4)in crude toluene | 2420.2 | 23.86 | g | 9.86 mmol | 1.0 | equiv. | 23.86 g as |
| solution | | | | | | | maximum |
| | | | | | | | theoretical |
| | | | | | | | yield in crude |
| | | | | | | | toluene |
| | | | | | | | solution |
| Potassium bicarbonate | | | | | | | |
| (byproduct) | | | | | | | |
| monopotassium | 161.16 | 3.18 | g | 19.72 | 2.0 | equiv. | Up to 2.2 |
| mono(propylphosphonate) | | | | | | | equiv. |
| (byproduct) | | | | | | | depending on |
| | | | | | | | optional |
| | | | | | | | additional |
| | | | | | | | T3P charge |
| monopotassium | 267.22 | 5.26 | g | 19.72 | 2.0 | equiv. | Up to 2.2 |

| | | | | | | |
|---|---|---|---|---|---|---|
| mono(dipropyldiphosphonate) | | | | | | equiv. |
| (byproduct) | | | | | | depending on |
| | | | | | | optional |
| | | | | | | additional |
| | | | | | | T3P charge |
| N-ethyl-N-isopropylpropan-2- | 130.25 | 5.12 | 39.44 | 4.0 | equiv | |

aminium (byproduct)

The amine (C-2) was formed from the succinate salt of the amine (as produced in Example 2), and the polymer conjugated lipid (C-4) was formed from the reformed amine (C-2), according to the steps listed in Table 6.

TABLE 6
Method steps for reforming amine (C-2), and forming
lipid (C-4) from reformed amine (C-2).

| | | |
|---|---|---|
| Unit | | |
| Op | Description of Lab Procedure | Notes/Observations |

Reactor #1 (salt break)

| | | |
|---|---|---|
| 1. | Set reactor jacket temperature (Ti) to 25° C. | |
| 2. | To reactor #1, charge Toluene (salt break) (8 | |
| | L/kg). | |
| 3. | To reactor #1, charge amine (C-2) succinate | Amine (C-2) succinate salt is a fluffy, |
| | salt. | white powder. |
| 4. | Set agitation to 250 rpm. | Reaction is a white slurry. |
| 5. | Warm contents of reactor #1 to 40° C. (Tr). | |
| 6. | To reactor #1, charge Water (wash #1) (4 | |
| | L/kg). | |
| 7. | To reactor #1, charge solid Potassium | No off-gassing, 1-2° C. exotherm. pH of |
| | Hydroxide (wash #1) (5.0 equiv.). | the resulting aqueous layer is 14. |
| 8. | Stir biphasic solution for NLT 2 hours. | Mixture remains a white slurry for up |
| | | to ~15 minutes before becoming a more |
| | | clear solution. |
| 9. | (For information only (FIO)) Pull sample of | |
| | biphase. | |
| 10. | Halt agitation, and allow layers to separate for | |
| | NLT 1 hour. | |
| 11. | Drain and discard the bottom, aqueous layer, | |
| | keeping the interface with the organic layer. | |
| 12. | To reactor #1, charge Water (wash #2) (4 | |
| | L/kg). | |
| 13. | To reactor #1, charge solid Potassium | No off-gassing, 1-2° C. exotherm. pH of |
| | Hydroxide (wash #2) (5.0 equiv.). | the resulting aqueous layer is 14. |
| 14. | Stir biphasic solution for NLT 2 hours. | |
| 15. | (FIO) Pull sample of biphase. | |
| 16. | Halt agitation, and allow layers to separate for | |
| | NLT 1 hour. | |
| 17. | Drain and discard the bottom, aqueous layer, | |
| | keeping the interface with the organic layer. | |
| 18. | To reactor #1, charge Water (4 L/kg). | Clear, colorless biphasic solution. pH of |
| | | resulting aqueous layer is 11. |
| 19. | Stir biphasic solution for NLT 2 hours. | |
| 20. | (FIO) Pull sample of biphase. | |
| 21. | Stop agitation, and allow layers to separate for | |
| | NLT 1 hour. | |
| 22. | Drain and discard the bottom, aqueous layer, | |
| | keeping the interface with the aqueous layer. | |
| 23. | (IPC) Succinic acid NMT 40 ppm by LC-MS. | If IPC fails, follow steps 25-34 and then |
| | | repeat IPC. |
| 24. | (optional) To reactor #1, charge Water | |
| | (optional wash #1) (4 L/kg). | |
| 25. | (optional) To reactor #1, charge solid | No off-gassing, 1-2° C. exotherm. pH of |
| | Potassium Hydroxide (optional wash #1) (5.0 | the resulting aqueous layer is 14. |
| | equiv.). | |
| 26. | (optional) Stir biphasic solution for NLT 2 | |
| | hours. | |
| 27. | (optional) (FIO) Pull sample of biphase. | |
| 28. | (optional) Stop agitation, and allow layers to | |
| | separate for NLT 1 hour. | |
| 29. | (optional) Drain and discard the bottom, | |
| | aqueous layer, keeping the interface with the | |
| | organic layer. | |
| 30. | (optional) To reactor #1, charge Water | |
| | (optional wash) (4 L/kg). | |
| 31. | (optional) Stir biphasic solution for NLT 2 | |
| | hours. | |
| 32. | (optional) (FIO) Pull sample of biphase. | |
| 33. | (optional) Stop agitation, and allow layers to | |
| | separate for NLT 1 hour. | |
| 34. | (optional) Drain and discard the bottom, | |
| | aqueous layer, keeping the interface with the | |
| | aqueous layer. | |
| 35. | Distill to 5 L/kg with respect to PEG acid (100 | 150 mbar, distillation commences at ~Tr = |
| | mL) under vacuum at a temperature no greater | 55° C., distillation ended at Tr = 59° C., |
| | than 70° C. (Tr). | Tj − Tr = 30° C. |
| 36. | Cool to 35° C. at 1° C./min. | |
| 37. | Disconnect the vessel from vacuum. | Clear, colorless solution at Tr = ~30° C. |
| 38. | (IPC) KF analysis (water NMT 0.05 wt %). | |
| 39. | Hold reactor #1 mixture until completion of | |
| | step 47. | |

Reactor #2 (PEG acid activation)

| | | |
|---|---|---|
| 40. | Set Tj to 25° C. | |
| 41. | To reactor #2, charge Toluene (reaction | |
| | solvent) (8 L/kg). | |
| 42. | To reactor #2, charge PEG acid (C-3) (20 g). | |
| 43. | Set agitation to 250 rpm. | |
| 44. | Warm to 45° C. (Tr). | Clear, colorless solution attained before |
| | | Tr reaches ~29-30° C. |
| 45. | Distill solution down to 4 L/kg under vacuum | 150 mbar, distillation commences at ~Tr = |
| | at a temperature no greater than 70° C. (Tr). | 55° C., distillation ended at Tr = 59° C., |
| | | Tj − Tr = 30° C. |
| 46. | Set Tr to 25° C. | |
| 47. | Disconnect the vessel from vacuum. | |
| 48. | (IPC) KF analysis (water NMT 0.05 wt %). | If IPC fails, charge Toluene (optional |
| | | second distillation #2) (3 L/kg) and distill |
| | | down to 4 L/kg. Repeat IPC check. |
| 49. | Warm to 35° C. (Tr). | |
| 50. | To reactor #2, charge Diisopropylethylamine | Clear, colorless, mobile solution. |
| | (base) (4.0 equiv.). | |
| 51. | To reactor #2, charge 1-Propanephosphonic | No off-gassing or exotherm. Clear, |
| | Acid Cyclic Anhydride (50 Mass %) in Ethyl | colorless, mobile solution. |
| | Acetate (coupling agent) (2.0 equiv). | |
| 52. | Agitate contents of reactor #2 for NLT 1 hour. | Clear, colorless, mobile solution. |
| 53. | Charge contents of reactor #2 into reactor #1. | No off-gassing or exotherm. Clear, |
| | | colorless, mobile solution. |
| 54. | Into reactor #2, charge Toluene (chase #1) (1 | |
| | L/kg). | |
| 55. | Set agitation to 500 rpm. | |
| 56. | Stir NLT 5 minutes. | |
| 57. | Charge contents of reactor #2 into reactor #1. | |

Reactor #1 (after transfer of reactor #2 solution into #1)

| | | |
|---|---|---|
| 58. | Agitate resulting reactor #1 solution for NLT | |
| | 1 hour. | |
| 59. | (IPC) Starting material NMT 2 wt % by ultra- | |
| | performance liquid chromatography - charged | |
| | aerosol detection. | |
| 60. | Cool to 25° C. (Tr) at 1° C./min. | All crude reaction components are soluble |
| | | at room temperature. |
| 61. | Set agitation to 250 rpm. | |
| 62. | To reactor #1, charge the aqueous solution | Potassium Carbonate (5.0 equiv.), Sodium |
| | prepared in reactor #3. | Chloride (41 equiv.), and Water (4.3 |
| | | L/kg). |
| | | pH of this aqueous solution is ~13 |
| | | No off-gassing or exotherm. |
| 63. | Stir reactor #1 for NLT 30 minutes. | |
| 64. | Stop agitation, and allow layers to separate | |
| | (NLT 3 hours). | |
| 65. | Drain and discard the bottom, aqueous layer. | The pH of the resulting aqueous layer |
| | | is ~9. |
| 66. | Distill wet toluene solution to 5 L/kg under | 150 mbar of vacuum was used in lab. |
| | vacuum at a temperature no greater than 70° C. | Distillation starts at 45° C. and ends at |
| | (Tr). | 57° C. |
| 67. | Disconnect the vessel from vacuum. | |
| 68. | Charge Toluene (distillation) (10 L/kg) distill | |
| | down to 5 L/kg. | |
| 69. | Cool to 25° C. (Tr) at 1° C./min. | |
| 70. | (IPC) KF analysis (water NMT 0.05 wt %). | If IPC fails, charge Toluene (optional |
| | | second distillation #3) (10 L/kg) distill |
| | | down to 5 L/kg. Repeat IPC check. |
| 71. | Stir for NLT 2 hours. | |
| 72. | Filter through Nutsche into reactor #4. | |
| 73. | (If resin chromatography is used, perform step | |
| | 73 before continuing on to step 74) Perform | |
| | solvent swap to 20 L/kg EtOH. | |
| 74. | Drum contents of reactor #4. | |
| 75. | Into reactor #1, charge Toluene (chase #2) (1 | |
| | L/kg). | |
| 76. | Set agitation to 500 rpm. | |
| 77. | Stir NLT 5 minutes. | |
| 78. | Transfer contents of reactor #1 through | |
| | Nutsche into reactor #4. | |
| 79. | Drum contents of reactor #4. | |

### Example 5

### Synthesizing a polymer conjugated lipid (C-4) from the amine (C-2) and mPEG-NHS

A polymer conjugated lipid (C-4) was synthesized according to scheme E3b. A solution of mPEG-NHS (from NOF, 5.0 mmol, 9.97 g, PEG MW approx. 2,000, n=about 45) in DCM (120 mL) was added to a solution of the secondary amine, N-Tetradecyl-l-tetradecanamin, (C-2) (7 mmol, 2.87 g) and triethylamine (30 mmol, 4.18 mL) in dichloromethane (DCM) (100 mL). After 24 h the reaction solution was washed with water (300 mL). The aqueous phase was extracted twice with DCM (100 mL×2). DCM extracts were combined and washed with brine (100 mL). The organic phase was dried over sodium sulfate, filtered, and concentrated partially. The partially concentrated solution (approximately 300 mL) was cooled at approximately −15° C. Filtration gave a white solid (1.030 g, the unreacted starting amine). To the filtration was added Et₃N (1.6 mmol, 0.222 mL, 4 eq) and acetic anhydride (1.6 mmol, 164 mg). The mixture was stirred at RT for 3 h and then concentrated to a solid. The residual solid was purified by column chromatography on silica gel (0-8% methanol in DCM). This gave the desired product as a white solid (9.211 g).

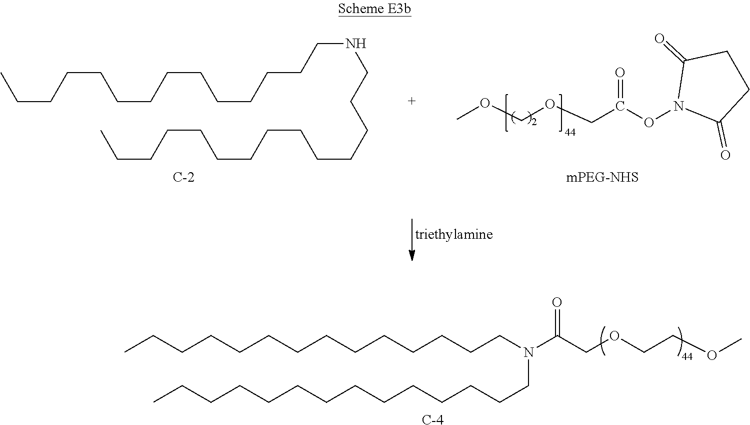

Embodiment 1. A method for producing a compound having a chemical formula of Formula I

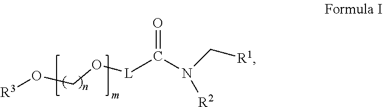

wherein R1 and R2 are independently a i) linear or branched or cyclic, ii) saturated or unsaturated, and iii) substituted or unsubstituted hydrocarbon group comprising 8 to 20 carbon atoms,

R3 is a hydrocarbon group,

n is an integer from 2 to 5,

m is an integer from 30 to 70, and

L is a linker,

the method comprising:
a) forming an amide having a chemical formula of R1—C(O)—NH—R2 from a fatty acid having a chemical formula of R1—COOH and a primary amine having a chemical formula of R2—NH₂;
b) contacting the amide with a reducing agent to form a secondary amine having a chemical formula of R1—CH₂—NH—R2; and
c) contacting the secondary amine with a polyolefin-glycol compound to form the compound of Formula I.

Embodiment 2. The method of Embodiment 1, wherein the fatty acid is contacted with 1,1′-Carbonyldiimidazole (CDI) to form a N-acyl imidazole having the chemical formula of of R1—C(O)—C₃N₂H₄, and the N-acyl imidazole is contacted with the primary amine to form the amide.

Embodiment 3. The method of Embodiment 2, wherein the fatty acid and CDI have a molar ratio of 1:1.2 to 1.2:1.

Embodiment 4. The method of any one of Embodiments 2 to 3, wherein contacting the fatty acid with CDI is performed at a temperature of 40° C. to 60° C.

Embodiment 5. The method of any one of Embodiments 2 to 4, wherein the fatty acid and CDI are contacted in the presence of toluene.

Embodiment 6. The method of any one of Embodiments 1 to 5, wherein the N-acyl imidazole and the primary amine have a molar ratio of 0.9:1 to 1:0.9.

Embodiment 7. The method of any one of Embodiments 2 to 6, wherein the amount of primary amine contacted with the N-acyl imidazole is 0.85 to 1.2 moles of primary amine per mole of the fatty acid used to form the N-acyl imidazole.

Embodiment 8. The method of any one of Embodiments 1 to 7, wherein at least a portion of the primary amine is in a melted form.

Embodiment 9. The method of any one of Embodiments 1 to 8, wherein the N-acyl imidazole and the primary amine are contacted at a temperature of 40° C. to 60° C. Embodiment 10. The method of Embodiment 1, wherein the fatty acid is contacted with an oxychloride to form an acyl chloride having a chemical formula of R1—C(O)—Cl, and the acyl chloride is contacted with the primary amine to form the amide, wherein the oxychloride is selected from thionyl chloride, phosphoryl chloride, oxalyl chloride, and any combinations thereof.

Embodiment 11. The method of Embodiment 10, wherein the fatty acid and the oxychloride have a molar ratio of 1:0.8 to 1:2.

Embodiment 12. The method of any one of Embodiments 10 to 11, wherein the fatty acid and the oxychloride are contacted in the presence of benzene and dimethylformamide.

Embodiment 13. The method of any one of Embodiments 10 to 12, wherein the fatty acid and the oxychloride are contacted at a temperature of 20° C. to 75° C.

Embodiment 14. The method of any one of Embodiments 10 to 13, wherein the oxychloride is oxalyl chloride.

Embodiment 15. The method of any one of Embodiments 10 to 14, wherein the acyl chloride and primary amine are contacted at a temperature of 2° C. to 20° C.

Embodiment 16. The method of any one of Embodiments 10 to 15, wherein the acyl chloride and primary amine are contacted in the presence of benzene and triethylamine.

Embodiment 17. The method of any one of Embodiments 10 to 16, wherein the amount of primary amine contacted with the acyl chloride is 0.6 to 1.2 moles of primary amine per mole of the fatty acid used to form the acyl chloride.

Embodiment 18. The method of any one of Embodiments 1 to 17, further comprising crystallizing the amide from an amidation-product mixture formed in step (a), and using the crystallized amide as at least a portion of the amide in step (b).

Embodiment 19. The method of Embodiment 18, wherein crystallizing the amide comprises adding isopropanol to the amidation-product mixture to form a crystallization mixture and cooling the crystallization mixture.

Embodiment 20. The method of Embodiment 19, wherein crystallizing the amide s comprises:
contacting isopropanol with the amidation-product mixture at a temperature greater than 40° C. and at or below 60° C. to form the crystallization mixture;
cooling the crystallization mixture to a temperature of 30° C. to 40° C. to form a slurry containing amide crystals;
maintaining the slurry at a temperature of 30° C. to 40° C., with continuous, periodic, or occasional stirring for at least 1 hour;
cooling the slurry to a temperature of 15° C. to 25° C. with continuous, periodic, or occasional stirring;
maintaining the slurry at a temperature of 15° C. to 25° C. with continuous, periodic, or occasional stirring for at least 0.5 hour; and separating the amide crystals from the slurry.

Embodiment 21. The method of Embodiment 20, wherein the slurry is cooled to a temperature of 15° C. to 25° C. with continuous stirring at 600 rpm or above.

Embodiment 22. The method of any one of Embodiments 18 to 21, wherein the amide crystals are separated from the slurry by filtration.

Embodiment 23. The method of Embodiment 22, further comprising washing the filtered amide crystals with toluene and/or isopropanol, and drying the washed crystals.

Embodiment 24. The method of Embodiment 23, wherein the amide crystals are dried at a temperature of 40° C. to 50° C.

Embodiment 25. The method of any one of Embodiments 18 to 24, wherein the amide crystallization process is performed in a reactor having a diameter D, and the reactor comprises an impeller having a diameter DI, and DI:D is 0.35:1 to 0.65:1 Embodiment 26. The method of Embodiment 25, wherein the slurry in the reactor has a height H, and H is less than D.

Embodiment 27. The method of any one of Embodiments 18 to 26, wherein the amidation-product mixture comprises less than 4% by weight of the starting primary amine.

Embodiment 28. The method of any one of Embodiments 1 to 27, wherein the reducing agent is a hydride.

Embodiment 29. The method of Embodiment 28, wherein the hydride is lithium aluminum hydride.

Embodiment 30. The method of any one of Embodiments 1 to 29, wherein the amide and the reducing agent have a molar ratio of 1:1 to 1:3.

Embodiment 31. The method of any one of Embodiments 1 to 30, wherein in step (b) an amide solution comprising the amide is contacted with a reducing agent solution comprising the reducing agent.

Embodiment 32. The method of Embodiment 31, wherein the amide solution further comprises toluene and/or the reducing agent solution further comprises 2-methyl tetrahydrofuran (THF) and/or THF.

Embodiment 33. The method of any one of Embodiments 31 to 32, wherein the amide solution is formed by contacting crystals of the amide with toluene.

Embodiment 34. The method of any one of Embodiments 1 to 33, wherein contacting of the amide and the reducing agent is performed at a temperature of 50° C. to 75° C.

Embodiment 35. The method of any one of Embodiments 1 to 34, wherein step (b) the amide is reduced to form the secondary amine, and the step (b) further comprises quenching the reduction of the amide by adding sodium sulfate.

Embodiment 36. The method of Embodiment 35, wherein quenching the reduction of the amide comprises:
contacting a reduction-product mixture formed in step (b) with a slurry comprising sodium sulfate at a temperature of 35° C. to 45° C. to form a quenched reduction-product mixture and residual sodium sulfate;
separating at least a portion of the residual sodium sulfate from the quenched reduction-product mixture to form a separated reduction-product mixture comprising the secondary amine.

Embodiment 37. The method of Embodiment 36, wherein the reduction-product mixture comprises less than 4% by weight of the starting amide.

Embodiment 38. The method of any one of Embodiments 35 to 37, wherein 0.5 to 2 moles of sodium sulfate per mole of amide is added.

Embodiment 39. The method of any one of Embodiments 36 to 38, wherein the slurry comprising sodium sulfate further comprises THF and/or toluene.

Embodiment 40. The method of any one of Embodiments 36 to 39, wherein the at least a portion of the residual sodium sulfate is separated from the quenched reduction-product mixture by filtration, wherein the separated reduction-product mixture is formed as a filtrate.

Embodiment 41. The method of any one of Embodiments 1 to 40, further comprising forming a crystallized salt of the secondary amine.

Embodiment 42. The method of Embodiment 41, wherein the crystallized salt of the secondary amine is formed by a process comprising,
contacting the secondary amine with an acid to form a salt-forming solution comprising a salt of the secondary amine, and
cooling the salt-forming solution to form the crystallized salt of the secondary amine.

Embodiment 43. The method of any one of Embodiments 41 or 42, wherein the crystallized salt of the secondary amine is formed by a process comprising:
contacting the secondary amine with isopropanol and an acid at a temperature of 50° C. to 60° C. to form a salt-forming solution comprising a salt of the secondary amine;
cooling the salt-forming solution to 30° C. to 45° C. to form salt crystals;
maintaining the salt-forming solution at 30° C. to 45° C. for at least 1 hour;
cooling the salt-forming solution to 15° C. to 25° C.;
separating the salt crystals from the salt-forming solution.

Embodiment 44. The method of Embodiment 43, wherein the salt crystals are separated from the salt-forming solution by filtering, wherein the crystallized salt is obtained as filtered residue.

Embodiment 45. The method of Embodiment 44, further comprising washing and drying the filtered residue to form a dried, crystallized salt of the secondary amine.

Embodiment 46. The method of Embodiment 45, wherein the filtered residue is washed with a toluene and/or isopropanol solution.

Embodiment 47. The method of Embodiment 46, wherein volume % ratio of the toluene and isopropanol in the toluene and/or isopropanol solution is 0.9:1 to 1:0.9.

Embodiment 48. The method of any one of Embodiments 45 to 47, wherein the filtered residue is dried at a pressure of 0 to 0.2 bar and/or a temperature of 40° C. to 50° C.

Embodiment 49. The method of any one of Embodiments 42 to 48, wherein the acid is hydrochloric acid, hydrobromic acid, hydroiodic acid, sulfuric acid, acetic acid, methanesulfonic acid, toluenesulfonic acid, (1R)-(-)-10-camphorsulfonic acid, 1,2-ethanedisulfonic acid, oxalic acid, dibenzoyl-L-tartaric acid, phosphoric acid, L-tartaric acid, maleate, fumaric acid, succinic acid, and/or malonic acid

Embodiment 50. The method of any one of Embodiments 42 to 49, wherein the acid is succinic acid.

Embodiment 51. The method of any one of Embodiments 45 to 50, wherein the secondary amine is reformed from the dried, crystallized salt of the secondary amine, and the reformed secondary amine is used in step (c).

Embodiment 52. The method of Embodiment 51, wherein the secondary amine is reformed from the dried, crystallized salt by contacting the dried, crystallized salts with a base.

Embodiment 53. The method of Embodiment 51, wherein the secondary amine is reformed from the dried, crystallized salt of the secondary amine by a process comprising:
contacting the dried, crystallized salt of the secondary amine with an organic solvent to form a salt solution,
washing the salt solution with a base and water to form a washed organic solution comprising the secondary amine; and distilling the washed organic solution to form a distilled organic solution.

Embodiment 54. The method of Embodiment 53, wherein the salt solution is washed with the base and/or water more than once.

Embodiment 55. The method of any one of Embodiments 53 to 54, wherein the dried, crystallized salt is contacted with toluene to form the salt solution.

Embodiment 56. The method of any one of Embodiments 53 to 55, wherein the washed organic solution comprises less than 100 μg per mL of the acid.

Embodiment 57. The method of any one of Embodiments 53 to 56, wherein the washed organic solution is distilled at a pressure of 0 to 0.3 bar and/or a temperature at or below 70° C. to form the distilled organic solution.

Embodiment 58. The method of any one of Embodiments 53 to 57, wherein the distilled organic solution comprises less 0.05 wt. % of water.

Embodiment 59. The method of any one of Embodiments 53 to 58, wherein the base is NaOH and/or KOH.

Embodiment 60. The method of any one of Embodiments 53 to 59, wherein the reformed secondary amine in the distilled organic phase is used in step (c).

Embodiment 61. The method of any one of Embodiments 1 to 60, wherein the polyolefin-glycol compound is a polyolefin glycol acid having a chemical formula of HOOC—L—(O(CH₂)ₙ)ₘ—OR₃.

Embodiment 62. The method of Embodiment 61, wherein the secondary amine and the polyolefin glycol acid have a molar ratio of 1:1.2 to 1:1.5.

Embodiment 63. The method of any one of Embodiments 61 to 62, wherein the polyolefin glycol acid is activated by contacting the polyolefin glycol acid with an organic base and a coupling agent to form a coupling solution comprising an activated polyolefin-glycol compound, and the coupling solution is contacted with the secondary amine.

Embodiment 64. The method of Embodiment 63, wherein the organic base is a tertiary amine.

Embodiment 65. The method of Embodiment 64, wherein the tertiary amine is diisopropylethylamine.

Embodiment 66. The method of any one of Embodiments 63 to 65, wherein the coupling agent is 1-propanephosphonic acid cyclic anhydride.

Embodiment 67. The method of any one of Embodiments 63 to 66, wherein the coupling solution is colorless.

Embodiment 68. The method of any one of Embodiments 63 to 67, wherein the coupling solution is formed by contacting the polyolefin glycol acid and the organic base at a molar ratio of 1:3.5 to 1:4.5.

Embodiment 69. The method of any one of Embodiments 63 to 68, wherein the coupling solution is formed by contacting the polyolefin glycol acid and the coupling agent at a molar ratio of 1:1.8 to 1:2.2.

Embodiment 70. The method of any one of Embodiments 63 to 69, wherein the coupling solution is formed by contacting the polyolefin glycol acid with an organic solvent to form a polyolefin glycol solution, distilling the polyolefin glycol solution to form a distilled polyolefin glycol solution, and contacting the distilled polyolefin glycol solution with the base and coupling agent to form the coupling solution.

Embodiment 71. The method of Embodiment 70, wherein the polyolefin glycol solution is distilled at a pressure of 0 to 0.2 bar and/or a temperature at or below 70° C. Embodiment 72. The method of any one of Embodiments 70 to 71, wherein the distilled polyolefin glycol solution contains less than 0.05 wt. % of water.

Embodiment 73. The method of any one of Embodiments 70 to 72, wherein the polyolefin glycol acid is contacted with toluene to form the polyolefin glycol solution.

Embodiment 74. The method of any one of Embodiments 63 to 73, wherein in step (c) the coupling solution is contacted with a reformed secondary amine.

Embodiment 75. The method of any one of Embodiments 63 to 74, wherein in step (c) the coupling solution is contacted with a reformed secondary amine from the distilled organic solution.

Embodiment 76. The method of any one of Embodiments 1 to 75, wherein in step (c) the compound of Formula I is formed at a temperature of 20° C. to 45° C.

Embodiment 77. The method of any one of Embodiments 1 to 60, wherein the polyolefin-glycol compound is a N-hydroxylsuccinimide (NHS) functionalized polyolefin glycol, and has a chemical formula of NHS—O(O)C—L—(O(CH₂)ₙ)ₘ—OR₃.

Embodiment 78. The method of Embodiment 77, wherein the NHS functionalized polyolefin glycol is contacted with the secondary amine at a molar ratio 0.6:1 to 1.2:1.

Embodiment 79. The method of any one of Embodiments 77 to 78, wherein the NHS functionalized polyolefin glycol is contacted with the secondary amine in presence of a tertiary amine.

Embodiment 80. The method of Embodiment 79, wherein the tertiary amine is triethylamine.

Embodiment 81. The method of any one of Embodiments 77 to 80, wherein the NHS functionalized polyolefin glycol is contacted with the secondary amine at a temperature of 20° C. to 45° C.

Embodiment 82. The method of any one of Embodiments 77 to 81, wherein the NHS functionalized polyolefin glycol is contacted with a reformed secondary amine.

Embodiment 83. The method of any one of Embodiments 77 to 82, wherein the NHS functionalized polyolefin glycol is contacted with a reformed secondary amine from a distilled organic solution.

Embodiment 84. The method of any one of Embodiments 1 to 83, wherein step (c) comprises coupling of the polyolefin-glycol compound and the secondary amine, and step (c) further comprises quenching the coupling by adding an aqueous quench solution comprising potassium carbonate and sodium chloride.

Embodiment 85. The method of Embodiment 84, wherein quenching the coupling comprises:
contacting the aqueous quench solution with a coupling-product mixture formed in step (c) to form a biphasic product mixture comprising i) an organic phase comprising the compound of Formula I and less than 10 wt. % of the secondary amine, and ii) an aqueous phase,
separating the organic phase and the aqueous phase of the biphasic product mixture, and
distilling the organic phase to form a product solution comprising the compound of Formula I and less than 0.12 wt. % of water.

Embodiment 86. The method of Embodiment 85, wherein the organic phase is distilled at a pressure of 0 to 0.2 bar and/or a temperature at or below 70° C. to form the product solution.

Embodiment 87. The method of any one of Embodiments 1 to 86, wherein the method further comprises at least partially purifying the compound of Formula I.

Embodiment 88. The method of Embodiment 87, wherein the compound of Formula I is at least partially purified by silica gel chromatography or polymer resin chromatography.

Embodiment 89. The method of any one of Embodiments 87 to 88, further comprising precipitating the compound of Formula I, the process comprising:
obtaining an ethanol solution of the at least partially purified compound of Formula I, contacting isopropanol with the ethanol solution to form an isopropanol and ethanol mixture,
wherein the compound of Formula I precipitates from the isopropanol and ethanol mixture, and
separating the precipitate of the compound of Formula I from the isopropanol and ethanol mixture.

Embodiment 90. The method of Embodiment 89, wherein isopropanol is contacted with the ethanol solution at an isopropanol:ethanol weight ratio of 3.5:1 to 2.5:1.

Embodiment 91. The method of any one of Embodiments 89 to 90, wherein the precipitate of the compound of Formula I is separated from the isopropanol and ethanol mixture by filtration.

Embodiment 92. The method of any one of Embodiments 1 to 91, wherein the n is 2 and m is 40 to 50.

Embodiment 93. The method of any one of Embodiments 1 to 92, wherein R3 is an alkyl group.

Embodiment 94. The method of any one of Embodiments 1 to 93, wherein R3 is a methyl group.

Embodiment 95. The method of any one of Embodiments 1 to 94, wherein R1 and R2 are independently a linear, saturated, and unsubstituted alkyl group.

Embodiment 96. The method of any one of Embodiments 1 to 95, wherein R1 and R2 independently have a chemical formula selected from the group —(CH2)₇CH3, —(CH2)8CH3, —(CH2)9CH3, —(CH2)10CH3, —(CH2)11CH3, —(CH2)12CH3, —(CH2)13CH3, —(CH2)14CH3, —(CH2)15CH3, —(CH2)16CH3, —(CH2)17CH3, —(CH2)18CH3, and —(CH2)19CH3.

Embodiment 97. The method of any one of Embodiments 1 to 96, wherein R1 is —(CH2)12CH3 and/or R2 is —(CH2)13CH3 group.

Embodiment 98. The method of any one of Embodiments 1 to 97, wherein L has a chemical formula of —(CH₂)^{a′}—X— (CH₂)_{a″}—, wherein a′ and a″ are independently 0, 1, 2, 3, 4, or 5, and X is a linker.

Embodiment 99. The method of Embodiment 98, wherein X is a bond, —HC═CH—, —C≡C—, —C₆H₄—, —O—, or —S—.

Embodiment 100. The method of any one of Embodiments 1 to 99, wherein L is —CH₂—.

Embodiment 101. The method of any one of Embodiments 1 to 100, wherein Formula I is Formula Ia

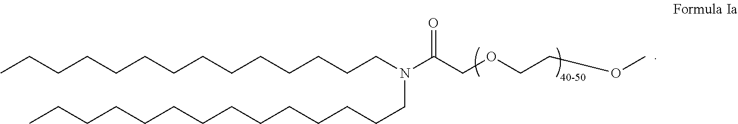

Embodiment 102. A method for forming an amide having a chemical formula of R1-C(O)—NH—R2, wherein R1 and R2 are independently a i) linear or branched or cyclic, ii) saturated or unsaturated, and iii) substituted or unsubstituted hydrocarbon group comprising 8 to 20 carbon atoms, the method comprising
a) contacting a fatty acid having a chemical formula of R1—COOH with 1,1′-Carbonyldiimidazole (CDI) to form a N-acyl imidazole having a chemical formula of R1-C(O)—C3N2H4; and
b) contacting the N-acyl imidazole with a primary amine having a chemical formula of R2—NH₂ to form an amide having a chemical formula of R1—C(O)—NH—R2.

Embodiment 103. The method of Embodiment 102, wherein the fatty acid and CDI have a molar ratio of 1:1.2 to 1.2:1.

Embodiment 104. The method of any one of Embodiments 102 to 103, wherein the N-acyl imidazole is formed at a temperature of 40° C. to 60° C.

Embodiment 105. The method of any one of Embodiments 102 to 104, wherein the fatty acid and CDI are contacted in presence of toluene.

Embodiment 106. The method of any one of Embodiments 102 to 105, wherein the N-acyl imidazole and the primary amine have a molar ratio of 0.9:1 to 1:0.9.

Embodiment 107. The method of any one of Embodiments 102 to 106, wherein the amount of primary amine contacted with the N-acyl imidazole is 0.85 to 1.2 moles of primary amine per mole of the fatty acid used to form the N-acyl imidazole.

Embodiment 108. The method of any one of Embodiments 102 to 107, wherein at least a portion of the primary amine is in a melted form.

Embodiment 109. The method of any one of Embodiments 102 to 108, wherein the amide is formed at a temperature of 40° C. to 60° C.

Embodiment 110. The method of any one of Embodiments 102 to 109, further comprising crystallizing the amide from an amidation-product mixture formed by the reaction of the N-acyl imidazole and primary amine.

Embodiment 111. The method of Embodiment 110, wherein crystallizing the amide comprises adding isopropanol to the amidation-product mixture to form a crystallization mixture and cooling the crystallization mixture.

Embodiment 112. The method of Embodiment 110, wherein crystallizing the amide comprises:
contacting isopropanol with the amidation-product mixture at a temperature greater than 40° C. and at or below 60° C. to form a crystallization mixture;
cooling the crystallization mixture to a temperature of 30° C. to 40° C. to form a slurry containing amide crystals;
maintaining the slurry at a temperature of 30° C. to 40° C., with continuous, periodic, or occasional stirring for at least 1 hour;
cooling the slurry to a temperature of 15° C. to 25° C. with continuous, periodic, or occasional stirring;
maintaining the slurry at a temperature of 15° C. to 25° C. with continuous, periodic, or occasional stirring for at least 0.5 hour; and separating the amide crystals from the slurry.

Embodiment 113. The method of Embodiment 112, wherein the slurry is cooled to a temperature of 15° C. to 25° C. with continuous stirring at 600 rpm or above.

Embodiment 114. The method of any one of Embodiments 112 to 113, wherein the amide crystals are separated from the slurry by filtration.

Embodiment 115. The method of Embodiment 114, further comprising washing the filtered amide crystals with toluene and/or isopropanol, and drying the washed crystals.

Embodiment 116. The method of Embodiment 115, wherein the amide crystals are dried at a temperature of 40° C. to 50° C.

Embodiment 117. The method of any one of Embodiments 110 to 116, wherein the crystallization process is performed in a reactor having a diameter D, the slurry in the reactor has a height H, and H is less than D.

Embodiment 118. The method of Embodiment 117, wherein the reactor comprises an impeller having a diameter DI, and DI:D is 0.35:1 to 0.65:1.

Embodiment 119. The method of any one of Embodiments 110 to 118, wherein the amidation-product mixture comprises less than 4% by weight of the starting primary amine.

Embodiment 120. The method of any one of Embodiments 102 to 119, wherein R1 and R2 are independently a linear, saturated, and unsubstituted alkyl group.

Embodiment 121. The method of any one of Embodiments 102 to 120, wherein R1 is —(CH₂)12CH3, and/or R2 is —(CH2)13CH3 group.

Embodiment 122. A method for forming an amide having a chemical formula of R1—C(O)—NH—R², wherein R1 and R2 are independently a i) linear or branched or cyclic, ii) saturated or unsaturated, and iii) substituted or unsubstituted hydrocarbon group comprising 8 to 20 carbon atoms, the method comprising:
a) contacting a fatty acid having a chemical formula of R1—COOH with an oxychloride to form an acyl chloride having a chemical formula of R1—C(O)—Cl, wherein the oxychloride is selected from the group thionyl chloride, phosphoryl chloride, oxalyl chloride, and any combinations thereof, and
c) contacting the acyl chloride with a primary amine having a chemical formula of R2—NH₂ to form an amide having a chemical formula of R1—C(O)—NH—R2.

Embodiment 123. The method of Embodiment 122, wherein the fatty acid and the oxychloride have a molar ratio of 1:0.8 to 1:2.

Embodiment 124. The method of any one of Embodiments 122 to 123, wherein the fatty acid and the oxychloride are contacted in presence of benzene and dimethylformamide.

Embodiment 125. The method of any one of Embodiments 122 to 124, wherein the fatty acid and the oxychloride are contacted at a temperature of 20° C. to 75° C.

Embodiment 126. The method of any one of Embodiments 122 to 125, wherein the oxychloride is oxayl chloride.

Embodiment 127. The method of any one of Embodiments 122 to 126, wherein the acyl chloride and primary amine are contacted at a temperature of 2° C. to 20° C.

Embodiment 128. The method of any one of Embodiments 122 to 127, wherein the acyl chloride and primary amine are contacted in presence of benzene and triethylamine.

Embodiment 129. The method of any one of Embodiments 122 to 128, wherein the amount of primary amine contacted with the acyl chloride is 0.6 to 1.2 moles of primary amine per mole of the fatty acid used to form the acyl chloride.

Embodiment 130. The method of any one of Embodiments 122 to 129, further comprising crystallizing the amide from an amidation-product mixture formed by the reaction of the acyl chloride and primary amine.

Embodiment 131. The method of Embodiment 130, wherein crystallizing the amide comprises adding isopropanol to the amidation-product mixture to form a crystallization mixture and cooling the crystallization mixture.

Embodiment 132. The method of Embodiment 130, wherein crystallizing the amide comprises:
contacting isopropanol with the amidation-product mixture at a temperature greater than 40° C. and at or below 60° C. to form a crystallization mixture;
cooling the crystallization mixture to a temperature of 30° C. to 40° C. to form a slurry containing amide crystals;
maintaining the slurry at a temperature of 30° C. to 40° C., with continuous, periodic, or occasional stirring for at least 1 hour;
cooling the slurry to a temperature of 15° C. to 25° C. with continuous, periodic, or occasional stirring;
maintaining the slurry at a temperature of 15° C. to 25° C. with continuous, periodic, or occasional stirring for at least 0.5 hour; and separating the amide crystals from the slurry.

Embodiment 133. The method of Embodiment 132, wherein the slurry is cooled to a temperature of 15° C. to 25° C. with continuous stirring at 600 rpm or above.

Embodiment 134. The method of any one of Embodiments 132 to 133, wherein the amide crystals are separated from the slurry by filtration.

Embodiment 135. The method of Embodiment 134, further comprising washing the filtered amide crystals with toluene and/or isopropanol, and drying the washed crystals.

Embodiment 136. The method of Embodiment 135, wherein the amide crystals are dried at a temperature of 40° C. to 50° C.

Embodiment 137. The method of any one of Embodiments 130 to 136, wherein the crystallization process is performed in a reactor having a diameter D, the slurry in the reactor having a height H, and H is less than D.

Embodiment 138. The method of Embodiment 137, wherein the reactor comprises an impeller having a diameter DI, and DI:D is 0.35:1 to 0.65:1.

Embodiment 139. The method of any one of Embodiments 130 to 138, wherein the amidation-product mixture comprises less than 4% by weight of the starting primary amine.

Embodiment 140. The method of any one of Embodiments 122 to 139, wherein R¹ and R² are independently a linear, saturated, and unsubstituted alkyl group.

Embodiment 141. The method of any one of Embodiments 122 to 140, wherein R1 is —(CH2)12CH3, and/or R2 is —(CH2)13CH3 group.

Embodiment 142. A method for producing a compound having a chemical formula of Formula I in solid phase

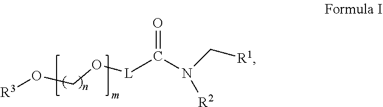

wherein R¹ and R2 are independently a i) linear or branched or cyclic, ii) saturated or unsaturated, and iii) substituted or unsubstituted hydrocarbon group comprising 8 to 20 carbon atoms,

R3 is a hydrocarbon group,

n is an integer from 2 to 5,

m is an integer from 30 to 70, and

L is a linker,

the method comprising:
a) contacting a secondary amine having a chemical formula of R1—CH2—NH—R2 with an polyolefin-glycol compound to couple the secondary amine and the polyolefin-glycol compound to form the compound of Formula I

Embodiment 143. The method of Embodiment 142, wherein the polyolefin-glycol compound is a polyolefin glycol acid having a chemical formula of HOOC—L—(O(CH2)n)m—OR3.

Embodiment 144. The method of Embodiment 143, wherein the secondary amine and the polyolefin glycol acid have a molar ratio of 1:1.2 to 1:1.5.

Embodiment 145. The method of any one of Embodiments 143 to 144, wherein the polyolefin glycol acid is activated by contacting the polyolefin glycol acid with an organic base and a coupling agent to form a coupling solution comprising an activated polyolefin-glycol compound, and the coupling solution is contacted with the secondary amine.

Embodiment 146. The method of Embodiment 145, wherein the organic base is a tertiary amine.

Embodiment 147. The method of Embodiment 146, wherein the tertiary amine is diisopropylethylamine.

Embodiment 148. The method of any one of Embodiments 145 to 147, wherein the coupling agent is 1-propanephosphonic acid cyclic anhydride.

Embodiment 149. The method of any one of Embodiments 145 to 148, wherein the coupling solution is colorless.

Embodiment 150. The method of any one of Embodiments 145 to 149, wherein the coupling solution is formed by contacting the polyolefin glycol acid and the organic base at a molar ratio of 1:3.5 to 1:4.5.

Embodiment 151. The method of any one of Embodiments 145 to 150, wherein the coupling solution is formed by contacting the polyolefin glycol acid and the coupling agent at a molar ratio of 1:1.8 to 1:2.2.

Embodiment 152. The method of any one of Embodiments 145 to 151, wherein the coupling solution is formed by contacting the polyolefin glycol acid with an organic solvent to form a polyolefin glycol solution, distilling the polyolefin glycol solution to form a distilled polyolefin glycol solution, and contacting the distilled polyolefin glycol solution with the base and coupling agent to form the coupling solution.

Embodiment 153. The method of Embodiment 152, wherein the polyolefin glycol solution is distilled at a pressure of 0 to 0.2 bar and/or a temperature at or below 70° C.

Embodiment 154. The method of any one of Embodiments 152 to 153, wherein the distilled polyolefin glycol solution contains less than 0.05 wt. % of water.

Embodiment 155. The method of any one of Embodiments 152 to 154, wherein the polyolefin glycol acid is contacted with toluene to form the polyolefin glycol solution.

Embodiment 156. The method of any one of Embodiments 142 to 155, wherein the compound of Formula I is formed at a temperature of 20° C. to 45° C.

Embodiment 157. The method of Embodiment 142, wherein the polyolefin-glycol compound is a N-hydroxylsuccinimide (NHS) functionalized polyolefin glycol, and has a chemical formula of NHS—O(O)C—L—(O(CH2)n)m—OR3.

Embodiment 158. The method of Embodiment 157, wherein the NHS functionalized polyolefin glycol is contacted with secondary amine at a molar ratio 0.6:1 to 1.2:1.

Embodiment 159. The method of any one of Embodiments 157 to 158, wherein the NHS functionalized polyolefin glycol is contacted with the secondary amine in the presence of a tertiary amine.

Embodiment 160. The method of Embodiment 159, wherein the tertiary amine is triethylamine.

Embodiment 161. The method of any one of Embodiments 157 to 160, wherein the NHS functionalized polyolefin glycol is contacted with the secondary amine at a temperature of 20° C. to 45° C.

Embodiment 162. The method of any one of Embodiments 157 to 161, wherein the NHS functionalized polyolefin glycol is contacted with a reformed secondary amine. Embodiment 163. The method of any one of Embodiments 157 to 162, wherein the NHS functionalized polyolefin glycol is contacted with a reformed secondary amine from a distilled organic solution.

Embodiment 164. The method of any one of Embodiments 142 to 163, wherein the method further comprises quenching the coupling reaction between the polyolefin-glycol compound with the secondary amine by adding an aqueous quench solution comprising potassium carbonate and sodium chloride.

Embodiment 165. The method of Embodiment 164, wherein quenching the coupling comprises:
contacting the aqueous quench solution with a coupling-product mixture to form a biphasic product mixture comprising i) an organic phase comprising the compound of Formula I and less than 10 wt. % of the secondary amine, and ii) an aqueous phase,
separating the organic phase and the aqueous phase of the biphasic product mixture, and
distilling the organic phase to form a product solution comprising the compound of Formula I and less than 0.12 wt. % of water.

Embodiment 166. The method of Embodiment 165, wherein the organic phase is distilled at a pressure of 0 to 0.2 bar and/or a temperature at or below 70° C. to form the product solution.

Embodiment 167. The method of any one of Embodiments 142 to 166, wherein the method further comprises at least partially purifying the compound of Formula I.

Embodiment 168. The method of Embodiment 167, wherein the compound of Formula I is at least partially purified by silica gel chromatography or polymer resin chromatography.

Embodiment 169. The method of any one of Embodiments 167 to 168, further comprising precipitating the compound of Formula I, the process comprising:
obtaining an ethanol solution of the at least partially purified compound of Formula I, contacting isopropanol with the ethanol solution to form an isopropanol and ethanol mixture,
wherein the compound of Formula I precipitates from the isopropanol and ethanol mixture, and
separating the precipitate of the compound of Formula I from the isopropanol and ethanol mixture.

Embodiment 170. The method of Embodiment 169, wherein isopropanol is contacted with the ethanol solution at an isopropanol:ethanol weight ratio of 3.5:1 to 2.5:1.

Embodiment 171. The method of any one of Embodiments 169 to 170, wherein the precipitate of the compound of Formula I is separated from the isopropanol and ethanol mixture by filtration.

Embodiment 172. The method of any one of Embodiments 142 to 171, wherein the n is 2 and m is 40 to 50.

Embodiment 173. The method of any one of Embodiments 142 to 172, wherein R3 is an alkyl group.

Embodiment 174. The method of any one of Embodiments 142 to 173, wherein R3 is a methyl group.

Embodiment 175. The method of any one of Embodiments 142 to 174, wherein R1 and R2 are independently a linear, saturated, and unsubstituted alkyl group.

Embodiment 176. The method of any one of Embodiments 142 to 175, wherein R1 and R2 independently have a chemical formula selected from the group —(CH₂)7CH3, —(CH₂)8CH3, —(CH2)9CH3, —(CH₂)10CH3, —(CH2)11CH3, —(CH2)12CH3, —(CH2)13CH3, —(CH2)14CH3, —(CH2)15CH3, —(CH2)16CH3, —(CH2)17CH3, —(CH2)18CH3, and —(CH2)19CH3.

Embodiment 177. The method of any one of Embodiments 142 to 176, wherein R1 is —(CH2)12CH3 and/or R2 is —(CH2)13CH3 group.

Embodiment 178. The method of any one of Embodiments 142 to 177, wherein L has a chemical formula of —(CH2)a′—X—(CH2)a″—, wherein a′ and a″ are independently 0, 1, 2, 3, 4, or 5, and X is a linker.

Embodiment 179. The method of Embodiment 178, wherein X is selected from a bond, —HC═CH—, —C≡C—, —C6H4—, —O—, or —S—.

Embodiment 180. The method of any one of Embodiments 142 to 179, wherein L is —CH2—.

Embodiment 181. The method of any one of Embodiments 142 to 180, wherein Formula I is Formula Ia

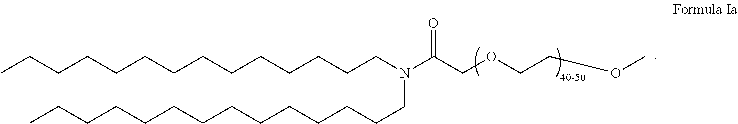

Embodiment 182. A salt comprising a cation having the formula of Formula II

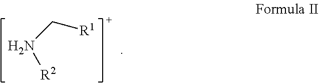

wherein R1 and R2 are independently a i) linear or branched or cyclic, ii) saturated or s unsaturated, and iii) substituted or unsubstituted hydrocarbon group comprising 8 to 20 carbon atoms, and
an anion selected from chloride, bromide, iodide, sulfate, acetate, mesylate, tosylate, (1R)-(-)-10-camphorsulfonate, 1,2-ethanedisulfonate, oxalate, dibenzoyl-L-tartarate, phosphate, L-tartarate, maleate, fumarate, succinate, and malonate.

Embodiment 183. The salt of Embodiment 182, wherein the anion is succinate.

Embodiment 184. The salt of any one of Embodiments 182 to 183, wherein R1 and R2 are independently a linear, saturated, and unsubstituted alkyl group.

Embodiment 185. The salt of any one of Embodiments 182 to 184, wherein R1 is a linear, saturated, and unsubstituted alkyl group containing 13 carbon atoms, and/or R2 is a linear, saturated, and unsubstituted alkyl group containing 14 carbon atoms.

Embodiment 186. The salt of any one of Embodiments 182 to 185, wherein Formula II is Formula IIa

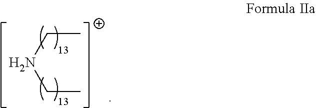

Embodiment 187. The salt of any one of Embodiments 182 to 186, wherein the salt is in a crystallized form.

Embodiment 188. A method for producing the salt in a crystallized form of any one of Embodiments 182 to 187, the method comprising:
a) contacting an amide having a chemical formula of R1—C(O)—NH—R2 with a reducing agent to form a secondary amine having a chemical formula of R1—CH₂—NH—R2; and
b) forming the salt in a crystallized form.

Embodiment 189. The method of Embodiments 188, wherein the reducing agent is a hydride.

Embodiment 190. The method of Embodiment 189, wherein the hydride is lithium aluminum hydride.

Embodiment 191. The method of any one of Embodiments 188 to 190, wherein the amide and the reducing agent have a molar ratio of 1:1 to 1:3.

Embodiment 192. The method of any one of Embodiments 188 to 191, wherein when the amide and reducing agent are contacted, the amide is comprised in an amide solution and the reducing agent is comprised in a reducing agent solution.

Embodiment 193. The method of Embodiment 192, wherein the amide solution further comprises toluene, and/or the reducing agent solution further comprises tetrahydrofuran (THF) and/or 2-methyl THF.

Embodiment 194. The method of any one of Embodiments 192 to 193, wherein the amide solution is formed by contacting crystals of the amide with toluene.

Embodiment 195. The method of any one of Embodiments 188 to 194, wherein the secondary amine is formed at a temperature of 50° C. to 75° C.

Embodiment 196. The method of any one of Embodiments 188 to 195, wherein in step (a) the amide is reduced, and step (a) further comprises quenching the reduction of the amide by adding sodium sulfate.

Embodiment 197. The method of Embodiment 196, wherein quenching the reduction comprises:
contacting a reduction-product mixture comprising the secondary amine formed in step (a) with a slurry comprising sodium sulfate at a temperature of 35° C. to 45° C. to form a quenched reduction-product mixture and residual sodium sulfate;
separating at least a portion of residual sodium sulfate from the quenched reduction-product mixture to form a separated reduction-product mixture comprising the secondary amine.

Embodiment 198. The method of Embodiment 197, wherein the reduction-product mixture comprises less than 4% by weight of the starting amide.

Embodiment 199. The method of any one of Embodiments 196 to 198, wherein 0.5 to 2 moles of sodium sulfate per mole amide is added.

Embodiment 200. The method of any one of Embodiments 197 to 199, wherein the slurry comprising sodium sulfate further comprises THF.

Embodiment 201. The method of any one of Embodiments 197 to 200, wherein the at least a portion of the residual sodium sulfate is separated from the quenched reduction-product mixture by filtration, wherein the separated reduction-product mixture is formed as a filtrate.

Embodiment 202. The method of any one of Embodiments 188 to 201, wherein forming the salt in crystalized form comprises, contacting the secondary amine with an acid to form a salt-forming solution comprising a salt of the secondary amine, and cooling the salt-forming solution to form the salt in crystallized form.

Embodiment 203. The method of any one of Embodiments 188 to 202, wherein forming the salt in crystalized form comprises:
contacting the secondary amine with isopropanol and an acid at a temperature of 50° C. to 60° C. to form a salt-forming solution comprising a salt of the secondary amine;
cooling the salt-forming solution to 30° C. to 45° C. to form salt crystals;
maintaining the salt-forming solution at 30° C. to 45° C. for at least 1 hour;
cooling the salt-forming solution to 15° C. to 25° C.;
separating the salt crystals from the salt-forming solution.

Embodiment 204. The method of any one of Embodiments 202 or 203, wherein the salt crystals are separated from the salt-forming solution by filtering, wherein the salt in crystallized form is obtained as a filtered residue.

Embodiment 205. The method of Embodiment 204, further comprising washing and drying the filtered residue to form a dried, crystallized salt.

Embodiment 206. The method of Embodiment 205, wherein the filtered residue is washed with a toluene and/or isopropanol solution.

Embodiment 207. The method of Embodiment 206, wherein volume % ratio of the toluene and isopropanol in the toluene and/or isopropanol solution is 0.9:1 to 1:0.9.

Embodiment 208. The method of any one of Embodiments 205 to 207, wherein the filtered residue is dried at a pressure of 0 to 0.2 bar and/or a temperature of 40° C. to 50° C.

Embodiment 209. The method of any one of Embodiments 202 to 208, wherein the acid is hydrochloric acid, hydrobromic acid, hydroiodic acid, sulfuric acid, acetic acid, methanesulfonic acid, toluenesulfonic acid, (1R)-(-)-10-camphorsulfonic acid, 1,2-ethanedisulfonic acid, oxalic acid, dibenzoyl-L-tartaric acid, phosphoric acid, L-tartaric acid, maleate, fumaric acid, succinic acid, and/or malonic acid.

Embodiment 210. The method of any one of Embodiments 202 to 209, wherein the acid is succinic acid.

Embodiment 211. A method for producing a compound having a chemical formula of Formula I

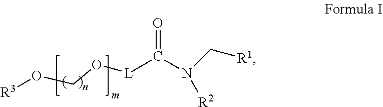

wherein R1 and R2 are independently a i) linear or branched or cyclic, ii) saturated or unsaturated, and iii) substituted or unsubstituted hydrocarbon group comprising 8 to 20 carbon atoms,
R3 is a hydrocarbon group,
n is an integer from 2 to 5,
m is an integer from 30 to 70, and
L is a linker,
the method comprising:
   a) contacting a fatty acid having a chemical formula of R1—COOH, with 1,1′-Carbonyldiimidazole (CDI) to form a N-acyl imidazole having the chemical formula of R1—C(O)—C3N2H4,
   b) contacting the N-acyl imidazole with a primary amine having a chemical formula of R2—NH₂ to form an amide having a chemical formula of R1—C(O)—NH—R2;
   c) contacting the amide with a reducing agent to form a secondary amine having a chemical formula of R1—CH2—NH—R2; and
   d) contacting the secondary amine with a polyolefin-glycol compound to form the compound of Formula I,
wherein the fatty acid and CDI have a molar ratio of 1:1.2 to 1.2:1,
wherein contacting the fatty acid with CDI is performed at a temperature of 40° C. to 60° C.,
wherein the N-acyl imidazole and the primary amine have a molar ratio of 0.9:1 to 1:0.9,
wherein the amount of primary amine contacted with the N-acyl imidazole is 0.85 to 1.2 moles of primary amine per mole of the fatty acid used to form the N-acyl imidazole,
wherein the N-acyl imidazole and the primary amine are contacted at a temperature of 40° C. to 60° C.,
wherein the reducing agent is a hydride, such as lithium aluminum hydride,
wherein the amide and the reducing agent is contacted at a temperature of 50° C. to 75° C.,
wherein the polyolefin-glycol compound is a polyolefin glycol acid having a chemical formula of HOOC—L—(O(CH2)n)m—OR3,
wherein the secondary amine and the polyolefin glycol acid have a molar ratio of 1:1.2 to 1:1.5,
wherein the polyolefin glycol acid is activated by contacting the polyolefin glycol acid with an organic base such as diisopropylethylamine, and a coupling agent such as 1-propanephosphonic acid cyclic anhydride, to form a coupling solution comprising an activated polyolefin-glycol compound, and the coupling solution is contacted with the secondary amine,
wherein the coupling solution is formed by contacting the polyolefin glycol acid and the organic base at a molar ratio of 1:3.5 to 1:4.5,
wherein the coupling solution is formed by contacting the polyolefin glycol acid and the coupling agent at a molar ratio of 1:1.8 to 1:2.2, and
wherein the compound of Formula I is formed at a temperature of 20° C. to 45° C.

Embodiment 212. A method for producing a compound having a chemical formula of Formula I

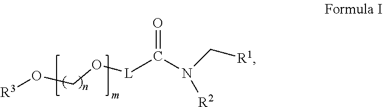

wherein R1 and R2 are independently a i) linear or branched or cyclic, ii) saturated or unsaturated, and iii) substituted or unsubstituted hydrocarbon group comprising 8 to 20 carbon atoms,
R3 is a hydrocarbon group,
n is an integer from 2 to 5,
m is an integer from 30 to 70, and
L is a linker, the method comprising:
   a) contacting a fatty acid having a chemical formula of R1—COOH, with an oxychloride to form an acyl chloride having a chemical formula of R1—C(O)—Cl,
   b) contacting the acyl chloride with a primary amine having a chemical formula of R2—NH2 to form an amide having a chemical formula of R1—C(O)—NH—R2;
   c) contacting the amide with a reducing agent to form a secondary amine having a chemical formula of R1—CH2—NH—R2; and
   d) contacting the secondary amine with a polyolefin-glycol compound to form the compound of Formula I,
wherein the oxychloride is selected from thionyl chloride, phosphoryl chloride, oxalyl chloride, and any combinations thereof,
wherein the fatty acid and the oxychloride have a molar ratio of 1:0.8 to 1:2,
wherein the fatty acid and the oxychloride are contacted at a temperature of 20° C. to 75° C.,
wherein the acyl chloride and primary amine are contacted in the presence of benzene and triethylamine,
wherein the amount of primary amine contacted with the acyl chloride is 0.6 to 1.2 moles of primary amine per mole of the fatty acid used to form the acyl chloride,
wherein the reducing agent is a hydride, such as lithium aluminum hydride,
wherein the amide and the reducing agent is contacted at a temperature of 50° C. to 75° C.,
wherein the polyolefin-glycol compound is a polyolefin glycol acid having a chemical formula of HOOC—L—(O(CH2)n)m—OR3,
wherein the secondary amine and the polyolefin glycol acid have a molar ratio of 1:1.2 to 1:1.5,
wherein the polyolefin glycol acid is activated by contacting the polyolefin glycol acid with an organic base such as diisopropylethylamine, and a coupling agent such as 1-propanephosphonic acid cyclic anhydride, to form a coupling solution comprising an activated polyolefin-glycol compound, and the coupling solution is contacted with the secondary amine,
wherein the coupling solution is formed by contacting the polyolefin glycol acid and the organic base at a molar ratio of 1:3.5 to 1:4.5,
wherein the coupling solution is formed by contacting the polyolefin glycol acid and the coupling agent at a molar ratio of 1:1.8 to 1:2.2, and
wherein the compound of Formula I is formed at a temperature of 20° C. to 45° C.

Embodiment 213. A method for producing a compound having a chemical formula of Formula I

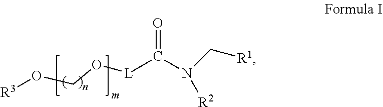

wherein R1 and R2 are independently a i) linear or branched or cyclic, ii) saturated or unsaturated, and iii) substituted or unsubstituted hydrocarbon group comprising 8 to 20 carbon atoms,
R3 is a hydrocarbon group,
n is an integer from 2 to 5,
m is an integer from 30 to 70, and
L is a linker,
the method comprising:
   a) contacting a fatty acid having a chemical formula of R1—COOH, with 1,1′-Carbonyldiimidazole (CDI) to form a N-acyl imidazole having the chemical formula of R1—C(O)—C3N2H4,
   b) contacting the N-acyl imidazole with a primary amine having a chemical formula of R2—NH2 to form an amide having a chemical formula of R1—C(O)—NH—R2;
   c) contacting the amide with a reducing agent to form a secondary amine having a chemical formula of R1—CH₂—NH—R2; and
   d) contacting the secondary amine with a polyolefin-glycol compound to form the compound of Formula I,
wherein the fatty acid and CDI have a molar ratio of 1:1.2 to 1.2:1,
wherein contacting the fatty acid with CDI is performed at a temperature of 40° C. to 60° C.,
wherein the N-acyl imidazole and the primary amine have a molar ratio of 0.9:1 to 1:0.9,
wherein the amount of primary amine contacted with the N-acyl imidazole is 0.85 to 1.2 moles of primary amine per mole of the fatty acid used to form the N-acyl imidazole,
wherein the N-acyl imidazole and the primary amine are contacted at a temperature of 40° C. to 60° C.,
wherein the reducing agent is a hydride, such as lithium aluminum hydride,
wherein the amide and the reducing agent is contacted at a temperature of 50° C. to 75° C.,
wherein the polyolefin-glycol compound is a N-hydroxylsuccinimide (NHS) functionalized polyolefin glycol, and has a chemical formula of NHS—O(O)C—L—(O(CH2)n)m—OR3,
wherein the NHS functionalized polyolefin glycol is contacted with the secondary amine at a molar ratio 0.6:1 to 1.2:1,
wherein the NHS functionalized polyolefin glycol is contacted with the secondary amine in presence of a tertiary amine, such as trimethylamine,
wherein the NHS functionalized polyolefin glycol is contacted with the secondary amine at a temperature of 20° C. to 45° C.

Embodiment 214. A method for producing a compound having a chemical formula of Formula I

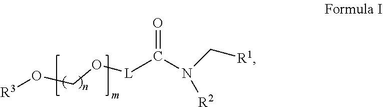

wherein R1 and R2 are independently a i) linear or branched or cyclic, ii) saturated or unsaturated, and iii) substituted or unsubstituted hydrocarbon group comprising 8 to 20 carbon atoms,
R3 is a hydrocarbon group,
n is an integer from 2 to 5,
m is an integer from 30 to 70, and
L is a linker,
the method comprising:
   a) contacting a fatty acid having a chemical formula of R1—COOH, with an oxychloride to form an acyl chloride having a chemical formula of R1—C(O)—Cl,
   b) contacting the acyl chloride with a primary amine having a chemical formula of R2—NH2 to form an amide having a chemical formula of R1—C(O)—NH—R2;
   c) contacting the amide with a reducing agent to form a secondary amine having a chemical formula of R1—CH2—NH—R2; and
   d) contacting the secondary amine with a polyolefin-glycol compound to form the compound of Formula I,
wherein the oxychloride is selected from thionyl chloride, phosphoryl chloride, oxalyl chloride, and any combinations thereof,
wherein the fatty acid and the oxychloride have a molar ratio of 1:0.8 to 1:2,
wherein the fatty acid and the oxychloride are contacted at a temperature of 20° C. to 75° C.,
wherein the acyl chloride and primary amine are contacted in the presence of benzene and triethylamine,
wherein the amount of primary amine contacted with the acyl chloride is 0.6 to 1.2 moles of primary amine per mole of the fatty acid used to form the acyl chloride,
wherein the reducing agent is a hydride, such as lithium aluminum hydride,
wherein the amide and the reducing agent is contacted at a temperature of 50° C. to 75° C.,
wherein the polyolefin-glycol compound is a N-hydroxylsuccinimide (NHS) functionalized polyolefin glycol, and has a chemical formula of NHS—O(O)C—L—(O(CH2)n)m—OR3,
wherein the NHS functionalized polyolefin glycol is contacted with the secondary amine at a molar ratio 0.6:1 to 1.2:1,
wherein the NHS functionalized polyolefin glycol is contacted with the secondary amine in presence of a tertiary amine, such as trimethylamine,
wherein the NHS functionalized polyolefin glycol is contacted with the secondary amine at a temperature of 20° C. to 45° C.

We claim:

1. A method for producing a compound having a chemical formula of Formula I 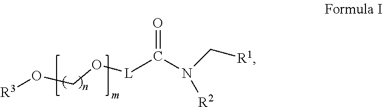
wherein R¹ and R² are independently a i) linear or branched or cyclic, ii) saturated or unsaturated, and iii) substituted or unsubstituted hydrocarbon group comprising 8 to 20 carbon atoms,
R³ is a hydrocarbon group,
n is an integer from 2 to 5,
m is an integer from 30 to 70, and
L is a linker,
the method comprising:
a) forming an amide having a chemical formula of R¹—C(O)—NH—R² from a fatty acid having a chemical formula of R¹—COOH and a primary amine having a chemical formula of R²—NH₂;
b) contacting the amide with a reducing agent to form a secondary amine having a chemical formula of R¹—CH₂—NH—R²; and
c) contacting the secondary amine with a polyolefin-glycol compound to form the compound of Formula I.

2. The method of claim 1, wherein the fatty acid is contacted with 1,1′-Carbonyldiimidazole (CDI) to form a N-acyl imidazole having the chemical formula of of R¹—C(O)—C₃N₂H₄, and the N-acyl imidazole is contacted with the primary amine to form the amide.

3. The method of claim 2, wherein the fatty acid and CDI have a molar ratio of 1:1.2 to 1.2:1.

4. The method of any one of claims 2 to 3, wherein contacting the fatty acid with CDI is performed at a temperature of 40° C. to 60° C.

5. The method of any one of claims 2 to 4, wherein the fatty acid and CDI are contacted in the presence of toluene.

6. The method of any one of claims 1 to 5, wherein the N-acyl imidazole and the primary amine have a molar ratio of 0.9:1 to 1:0.9.

7. The method of any one of claims 2 to 6, wherein the amount of primary amine contacted with the N-acyl imidazole is 0.85 to 1.2 moles of primary amine per mole of the fatty acid used to form the N-acyl imidazole.

8. The method of any one of claims 1 to 7, wherein at least a portion of the primary amine is in a melted form.

9. The method of any one of claims 1 to 8, wherein the N-acyl imidazole and the primary amine are contacted at a temperature of 40° C. to 60° C.

10. The method of claim 1, wherein the fatty acid is contacted with an oxychloride to form an acyl chloride having a chemical formula of R¹—C(O)—Cl, and the acyl chloride is contacted with the primary amine to form the amide, wherein the oxychloride is selected from thionyl chloride, phosphoryl chloride, oxalyl chloride, and any combinations thereof.

11. The method of claim 10, wherein the fatty acid and the oxychloride have a molar ratio of 1:0.8 to 1:2.

12. The method of any one of claims 10 to 11, wherein the fatty acid and the oxychloride are contacted in the presence of benzene and dimethylformamide.

13. The method of any one of claims 10 to 12, wherein the fatty acid and the oxychloride are contacted at a temperature of 20° C. to 75° C.

14. The method of any one of claims 10 to 13, wherein the oxychloride is oxalyl chloride.

15. The method of any one of claims 10 to 14, wherein the acyl chloride and primary amine are contacted at a temperature of 2° C. to 20° C.

16. The method of any one of claims 10 to 15, wherein the acyl chloride and primary amine are contacted in the presence of benzene and triethylamine.

17. The method of any one of claims 10 to 16, wherein the amount of primary amine contacted with the acyl chloride is 0.6 to 1.2 moles of primary amine per mole of the fatty acid used to form the acyl chloride.

18. The method of any one of claims 1 to 17, further comprising crystallizing the amide s from an amidation-product mixture formed in step (a), and using the crystallized amide as at least a portion of the amide in step (b).

19. The method of claim 18, wherein crystallizing the amide comprises adding isopropanol to the amidation-product mixture to form a crystallization mixture and cooling the crystallization mixture.

20. The method of claim 19, wherein crystallizing the amide comprises:
contacting isopropanol with the amidation-product mixture at a temperature greater than 40° C. and at or below 60° C. to form the crystallization mixture;
cooling the crystallization mixture to a temperature of 30° C. to 40° C. to form a slurry containing amide crystals;
maintaining the slurry at a temperature of 30° C. to 40° C., with continuous, periodic, or occasional stirring for at least 1 hour;
cooling the slurry to a temperature of 15° C. to 25° C. with continuous, periodic, or occasional stirring;
maintaining the slurry at a temperature of 15° C. to 25° C. with continuous, periodic, or occasional stirring for at least 0.5 hour; and
separating the amide crystals from the slurry.

21. The method of claim 20, wherein the slurry is cooled to a temperature of 15° C. to 25° C. with continuous stirring at 600 rpm or above.

22. The method of any one of claims 18 to 21, wherein the amide crystals are separated from the slurry by filtration.

23. The method of claim 22, further comprising washing the filtered amide crystals with toluene and/or isopropanol, and drying the washed crystals.

24. The method of claim 23, wherein the amide crystals are dried at a temperature of 40° C. to50° C.

25. The method of any one of claims 18 to 24, wherein the amide crystallization process is performed in a reactor having a diameter D, and the reactor comprises an impeller having a diameter D₁, and D₁:D is 0.35:1 to 0.65:1

26. The method of claim 25, wherein the slurry in the reactor has a height H, and H is less than D.

27. The method of any one of claims 18 to 26, wherein the amidation-product mixture comprises less than 4% by weight of the starting primary amine.

28. The method of any one of claims 1 to 27, wherein the reducing agent is a hydride.

29. The method of claim 28, wherein the hydride is lithium aluminum hydride.

30. The method of any one of claims 1 to 29, wherein the amide and the reducing agent have a molar ratio of 1:1 to 1:3.
